(19) United States
(12) Patent Application Publication
(10) Pub. No.: US 2022/0025454 A1
(43) Pub. Date: Jan. 27, 2022

(54) TUBERCULOSIS RESISTANCE PREDICTION METHOD

(71) Applicant: UNIVERSITÄT ZÜRICH, Zürich (CH)

(72) Inventors: Thorsten BUCH, Schlieren (CH); Prajwal PRAJWAL, Bremgarten (CH); Sebastian DÜMCKE, Berlin (DE)

(73) Assignee: UNIVERSITÄT ZÜRICH, Zürich (CH)

(21) Appl. No.: 17/297,489

(22) PCT Filed: Nov. 29, 2019
(86) PCT No.: PCT/EP2019/083171

§ 371 Date: May 27, 2021

(30) Foreign Application Priority Data
Nov. 29, 2018 (EP) 18209254.4
Feb. 22, 2019 (EP) 19158962.1

(51) Int. Cl. C12Q 1/6869; G16B 30/10; G16B 20/20; C12Q 1/6876

(52) U.S. Cl. CPC C12Q 1/6869; G16B 30/10; C12Q 2600/156; C12Q 1/6876; C12Q 2600/106; G16B 20/20

(57) ABSTRACT

The invention relates to a method for predicting mycobacterial drug resistance by isolating mycobacterial nucleic acid from a sample, obtaining a sample sequence from the nucleic acid, aligning and comparing the sample sequence to a reference sequence and determining for each reference position whether the sample sequence value is the same as a particular sequence value assigned to the position in a table. If both values are the same, a position weight value is assigned to the position. A predictor value is obtained by adding all position weight values and the predictor value is compared to a threshold value. If the predictor value is smaller than the threshold value, drug resistance is predicted. The invention encompasses a system for practicing the method, and the use of certain antibacterial drugs to treat infections by pathogens, resistance of which has been determined by the method of the invention.

The present invention relates to a method for predicting the resistance of a mycobacterial pathogen to common antibacterial drugs.

### BACKGROUND OF THE INVENTION

Tuberculosis (TB) is caused by Mycobacterium tuberculosis. According to the World Health Organisation, about one-quarter of the world's population has latent TB, associated with a 5-15% lifetime risk of falling ill with TB. Patients with compromised immune systems, such as people living with HIV, malnutrition or diabetes, or people who use tobacco, have a much higher risk of falling ill. As a result, TB is one of the top 10 causes of death worldwide. In 2017, 10 million people fell ill with TB, and 1.6 million died from the disease (including 0.3 million among people with HIV).

Anti-TB drugs have been used for decades and strains that are resistant to 1 or more of the most common drugs have been found throughout the world. Drug resistance emerges when drugs are used inappropriately, through incorrect prescription by health care providers, poor quality drugs, and patients stopping treatment prematurely.

Multidrug-resistant tuberculosis (MDR-TB) is a form of TB caused by bacteria that do not respond to isoniazid and rifampicin. MDR-TB is treatable and curable by using second-line drugs. However, second-line treatment options are limited and require extensive chemotherapy with drugs that are expensive and toxic. Extensively drug-resistant TB (XDR-TB) is a more serious form of MDR-TB caused by bacteria that do not respond to the most effective second-line anti-TB drugs, often leaving patients without any further treatment options. MDR-TB remains a public health crisis and a health security threat. WHO estimates that in 2017 there were 558 000 new cases with resistance to rifampicin—the most effective first-line drug—of which 82% had MDR-TB. About 8.5% of MDR-TB cases had extensively drug-resistant TB (XDR-TB).

Worldwide, only 55% of MDR-TB patients are currently successfully treated. In 2016, WHO approved the use of a short, standardised regimen for MDR-TB patients who do not have strains that are resistant to second-line TB medicines. This regimen takes 9-12 months and is much less expensive than the conventional treatment for MDR-TB, which can take up to 2 years. Patients with XDR-TB or resistance to second-line anti-TB drugs cannot use this regimen, however, and need to be put on longer MDR-TB regimens.

The objective of the present invention is to provide improved methods to genetically analyse drug resistance and predict drug resistance or susceptibility in mycobacteria, thereby enabling rapid identification of response profiles in TB patients, and administration of an adequate drug regime to the patient. This objective is attained by the subject matter of the independent claim.

### DESCRIPTION

For the purpose of diagnosing patients with suspected tuberculosis the inventors first determined from a literature search which genes on the genomes of the Mycobacterium tuberculosis complex (MTBC) members have mutations that are predictive for resistance against 17 antibiotics. The inventors employed commercially available primers and probes to amplify the full length genes containing the specific mutations required for the resistance prediction. These specific mutations were predicted through machine learning, validated experimentally and form the present invention.

Several molecular diagnostics approaches based on DNA/RNA analysis (Real Time PCR, Microarray, digital PCR) or protein analysis (Mass Spectrometry) are currently being used in routine diagnostics or are under development. These approaches identify a small number of genes or variants that are unique to the pathogen and are responsible for giving rise to resistance phenotype. They are usually fast and accurate.

Whole genome sequencing of the Mycobacterium genome is currently performed in specialized reference centres with the purpose of identification of new resistance genes/gene variants and to monitor the spread of strains. It usually requires preculture of the bacteria. Whole genome sequence has not yet entered the realm of routine TB diagnostics. Methods have been published for the analysis of whole genome data, including resistance prediction, which consider only a limited number of genes, making them useful only as a supplementary to the culture-based diagnostics. There is currently no molecular approach that can precisely predict susceptibility for all or at least a majority of the relevant antibiotic drugs from the genetic information generated from a molecular assay, therefore molecular diagnostics so far cannot suggest an antibiotic therapy. Whole genome sequencing approaches are largely limited to research activities and not yet translated to routine diagnostics due higher costs than current alternatives as well as lack of CE-IVD certified diagnostic software to interpret the whole genome sequencing data.

Based on the above-mentioned state of the art, the objective of the present invention is to provide means and methods to genetically analyse drug resistance and predict mycobacterial drug susceptibility or mycobacterial drug resistance in a patient.

This objective is attained by the subject-matter of the independent claims of the present specification.

### Terms and Definitions

In the context of the present specification, the term nucleotides refers to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The hybridizing sequence may be composed of any of the above nucleotides, or mixtures thereof.

In the context of the present invention, the term sequencing refers to the determination of the nucleotide sequence of an amplified nucleic acid obtained—for example by polymerase chain reaction (PCR), particularly multiplex PCR—from a mycobacterial nucleic acid sample. Various methods are known in the art, sequencing can be carried out using conventional or next generation sequencing, (e.g. Sanger dideoxy, Illumina, IonTorrent, Nanopore).

In the context of the present invention the term position comparison value is a numerical value that is assigned to a reference position (as specified above) by comparison of a nucleic acid sample sequence to a nucleic acid reference sequence.

One example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.nci.nlm.nih.gov/).

### SUMMARY OF THE INVENTION

The present invention demonstrates a method to diagnose patients with tuberculosis infection and guide optimal therapy by predicting susceptibility or resistance to several antibiotics typically used in treating tuberculosis patients. The method is based on sequencing key regions from the genome of the tuberculosis strain extracted from the patient sample. Contrary to the current standard of care, culturing Tb strains in presence of antibiotics (called phenotypic testing), which takes several weeks, the present method is faster (24-48 hours depending on the protocol) while delivering the same information on drug susceptibility or resistance. Compared with other molecular based assays, such as GenXpert or Hain MTBDR+, the method of the invention includes more key regions on the DNA and can thus provide more and better treatment options. In general, the method makes use of key positions on the DNA of M. tuberculosis complex that were previously not known to be associated with drug resistance or susceptibility. These new positions in turn improve the prediction accuracy of drug resistance or susceptibility. Given a method to interrogate particular positions on the genome of M. tuberculosis (e.g. using whole-genome sequencing or targeted sequencing or PCR+sanger sequencing) the state of the art prediction method will look for any mutation known to be associated with drug resistance or susceptibility from the ReSeqTB database (e.g. from http://erj.ersjournals.com/content/50/6/1701354). If any mutation known to be associated with resistance to a particular drug is found, that isolate is predicted to be phenotypically resistant to that drug. If any mutation conferring susceptibility is found, or the sequence corresponds to wild-type M. tuberculosis, or contains only phylogenetic mutations, then the isolate is predicted to be phenotypically susceptible to that drug (as described in N Engl J Med 2018; 379:1403-1415). The present method improves upon this algorithm, by giving each mutation from a list of relevant mutations a specific weight and then comparing this weighted sum to a pre-determined threshold. This list includes far more positions than known in the art at present, and significantly exceeds those known from the ReSeqTB database. If the sum is above the threshold, the isolate will be predicted to be phenotypically susceptible to the drug, and if the sum is equal or below the threshold, it will be predicted as phenotypically resistant to this drug. The pre-determined threshold and list of mutations included in the sum and the weights are specific for each drug (see Tables A through Q). Together they determine the accuracy of the present method in predicting drug susceptibility or resistance compared to the phenotypic result obtained with the above-mentioned culture method. Furthermore, the current publicly available datasets only contain graded mutations against 13 drugs while the here presented invention also contains mutations and appropriate weights for 4 additional drugs: Ciprofloxacin, para-aminosalycylic acid, cycloserine and rifabutin.

### DETAILED DESCRIPTION OF THE INVENTION

A first aspect of the invention relates to a method for predicting mycobacterial susceptibility or resistance to an antibacterial drug in a patient. This method comprises the following steps:

Mycobacterial nucleic acid is isolated from a sample.

In a sequencing step, a nucleic acid sample sequence is obtained from said mycobacterial nucleic acid.

In a subsequent alignment step, the sample sequence is aligned to a reference sequence, which for the purpose of the present invention is the sequence NC_000962.3 (Mycobacterium tuberculosis H37Rv, complete genome). The reference sequence comprises a plurality of reference positions (RN). In the below Tables, the reference positions (RN) are characterized by their number in the reference sequence; the number of the position is given in the column designated (POS) identified at the top of Tables A, B, C, D, E, F, G, H, I, J, K, L, M, N, O, P and/or Q. Each drug for which a prediction is enabled by the method of the invention is associated to a different table; the drug is identified at the top of the table.

The alignment step yields a sample sequence value S^{R} for each of the reference positions, in other words, the sample sequence value S^{R} is the true sequence value (one of A, T, G, C referring to adenine, thymidine, guanine and cytosine, respectively) of the sample sequence.

The sequencing step is designed to obtain a sample sequence capable of aligning to all of the positions identified in the respective table selected from Tables A, B, C, D, E, F, G, H, I, J, K, L, M, N, O, P and/or Q that is associated to the drug of interest. For embodiments that are aimed at determining drug susceptibility for a plurality, or for all, of the antibacterial drugs of Tables A to Q, the sequencing step needs to be designed to allow aligning to all of the positions in the tables associated with the drugs of interest.

In a subsequent comparison step, the sample sequence obtained in the sequencing step is compared to the reference sequence in at least 90%, particularly ≥95%, of said plurality of reference positions (POS) identified in the one table or several tables, as the case may be, selected from Tables A, B, C, D, E, F, G, H, I, J, K, L, M, N, O, P and/or Q. For each reference position in the table, it is determined whether the sample sequence value S^{R} (the real sequence value at the respective position of the sample sequence) is the same as the sample sequence value S^{N} (the sequence value given under the header S^{N}, to be distinguished from S^{R} and R^{N}) assigned to said position in said table.

If S^{R} is the same as S^{N} (the value given in the predictor table), the value (W) associated to S^{N} in the table is assigned as the position weight value to the position.

If S^{R} is not the same as S^{N}, the value 0 (zero) is assigned as the position weight value to the position.

Thereby, a position weight value assigned to each of said reference positions is obtained. Each of the position weight values is associated and useful only with reference to the antibacterial drug designated in the table from which the position weight values are derived.

In other words, for each of the positions given in a column headed by POS in any of Tables A to Q, which have the value R^{N} in the reference sequence (with R^{N} designated in the entry just right of the position number in the table), the real sample sequence value S^{R} is determined and compared to the sample value S^{N} in the entry to the right of R^{N} in the table. If S^{R} and S^{N} are the same, then the position weight value W given just to the right of S^{N} is noted for the position. If S^{R} and S^{N} are not the same, then the position weight value W for this position is zero.

The denominators R^{N} and S^{N} are written without superscript (RN/SN) in the tables for better legibility.

A predictor value to predict resistance against said antibacterial drug designated in said table, is then obtained by adding all position weight values obtained in the comparison step to obtain the predictor value. In other words, all position weight values obtained from one table are added to give a predictor value associated to the particular drug associated to the table.

Subsequently, the predictor value is compared to the threshold value identified in the header of said table. If the predictor value is equal or smaller than the threshold value, resistance to the antibacterial drug is predicted. Alternatively, if the predictor value is larger than the threshold value, susceptibility to the antibacterial drug is predicted.

### Sampling

The sample had previously been obtained from the patient or an object associated with the patient. While the patient's involvement in the sampling is not implied or necessary, it is to be emphasized that the method is directed at obtaining information about mycobacterial pathogens directly associated to one particular patient in order to predict treatment outcomes for particular drugs in the patient.

In the context of the present specification, the term sample may refer to (inter alia) a swab sample, tissue, body fluids (such as urine, saliva, blood), a cell containing sample, a biopsy sample and clinical patient isolates, any of which are expected to comprise at least one mycobacterial nucleic acid molecule.

Alternatively, the sample may be derived from a clinical setting not associated with a particular patient, but with a plurality of potential patients. Examples may be sample swabs obtained as part of a mycobacterial control effort in clinics, prisons, public transport settings etc.

### Sequencing

Many methods are known to the skilled artisan to arrive at a nucleic acid sequence representative of the mycobacterium expected to be present in the sample. Current approaches include, but are not limited, to massively parallel sequencing methods sometimes summarized under the header of “next generation sequencing” (NGS). The skilled person understands that for the purposes of the present invention, the method of amplification, where appropriate, and the method of sequencing can be selected according to the circumstances, as long as the obtained sequence(s) allow alignment of the sequence that is representative of the sample with the reference sequence.

In certain embodiments, the comparison step comprises comparing said sample sequence to said reference sequence in each of said plurality of reference positions (POS) identified in said table. The skilled person understands that while the greatest possible accuracy of the method is only attained by visiting each and all positions of a given table, omitting a relatively small number of positions, particularly a small number of positions associated with the lowest absolute values of W, will not necessarily impact the accuracy of the method to the point of making it useless, or even making it only as useful as prior art methods for predicting resistance.

In certain embodiments, resistance or susceptibility to an antibacterial drug is determined for more than one drug by performing the method comparing the sample sequence to more than one of Tables A to Q.

In certain embodiments, drug resistance or susceptibility is determined for six antibacterial drugs, particularly for eight antibacterial drugs, more particularly for ten or twelve, or even for fourteen or sixteen antibacterial drugs.

In certain embodiments, drug resistance or susceptibility is determined for a plurality of antibacterial drugs including at least one, particularly two, three or even all of ciprofloxacin, para-aminosalycylic acid, cycloserine and rifabutin.

In certain embodiments, drug resistance is determined for isoniazid, rifampicin, ethambutol, pyrazinamide, streptomycin, ciprofloxacin, moxifloxacin, ofloxacin, amikacin, capreomycin, kanamycin, prothionamide, Ethionamide, para-aminosalicylic acid, cycloserine, rifabutin and levofloxacin.

A second aspect of the invention relates to a system comprising a sequencing means and a computer programmed to carry out the method of any one of the embodiments of the first aspect of the invention. The sequencing means can be an automated sequencer. It is understood that the isolation step is not necessarily performed by the system, but the steps of sequencing, alignment, comparison and summation of the position weight values with subsequent prediction can be performed automatically.

Another aspect of the invention relates to a method of treatment or a use of an antibacterial drug in treatment of a mycobacterial pathogen. The drug is selected from Isoniazid, Rifampicin, Ethambutol, Pyrazinamide, Streptomycin, Ciprofloxacin, Moxifloxacin, Ofloxacin, Amikacin, Capreomycin, Kanamycin, Prothionamide, Ethionamide, Paraaminosalicylic acid, Cycloserine, Rifabutin and Levofloxacin, and it is intended only for use in treatment of a patient suffering from infection by a mycobacterial strain that has been determined to be susceptible to treatment by the particular drug by a method according to any one the above embodiments of the first aspect of the invention.

The invention is further illustrated by the following examples and figures, from which further embodiments and advantages can be drawn. These examples are meant to illustrate the invention but not to limit its scope.

Wherever alternatives for single separable features are laid out herein as “embodiments”, it is to be understood that such alternatives may be combined freely to form discrete embodiments of the invention disclosed herein.

### BRIEF DESCRIPTION OF THE FIGURES

FIG. 1 shows a schematic overview of the method according to the first aspect of the invention.

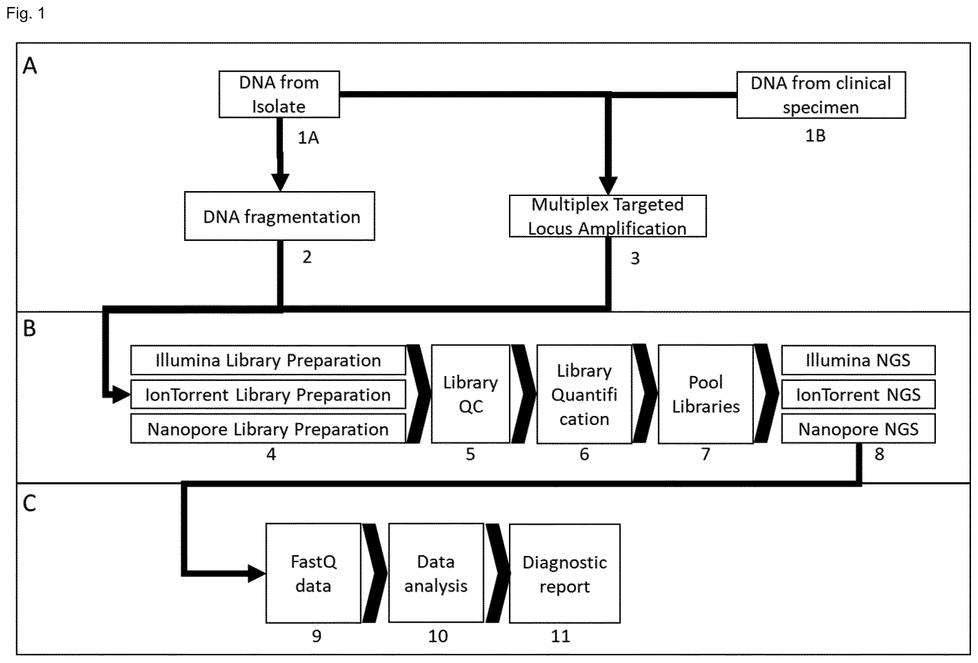

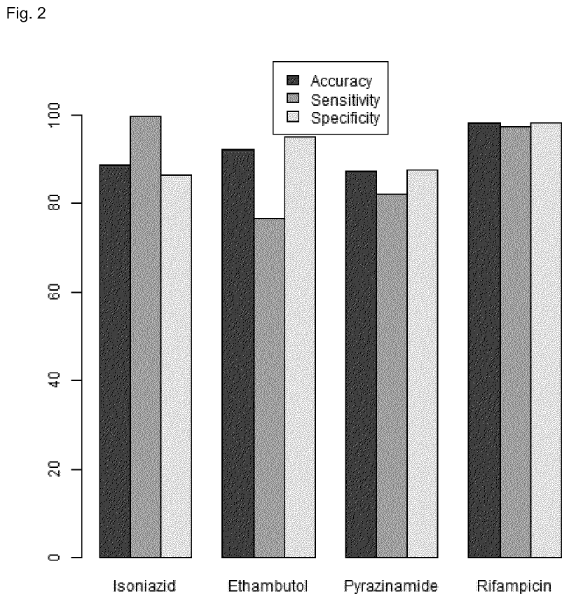

Table 1 shows the threshold values to compare the predictor to for each of 17 antibiotics, in order to determine whether a sample is resistant or susceptible

| Antibacterial drug | Threshold |
|---|---|
| Isoniazid | −1.166 |
| Rifampicin | −0.318 |
| Ethambutol | −1.775 |
| Pyrazinamide | −3.426 |
| Streptomycin | −2.501 |
| Ciprofloxacin | −4.815 |
| Moxifloxacin | −2.547 |
| Ofloxacin | −1.774 |
| Amikacin | −4.729 |
| Capreomycin | −1.531 |
| Kanamycin | −3.033 |
| Prothionamide | −0.551 |
| Ethionamide | −2.589 |
| Para-aminosalicylic acid | −2.598 |
| Cycloserine | −1.859 |
| Rifabutin | −0.605 |
| Levofloxacin | −2.358 |

Tables A through Q show the weights to be added for a set of positions present or absent in the patient sample mycobacterial sequence for each of 17 antibiotics. The result of the addition over all sequences of any one of Tables A to Q is the predictor.

### EXAMPLES

### Example 1

1. DNA is isolated from a clinical specimen or from a cultured isolate (1A) or a clinical specimen (1B) such as sputum, blood, urine, wounds, biopsies, etc. using standard DNA isolation extraction protocols (1) (such as the one described in de Almeida BMC Research Notes 2013 6:561), to yield DNA of good quality,
2. The DNA is fragmented mechanically or enzymatically in the case of whole genome sequencing
3. OR the DNA is amplified in a multiplex PCR reaction with appropriate conditions, for targeted locus amplification, using a PCR primer panel covering within its amplicons mutations found in Tables A-Z,
4. The amplicons are thereafter converted to libraries suitable for sequencing using an NGS platform (either from Illumina, Iontorrent or Oxford nanopore) using manufacturer recommendation of the NGS platform to be used. This does not exclude other and future platforms.
5. The libraries are quality checked using capillary based electrophoresis systems (such as Fragment Analyzer (AATI) or QIAxcel Advanced or other, to determine the size distribution of the libraries.
6. The libraries are quantified using an appropriate fluorometric method (such as Qubit or Quantus or other) or equalized using a library equalizer kit.
7. The libraries are pooled and diluted to appropriate amounts as recommended by the NGS system which they are going to be sequenced on.
8. The libraries are sequenced as per manufacturers' recommendation to achieve adequate read length and depth.
9. The sequencing reads are de-multiplexed and acquired in appropriate formats (e.g. FastQ format) by the software delivered with the NGS instrument or other third-party software.
10. The FastQ sequencing reads are aligned to the reference genome of M. tuberculosis (Genbank accession NC_000962.3) using a short read aligner such as bwa [Li H. (2013) Aligning sequence reads, clone sequences and assembly contigs with BWA-MEM. arXiv:1303.3997v29], SOAP aligner [“SOAP2: an improved ultrafast tool for short read alignment” (2009) BIOINFORMATICS, doi:10.1093/bioinformatics/btp336], stampy [Lunter, G.; Goodson, M. (2011). “Stampy: A statistical algorithm for sensitive and fast mapping of Illumina sequence reads”. Genome Research 21 (6): 936-939. doi:10.1101/gr.111120.1109 or equivalent. The resulting alignment is used to call mutations, e.g. statistical deviations from the reference sequence. This is achieved using software such as SAMtool pileup [Li H, A statistical framework for SNP calling, mutation discovery, association mapping and population genetical parameter estimation from sequencing data, Bioinformatics (2011) 27(21) 2987-939, freebayes [http://arxiv.org/abs/1207.39079] or VarDict [Zhongwu Lai, Aleksandra Markovets, Miika Ahdesmaki, Brad Chapman, Oliver Hofmann, Robert McEwen, Justin Johnson, Brian Dougherty, J. Carl Barrett, Jonathan R. Dry; VarDict: a novel and versatile variant caller for next-generation sequencing in cancer research, Nucleic Acids Research, Volume 44, Issue 11, 20 Jun. 2016, Pages e108, https://doi.org/10.1093/nar/gkw227]. This set of mutations forms the basis for the present invention. For each mutation found in the sample that is also present in any Table A-Z, the weight from the respective table is added together with all other mutations found in the same table. This sum is then compared to the threshold from Table 2 for the respective antibiotic (see Figure). If the sum is smaller or equal to the threshold, the sample is called ‘resistant’ in any other case it is call ‘susceptible’. This procedure is repeated for each antibiotic of interest, yielding an antibiogram.
11. The antibiogram from the previous step along with other potentially interesting information and quality control metrics (e.g. coverage depth and uniformity) is put into a report to be shown, judged and analyzed by the treating physician, which may act on this information to choose an appropriate antibiotic therapy for the patient.

### Example 2

Applying the method of as described in this specification to 5870 sequenced Tb isolates (which can be downloaded from public databases using the following accession list) yields the performance shown in FIG. 2. The sequence data was downloaded from public databases, aligned against the genome of M. tuberculosis (Genbank accession NC_000962.3) and all mutations extracted as described in Step 10 of the example Embodiment, using information from Tables A, B, C and D.

Accuracy, Sensitivity and Specificity were calculated based on the results of phenotypic drug susceptibility testing as published in (The CRyPTIC Consortium et al., N ENGL J MED (2018) 379; 15). These results were obtained with use of an MGIT 960 system (Becton Dickinson), by culture on 7H10 or Löwenstein-Jensen agar or by microscopic-observation drug-susceptibility assay. The published results are considered the phenotypic reference method. Accuracy is the fraction of cases that was predicted correctly (i.e. the phenotypic reference result was predicted). Sensitivity is the proportion of correct predictions from the total of cases that were predicted “R” (i.e. resistant) by the phenotypic reference method. Sensitivity is the fraction of cases that were correctly predicted “S” (i.e. sensitive) from the total number of cases predicted as “S” by the reference method.

Sequence accession numbers for this example:

ERR2512416, ERR2512417, ERR2512418, ERR2512419, ERR2513047, ERR2512421, ERR2512423, ERR2512424, ERR2512425, ERR2512426, ERR2512427, ERR2512390, ERR2512429, ERR2512430, ERR2512431, ERR2512432, ERR2512433, ERR2512434, ERR2512435, ERR2512436, ERR2512437, ERR2512438, ERR2512439, ERR2512440, ERR2512441, ERR2512442, ERR2512443, ERR2512444, ERR2512445, ERR2512446, ERR2512447, ERR2512448, ERR2512449, ERR2512450, ERR2512451, ERR2512452, ERR2512453, ERR2512454, ERR2512455, ERR2512456, ERR2512457, ERR2512458, ERR2512616, ERR2512461, ERR2512462, ERR2512463, ERR2512464, ERR2512465, ERR2512466, ERR2512467, ERR2512469, ERR2512470, ERR2512471, ERR2512472, ERR2512473, ERR2512474, ERR2512475, ERR2512476, ERR2512477, ERR2512478, ERR2512479, ERR2513636, ERR2512481, ERR2512482, ERR2512483, ERR2512484, ERR2512485, ERR2512486, ERR2512487, ERR2512404, ERR2512489, ERR2512490, ERR2512491, ERR2512492, ERR2512493, ERR2512494, ERR2512495, ERR2512496, ERR2512497, ERR2512498, ERR2512499, ERR2512501, ERR2512502, ERR2512503, ERR2512504, ERR2512505, ERR2512506, ERR2512507, ERR2512508, ERR2512509, ERR2512510, ERR2512511, ERR2512512, ERR2512513, ERR2512514, ERR2512515, ERR2512517, ERR2512518, ERR2512519, ERR2512520, ERR2512521, ERR2512522, ERR2512523, ERR2512524, ERR2512526, ERR2512527, ERR2512528, ERR2512529, ERR2512634, ERR2512531, ERR2512532, ERR2512534, ERR2512535, ERR2512536, ERR2512537, ERR2512538, ERR2512539, ERR2512389, ERR2512541, ERR2512542, ERR2512543, ERR2512544, ERR2512545, ERR2512546, ERR2512398, ERR2512548, ERR2512549, ERR2512550, ERR2512551, ERR2512552, ERR2512553, ERR2512554, ERR2512555, ERR2512556, ERR2512557, ERR2512558, ERR2512559, ERR2513411, ERR2512561, ERR2512562, ERR2512563, ERR2512564, ERR2512565, ERR2512566, ERR2512567, ERR2512568, ERR2512569, ERR2512570, ERR2512571, ERR2512572, ERR2512573, ERR2512574, ERR2512575, ERR2512576, ERR2512577, ERR2512578, ERR2512579, ERR2512580, ERR2512581, ERR2512582, ERR2512583, ERR2512584, ERR2512585, ERR2512586, ERR2512587, ERR2512588, ERR2512590, ERR2512591, ERR2512592, ERR2512593, ERR2512594, ERR2512595, ERR2513907, ERR2512598, ERR2512599, ERR2512600, ERR2512602, ERR2512603, ERR2512604, ERR2512605, ERR2512606, ERR2512607, ERR2512608, ERR2512609, ERR2512610, ERR2512611, ERR2512612, ERR2512613, ERR2512614, ERR2512615, ERR2513464, ERR2512617, ERR2512618, ERR2512619, ERR2513624, ERR2512621, ERR2512622, ERR2512624, ERR2512625, ERR2512627, ERR2512628, ERR2512630, ERR2512631, ERR2512632, ERR2512633, ERR2512560, ERR2512635, ERR2512636, ERR2512637, ERR2512638, ERR2512639, ERR2512640, ERR2512643, ERR2512644, ERR2512645, ERR2512646, ERR2512647, ERR2512648, ERR2512649, ERR2512650, ERR2512651, ERR2512652, ERR2512653, ERR2512654, ERR2512655, ERR2512656, ERR2512657, ERR2512658, ERR2512659, ERR2512660, ERR2512661, ERR2512662, ERR2513783, ERR2512665, ERR2512666, ERR2512667, ERR2512668, ERR2512669, ERR2512670, ERR2512671, ERR2512672, ERR2513305, ERR2512674, ERR2512675, ERR2512676, ERR2512677, ERR2512678, ERR2512679, ERR2512680, ERR2512681, ERR2512682, ERR2512683, ERR2512685, ERR2512686, ERR2512687, ERR2512688, ERR2512689, ERR2512690, ERR2513583, ERR2512692, ERR2512693, ERR2512694, ERR2512695, ERR2512696, ERR2512391, ERR2512698, ERR2512699, ERR2512700, ERR2512397, ERR2512702, ERR2512765, ERR2512704, ERR2512705, ERR2512706, ERR2512707, ERR2512708, ERR2512709, ERR2512710, ERR2512711, ERR2512712, ERR2512713, ERR2512714, ERR2512715, ERR2512814, ERR2512718, ERR2512719, ERR2512720, ERR2512547, ERR2512722, ERR2512723, ERR2512724, ERR2512726, ERR2512727, ERR2512728, ERR2512729, ERR2512730, ERR2512731, ERR2512732, ERR2512733, ERR2512734, ERR2512735, ERR2512736, ERR2512737, ERR2512738, ERR2512739, ERR2512740, ERR2512741, ERR2512742, ERR2512743, ERR2512744, ERR2512745, ERR2512746, ERR2512747, ERR2512748, ERR2512749, ERR2512751, ERR2512752, ERR2512753, ERR2512754, ERR2512755, ERR2512756, ERR2512757, ERR2512758, ERR2512759, ERR2512760, ERR2512761, ERR2512762, ERR2512763, ERR2512764, ERR2512406, ERR2512766, ERR2512768, ERR2512770, ERR2512771, ERR2512772, ERR2512773, ERR2512774, ERR2512775, ERR2512776, ERR2512777, ERR2512778, ERR2512779, ERR2512780, ERR2512782, ERR2512783, ERR2512784, ERR2512785, ERR2512786, ERR2512787, ERR2512788, ERR2512789, ERR2512790, ERR2512791, ERR2512792, ERR2512793, ERR2512794, ERR2512795, ERR2512796, ERR2512798, ERR2512799, ERR2512800, ERR2512801, ERR2512802, ERR2512803, ERR2512804, ERR2512805, ERR2512806, ERR2512807, ERR2512809, ERR2512810, ERR2512811, ERR2512812, ERR2512813, ERR2513337, ERR2512815, ERR2512816, ERR2512817, ERR2512818, ERR2512819, ERR2512820, ERR2512821, ERR2512822, ERR2512823, ERR2512824, ERR2512825, ERR2512827, ERR2512828, ERR2512829, ERR2512830, ERR2512831, ERR2512832, ERR2512833, ERR2512834, ERR2512835, ERR2512836, ERR2512837, ERR2512838, ERR2512839, ERR2512840, ERR2512842, ERR2512843, ERR2512844, ERR2512845, ERR2512846, ERR2512847, ERR2512848, ERR2512849, ERR2512850, ERR2512852, ERR2512853, ERR2512854, ERR2512855, ERR2512856, ERR2512857, ERR2512858, ERR2512859, ERR2512860, ERR2512861, ERR2512862, ERR2512864, ERR2512865, ERR2512866, ERR2512867, ERR2512868, ERR2512673, ERR2512870, ERR2512871, ERR2512872, ERR2512873, ERR2512874, ERR2512875, ERR2512877, ERR2512396, ERR2512879, ERR2512880, ERR2512881, ERR2512882, ERR2512884, ERR2512885, ERR2512886, ERR2512887, ERR2513712, ERR2512889, ERR2512890, ERR2512891, ERR2512892, ERR2512893, ERR2512894, ERR2512895, ERR2512896, ERR2512897, ERR2512898, ERR2512899, ERR2512900, ERR2512901, ERR2512902, ERR2512903, ERR2512904, ERR2512905, ERR2512906, ERR2512907, ERR2512908, ERR2512909, ERR2512910, ERR2512911, ERR2512912, ERR2512913, ERR2512914, ERR2512915, ERR2512916, ERR2512917, ERR2512918, ERR2512919, ERR2512920, ERR2512921, ERR2512922, ERR2512924, ERR2512925, ERR2512927, ERR2512928, ERR2512929, ERR2512930, ERR2512931, ERR2512932, ERR2512933, ERR2512934, ERR2512935, ERR2512936, ERR2512937, ERR2512938, ERR2512939, ERR2512940, ERR2512942, ERR2512943, ERR2512944, ERR2512945, ERR2512946, ERR2512947, ERR2512948, ERR2512949, ERR2512412, ERR2512951, ERR2512952, ERR2512953, ERR2512954, ERR2512955, ERR2512956, ERR2512957, ERR2512958, ERR2512959, ERR2512961, ERR2512962, ERR2512963, ERR2512964, ERR2512965, ERR2512966, ERR2512967, ERR2512968, ERR2512969, ERR2512970, ERR2512971, ERR2512974, ERR2512975, ERR2512976, ERR2512977, ERR2512978, ERR2512979, ERR2512980, ERR2512981, ERR2512982, ERR2512983, ERR2512985, ERR2512986, ERR2512987, ERR2512988, ERR2512989, ERR2512990, ERR2512991, ERR2512992, ERR2512993, ERR2512994, ERR2512996, ERR2512997, ERR2512998, ERR2512999, ERR2513000, ERR2513001, ERR2513002, ERR2513003, ERR2513004, ERR2513005, ERR2513006, ERR2513007, ERR2513008, ERR2513010, ERR2513012, ERR2513013, ERR2513014, ERR2513015, ERR2513016, ERR2513514, ERR2513018, ERR2513403, ERR2513020, ERR2512402, ERR2513022, ERR2513023, ERR2513024, ERR2513025, ERR2513027, ERR2513028, ERR2513029, ERR2513030, ERR2513031, ERR2513032, ERR2513226, ERR2513034, ERR2513036, ERR2513037, ERR2513038, ERR2513039, ERR2513040, ERR2513041, ERR2513042, ERR2513044, ERR2513045, ERR2513046, ERR2513731, ERR2513048, ERR2513049, ERR2513050, ERR2513051, ERR2513052, ERR2513053, ERR2513054, ERR2513055, ERR2513056, ERR2513057, ERR2513058, ERR2513059, ERR2513060, ERR2513061, ERR2513062, ERR2513063, ERR2513064, ERR2513065, ERR2513066, ERR2513068, ERR2513069, ERR2513070, ERR2513072, ERR2513035, ERR2513074, ERR2513075, ERR2513076, ERR2513077, ERR2513078, ERR2513079, ERR2513080, ERR2513081, ERR2513082, ERR2513083, ERR2513084, ERR2513085, ERR2513086, ERR2513087, ERR2513088, ERR2513089, ERR2513090, ERR2513091, ERR2513092, ERR2513093, ERR2513094, ERR2513095, ERR2513096, ERR2513097, ERR2513098, ERR2513099, ERR2513100, ERR2513101, ERR2513102, ERR2513103, ERR2513105, ERR2513106, ERR2513107, ERR2513108, ERR2513109, ERR2513110, ERR2513111, ERR2513112, ERR2513113, ERR2513114, ERR2513115, ERR2513116, ERR2513117, ERR2513118, ERR2513119, ERR2513120, ERR2513121, ERR2513122, ERR2513123, ERR2513124, ERR2513125, ERR2513126, ERR2513127, ERR2513128, ERR2513129, ERR2513130, ERR2513131, ERR2513132, ERR2513133, ERR2513134, ERR2513135, ERR2513136, ERR2513137, ERR2513138, ERR2513139, ERR2513140, ERR2513141, ERR2513142, ERR2513143, ERR2513144, ERR2513145, ERR2513146, ERR2513147, ERR2513148, ERR2513149, ERR2513150, ERR2513151, ERR2513152, ERR2513153, ERR2513154, ERR2513155, ERR2513019, ERR2513157, ERR2513158, ERR2513159, ERR2513161, ERR2513162, ERR2513164, ERR2513165, ERR2513166, ERR2513167, ERR2513168, ERR2513169, ERR2513170, ERR2513171, ERR2513172, ERR2513173, ERR2513174, ERR2513175, ERR2513176, ERR2513177, ERR2513178, ERR2513180, ERR2513182, ERR2513183, ERR2513184, ERR2513185, ERR2513186, ERR2513187, ERR2513188, ERR2513189, ERR2513190, ERR2513191, ERR2513192, ERR2513193, ERR2513194, ERR2513195, ERR2513196, ERR2513197, ERR2513198, ERR2513199, ERR2513200, ERR2513201, ERR2513202, ERR2513203, ERR2513204, ERR2513205, ERR2513206, ERR2513207, ERR2513208, ERR2512480, ERR2513210, ERR2513211, ERR2513212, ERR2513213, ERR2513214, ERR2512878, ERR2513216, ERR2513217, ERR2513218, ERR2513219, ERR2513220, ERR2513221, ERR2513222, ERR2513223, ERR2513224, ERR2513225, ERR2513227, ERR2513228, ERR2512409, ERR2513231, ERR2513232, ERR2513233, ERR2513234, ERR2513235, ERR2513236, ERR2513237, ERR2513238, ERR2513239, ERR2513240, ERR2513241, ERR2513242, ERR2513243, ERR2513244, ERR2513245, ERR2513246, ERR2513247, ERR2513248, ERR2513975, ERR2513250, ERR2513251, ERR2513252, ERR2513253, ERR2513254, ERR2513255, ERR2513256, ERR2513257, ERR2513258, ERR2513259, ERR2513260, ERR2513261, ERR2513263, ERR2513264, ERR2513265, ERR2513266, ERR2513267, ERR2513268, ERR2513269, ERR2513270, ERR2513272, ERR2513273, ERR2513274, ERR2513275, ERR2513276, ERR2513277, ERR2513278, ERR2513279, ERR2513280, ERR2513281, ERR2513282, ERR2513283, ERR2513284, ERR2513285, ERR2513286, ERR2513287, ERR2513288, ERR2513289, ERR2513290, ERR2513291, ERR2513292, ERR2513293, ERR2513294, ERR2513296, ERR2513297, ERR2513298, ERR2513299, ERR2513300, ERR2513301, ERR2513302, ERR2513303, ERR2513304, ERR2513638, ERR2513306, ERR2513307, ERR2513308, ERR2513309, ERR2513310, ERR2513311, ERR2513312, ERR2513313, ERR2512410, ERR2513315, ERR2513316, ERR2513317, ERR2513318, ERR2513319, ERR2513320, ERR2513321, ERR2513322, ERR2513323, ERR2513324, ERR2513325, ERR2513326, ERR2513327, ERR2513328, ERR2513329, ERR2513330, ERR2513331, ERR2513532, ERR2513334, ERR2513335, ERR2513336, ERR2512717, ERR2513338, ERR2513339, ERR2513340, ERR2513341, ERR2513342, ERR2513343, ERR2513344, ERR2513345, ERR2513346, ERR2513347, ERR2513348, ERR2513349, ERR2513350, ERR2513351, ERR2513352, ERR2513353, ERR2513354, ERR2513355, ERR2513356, ERR2513357, ERR2513358, ERR2513359, ERR2513360, ERR2513361, ERR2513362, ERR2513363, ERR2513364, ERR2513365, ERR2513366, ERR2512540, ERR2513368, ERR2513369, ERR2513370, ERR2513371, ERR2512620, ERR2513373, ERR2513374, ERR2513375, ERR2513376, ERR2513377, ERR2513378, ERR2513380, ERR2513381, ERR2513383, ERR2513384, ERR2513385, ERR2513386, ERR2513832, ERR2513388, ERR2513389, ERR2513390, ERR2513391, ERR2513392, ERR2513393, ERR2513394, ERR2513395, ERR2513397, ERR2513398, ERR2513399, ERR2513230, ERR2513401, ERR2513402, ERR2513156, ERR2513404, ERR2513405, ERR2513406, ERR2513407, ERR2513408, ERR2512405, ERR2513410, ERR2513412, ERR2513413, ERR2513414, ERR2513415, ERR2513416, ERR2513417, ERR2513418, ERR2513419, ERR2513422, ERR2513423, ERR2513424, ERR2513425, ERR2513426, ERR2513427, ERR2513428, ERR2513429, ERR2513430, ERR2513431, ERR2513432, ERR2513433, ERR2513434, ERR2513435, ERR2513437, ERR2513438, ERR2513439, ERR2513440, ERR2513441, ERR2513442, ERR2513443, ERR2513444, ERR2513445, ERR2513446, ERR2513447, ERR2513448, ERR2513449, ERR2513450, ERR2513451, ERR2513452, ERR2513453, ERR2513454, ERR2513455, ERR2513456, ERR2513457, ERR2513458, ERR2513459, ERR2513460, ERR2513461, ERR2513462, ERR2513463, ERR2513387, ERR2513465, ERR2513466, ERR2513467, ERR2513468, ERR2513469, ERR2513470, ERR2513471, ERR2513472, ERR2513473, ERR2513474, ERR2513475, ERR2513476, ERR2513477, ERR2513478, ERR2513479, ERR2513480, ERR2513481, ERR2513482, ERR2513483, ERR2513484, ERR2513485, ERR2513486, ERR2513487, ERR2513488, ERR2513489, ERR2513490, ERR2513491, ERR2513492, ERR2513493, ERR2513494, ERR2513495, ERR2513496, ERR2513497, ERR2513498, ERR2513499, ERR2513500, ERR2513501, ERR2513502, ERR2513503, ERR2513504, ERR2513505, ERR2513506, ERR2513507, ERR2513508, ERR2513510, ERR2513512, ERR2513513, ERR2513333, ERR2513515, ERR2513516, ERR2513518, ERR2513519, ERR2513520, ERR2513521, ERR2513523, ERR2513524, ERR2513525, ERR2513526, ERR2513527, ERR2513528, ERR2513529, ERR2513530, ERR2513531, ERR2512414, ERR2513533, ERR2513534, ERR2513535, ERR2513536, ERR2513537, ERR2513538, ERR2513539, ERR2513540, ERR2513541, ERR2513542, ERR2513543, ERR2513544, ERR2513545, ERR2513546, ERR2513547, ERR2513548, ERR2513549, ERR2513550, ERR2513551, ERR2513552, ERR2513553, ERR2513554, ERR2513555, ERR2513556, ERR2513557, ERR2513558, ERR2513559, ERR2513560, ERR2513561, ERR2513033, ERR2513563, ERR2513886, ERR2513566, ERR2513567, ERR2513568, ERR2513569, ERR2513570, ERR2513571, ERR2513573, ERR2513574, ERR2513575, ERR2513576, ERR2513577, ERR2513578, ERR2513579, ERR2513580, ERR2513581, ERR2513582, ERR2512413, ERR2513584, ERR2513585, ERR2513586, ERR2513587, ERR2513588, ERR2513589, ERR2513590, ERR2513591, ERR2513592, ERR2513593, ERR2513594, ERR2513595, ERR2513596, ERR2513597, ERR2513598, ERR2513599, ERR2513600, ERR2513601, ERR2513602, ERR2513603, ERR2513604, ERR2513606, ERR2513608, ERR2513609, ERR2513610, ERR2513611, ERR2513612, ERR2513613, ERR2513614, ERR2513615, ERR2513616, ERR2513617, ERR2513618, ERR2513619, ERR2513620, ERR2513621, ERR2513622, ERR2513623, ERR2512401, ERR2513626, ERR2513627, ERR2513628, ERR2513629, ERR2513630, ERR2513632, ERR2513633, ERR2513634, ERR2513637, ERR2512385, ERR2513639, ERR2513640, ERR2513642, ERR2513382, ERR2513644, ERR2513645, ERR2513646, ERR2513647, ERR2513648, ERR2513649, ERR2513650, ERR2513651, ERR2513652, ERR2513653, ERR2513654, ERR2513656, ERR2513659, ERR2513660, ERR2513661, ERR2513664, ERR2513665, ERR2513666, ERR2513667, ERR2513668, ERR2513669, ERR2513670, ERR2513673, ERR2513674, ERR2513675, ERR2513676, ERR2513677, ERR2513678, ERR2513679, ERR2513680, ERR2513681, ERR2513682, ERR2513683, ERR2513684, ERR2513685, ERR2513686, ERR2513687, ERR2513688, ERR2513689, ERR2513690, ERR2513691, ERR2513692, ERR2513693, ERR2513694, ERR2513695, ERR2513696, ERR2513697, ERR2513698, ERR2513699, ERR2513700, ERR2513701, ERR2513702, ERR2513703, ERR2513704, ERR2513705, ERR2513706, ERR2513215, ERR2513708, ERR2513709, ERR2513710, ERR2513711, ERR2512411, ERR2513713, ERR2513714, ERR2513715, ERR2513716, ERR2513717, ERR2513718, ERR2513719, ERR2513720, ERR2513721, ERR2513722, ERR2513723, ERR2513724, ERR2513725, ERR2513726, ERR2513727, ERR2513728, ERR2513729, ERR2513730, ERR2512392, ERR2513732, ERR2513733, ERR2513734, ERR2513736, ERR2513737, ERR2513738, ERR2513739, ERR2513740, ERR2513741, ERR2513743, ERR2513744, ERR2513745, ERR2513746, ERR2513747, ERR2513748, ERR2513749, ERR2513751, ERR2513752, ERR2513753, ERR2513754, ERR2513755, ERR2513756, ERR2513757, ERR2513758, ERR2513759, ERR2513760, ERR2513761, ERR2513762, ERR2513763, ERR2513764, ERR2513765, ERR2513766, ERR2513767, ERR2513768, ERR2513769, ERR2513771, ERR2513772, ERR2513773, ERR2513774, ERR2513775, ERR2513777, ERR2513778, ERR2513779, ERR2513780, ERR2513781, ERR2513782, ERR2512394, ERR2513784, ERR2513785, ERR2513786, ERR2513787, ERR2513788, ERR2513789, ERR2513790, ERR2513792, ERR2513793, ERR2513795, ERR2513796, ERR2513797, ERR2513798, ERR2513799, ERR2513800, ERR2513801, ERR2513802, ERR2513803, ERR2513804, ERR2513805, ERR2513806, ERR2513807, ERR2513808, ERR2513809, ERR2513810, ERR2513812, ERR2513813, ERR2513814, ERR2513815, ERR2513816, ERR2513817, ERR2513818, ERR2513819, ERR2513821, ERR2513822, ERR2513823, ERR2513824, ERR2513825, ERR2513827, ERR2513828, ERR2513635, ERR2513830, ERR2513831, ERR2512863, ERR2513833, ERR2513834, ERR2512950, ERR2513836, ERR2513837, ERR2513838, ERR2513839, ERR2513840, ERR2513841, ERR2513842, ERR2513843, ERR2513844, ERR2513845, ERR2513846, ERR2513847, ERR2513848, ERR2513849, ERR2513851, ERR2513852, ERR2513409, ERR2513854, ERR2513855, ERR2513856, ERR2513857, ERR2513858, ERR2513859, ERR2513860, ERR2513861, ERR2513862, ERR2513863, ERR2513864, ERR2513865, ERR2513866, ERR2513867, ERR2513868, ERR2513869, ERR2513870, ERR2513871, ERR2513872, ERR2513873, ERR2513874, ERR2513875, ERR2513876, ERR2513877, ERR2513878, ERR2513879, ERR2513880, ERR2513881, ERR2513882, ERR2513883, ERR2513884, ERR2513885, ERR2512407, ERR2513887, ERR2513888, ERR2513889, ERR2513890, ERR2513891, ERR2513892, ERR2513893, ERR2513894, ERR2513895, ERR2513896, ERR2513897, ERR2513898, ERR2513899, ERR2513900, ERR2513901, ERR2513902, ERR2513903, ERR2513904, ERR2513905, ERR2513906, ERR2512395, ERR2513908, ERR2513909, ERR2513910, ERR2513911, ERR2513912, ERR2513913, ERR2513914, ERR2513915, ERR2513916, ERR2513917, ERR2513918, ERR2513919, ERR2513920, ERR2513921, ERR2513922, ERR2513923, ERR2513924, ERR2513925, ERR2513926, ERR2513927, ERR2513928, ERR2513929, ERR2513930, ERR2513931, ERR2513932, ERR2513933, ERR2513934, ERR2513935, ERR2513936, ERR2513937, ERR2513938, ERR2513939, ERR2513940, ERR2513941, ERR2513942, ERR2513943, ERR2513944, ERR2513945, ERR2513946, ERR2513947, ERR2513948, ERR2513949, ERR2513950, ERR2513951, ERR2513952, ERR2513953, ERR2513954, ERR2513955, ERR2513956, ERR2513958, ERR2513960, ERR2513961, ERR2513962, ERR2513963, ERR2513965, ERR2513966, ERR2513967, ERR2513968, ERR2513969, ERR2513970, ERR2513971, ERR2513972, ERR2513973, ERR2513974, ERR2513976, ERR2513978, ERR2513979, ERR2513980, ERR2512597, ERR2513981, ERR2512399, ERR2513982, ERR2513983, ERR2513984, ERR2513985, ERR2513986, ERR2513987, ERR2513988, ERR2513989, ERR2513990, ERR2513991, ERR2513992, ERR2513993, ERR2513994, ERR2513995, ERR2513996, ERR2513997, ERR2513998, ERR2513999, ERR2514000, ERR2514001, ERR2514002, ERR2514003, ERR2514004, ERR2514005, ERR2514007, ERR2514008, ERR2514009, ERR2514010, ERR2514011, ERR2514012, ERR2514013, ERR2512386, ERR2514014, ERR2514015, ERR2514016, ERR2514017, ERR2514018, ERR2514019, ERR2514020, ERR2514021, ERR2514023, ERR2514024, ERR2514025, ERR2514026, ERR2514027, ERR2514028, ERR2514029, ERR2514030, ERR2514031, ERR2514032, ERR2514033, ERR2514034, ERR2514035, ERR2514036, ERR2514037, ERR2512888, ERR2514038, ERR2514039, ERR2514041, ERR2514042, ERR2513249, ERR2512388, ERR2514043, ERR2514044, ERR2514045, ERR2514046, ERR2514047, ERR2514048, ERR2514049, ERR2514050, ERR2514051, ERR2514052, ERR2514053, ERR2514054, ERR2514055, ERR2512973, ERR2514056, ERR2514057, ERR2514058, ERR2514059, ERR2514060, ERR2514061, ERR2514062, ERR2512530, ERR2514064, ERR2514065, ERR2514066, ERR2514067, ERR2514068, ERR2514069, ERR2514070, ERR2514071, ERR2514072, ERR2514073, ERR2514074, ERR2514075, ERR2514076, ERR2514077, ERR2514078, ERR2514079, ERR2514080, ERR2514081, ERR2514082, ERR2514083, ERR2514084, ERR2514085, ERR2514086, ERR2514087, ERR2514088, ERR2514089, ERR2514090, ERR2514091, ERR2514093, ERR2514094, ERR2514095, ERR2514096, ERR2513564, ERR2514097, ERR2514098, ERR2514099, ERR2514100, ERR2514101, ERR2514102, ERR2514103, ERR2514104, ERR2514105, ERR2514106, ERR2514107, ERR2514108, ERR2514109, ERR2514110, ERR2514111, ERR2514112, ERR2514113, ERR2514114, ERR2514115, ERR2514116, ERR2514117, ERR2514118, ERR2514119, ERR2514120, ERR2513372, ERR2514121, ERR2514122, ERR2514123, ERR2514124, ERR2514125, ERR2514126, ERR2514127, ERR2514128, ERR2514129, ERR2514130, ERR2514131, ERR2514132, ERR2514133, ERR2514134, ERR2514135, ERR2514136, ERR2513209, ERR2514138, ERR2514139, ERR2514140, ERR2514141, ERR2514142, ERR2514143, ERR2514144, ERR2514145, ERR2514146, ERR2514147, ERR2514148, ERR2512408, ERR2514149, ERR2514150, ERR2514151, ERR2514152, ERR2514153, ERR2513017, ERR2514154, ERR2514155, ERR2514156, ERR2514157, ERR2514158, ERR2514159, ERR2514160, ERR2514161, ERR2512415, ERR2514162, ERR2514163, ERR2512623, ERR2514164, ERR2514165, ERR2514166, ERR2514167, ERR2514168, ERR2514169, ERR2514170, ERR2514171, ERR2514172, ERR2514173, ERR2514174, ERR2513073, ERR2514175, ERR2514176, ERR2514177, ERR2514179, ERR2514180, ERR2514181, ERR2514182, ERR2514183, ERR2514184, ERR2514185, ERR2514186, ERR2514187, ERR2514188, ERR2514189, ERR2514190, ERR2514191, ERR2514192, ERR2514193, ERR2514194, ERR2514195, ERR2514196, ERR2514197, ERR2514198, ERR2514199, ERR2514200, ERR2514201, ERR2514202, ERR2514203, ERR2514204, ERR2514205, ERR2514206, ERR2514207, ERR2514208, ERR2514209, ERR2514211, ERR2514212, ERR2514213, ERR2514214, ERR2514215, ERR2513853, ERR2514216, ERR2514217, ERR2514218, ERR2514219, ERR2514221, ERR2514222, ERR2514223, ERR2514224, ERR2514225, ERR2512984, ERR2514226, ERR2514227, ERR2514228, ERR2514229, ERR2514231, ERR2514232, ERR2514233, ERR2514234, ERR2514235, ERR2512663, ERR2514236, ERR2514237, ERR2514238, ERR2514239, ERR2514240, ERR2514241, ERR2514242, ERR2514243, ERR2514244, ERR2513021, ERR2514245, ERR2514246, ERR2514247, ERR2514248, ERR2514249, ERR2514250, ERR2514251, ERR2514252, ERR2514253, ERR2514254, ERR2514255, ERR2514256, ERR2514257, ERR2514258, ERR2512428, ERR2514259, ERR2514260, ERR2514261, ERR2514262, ERR2514263, ERR2514264, ERR2514265, ERR2514266, ERR2514267, ERR2514268, ERR2514269, ERR2514270, ERR2514271, ERR2514272, ERR2514273, ERR2514274, ERR2514276, ERR2514277, ERR2514278, ERR2514279, ERR2514280, ERR2514281, ERR2514282, ERR2514283, ERR2514284, ERR2514285, ERR2514286, ERR2514287, ERR2514288, ERR2514289, ERR2514290, ERR2514291, ERR2514292, ERR2514293, ERR2514294, ERR2514295, ERR2514296, ERR2514297, ERR2514298, ERR2514299, ERR2514300, ERR2514301, ERR2514302, ERR2514303, ERR2514304, ERR2514305, ERR2514306, ERR2514307, ERR2514308, ERR2514309, ERR2514310, ERR2514311, ERR2514312, ERR2514313, ERR2514314, ERR2514315, ERR2514316, ERR2514317, ERR2514318, ERR2514319, ERR2514320, ERR2514321, ERR2514322, ERR2514323, ERR2514324, ERR2514325, ERR2514326, ERR2514327, ERR2514328, ERR2514329, ERR2514331, ERR2514332, ERR2514333, ERR2514334, ERR2514335, ERR2514336, ERR2514337, ERR2514338, ERR2514339, ERR2512420, ERR2514340, ERR2514341, ERR2514342, ERR2514343, ERR2514344, ERR2514345, ERR2514346, ERR2514347, ERR2514348, ERR2514349, ERR2514351, ERR2514352, ERR2514353, ERR2514355, ERR2514356, ERR2512589, ERR2514357, ERR2514358, ERR2514359, ERR2514360, ERR2514361, ERR2514363, ERR2514364, ERR2514365, ERR2514366, ERR2514367, ERR2514368, ERR2514369, ERR2514370, ERR2514371, ERR2514372, ERR2514373, ERR2514374, ERR2514375, ERR2514376, ERR2514377, ERR2514378, ERR2514380, ERR2514381, ERR2514382, ERR2514383, ERR2514384, ERR2514385, ERR2514386, ERR2514387, ERR2514389, ERR2514391, ERR2514392, ERR2514393, ERR2514394, ERR2514395, ERR2514396, ERR2514397, ERR2514399, ERR2514400, ERR2514401, ERR2514402, ERR2514403, ERR2514404, ERR2514405, ERR2514406, ERR2514407, ERR2513835, ERR2514408, ERR2514409, ERR2514410, ERR2514411, ERR2514412, ERR2514413, ERR2514414, ERR2514415, ERR2514416, ERR2514417, ERR2514418, ERR2514419, ERR2514420, ERR2514421, ERR2514422, ERR2514423, ERR2514424, ERR2514425, ERR2514426, ERR2514427, ERR2514428, ERR2514429, ERR2514430, ERR2514431, ERR2514432, ERR2514433, ERR2514434, ERR2514435, ERR2514436, ERR2514437, ERR2514438, ERR2514439, ERR2514440, ERR2514441, ERR2514442, ERR2514443, ERR2514444, ERR2514445, ERR2514446, ERR2514447, ERR2514448, ERR2514449, ERR2514450, ERR2514451, ERR2514452, ERR2514453, ERR2514454, ERR2514455, ERR2514456, ERR2514457, ERR2514458, ERR2514459, ERR2514460, ERR2514461, ERR2514462, ERR2514463, ERR2514464, ERR2514465, ERR2514466, ERR2514467, ERR2514468, ERR2514469, ERR2514470, ERR2514471, ERR2514472, ERR2512701, ERR2514473, ERR2514474, ERR2514475, ERR2514476, ERR2514477, ERR2514478, ERR2514479, ERR2514480, ERR2514481, ERR2514482, ERR2514483, ERR2514484, ERR2514485, ERR2514486, ERR2514487, ERR2514489, ERR2514490, ERR2514491, ERR2514492, ERR2514493, ERR2514494, ERR2514495, ERR2514496, ERR2514497, ERR2514498, ERR2514499, ERR2514500, ERR2514501, ERR2514502, ERR2514503, ERR2514504, ERR2514505, ERR2514506, ERR2513522, ERR2514507, ERR2514508, ERR2514509, ERR2514510, ERR2514511, ERR2514512, ERR2514513, ERR2514515, ERR2514516, ERR2514517, ERR2514518, ERR2512403, ERR2514519, ERR2514520, ERR2514521, ERR2514523, ERR2514524, ERR2514525, ERR2514526, ERR2514527, ERR2514528, ERR2514529, ERR2514530, ERR2513367, ERR2514531, ERR2514532, ERR2514533, ERR2514534, ERR2514536, ERR2514537, ERR2514538, ERR2514539, ERR2514540, ERR2514541, ERR2514542, ERR2514543, ERR2514544, ERR2514545, ERR2514546, ERR2514547, ERR2514549, ERR2514550, ERR2514551, ERR2514552, ERR2514553, ERR2514554, ERR2514555, ERR2514556, ERR2512459, ERR2514558, ERR2514560, ERR2514561, ERR2514562, ERR2514563, ERR2514564, ERR2514565, ERR2514566, ERR2514567, ERR2514568, ERR2514569, ERR2514570, ERR2513562, ERR2514571, ERR2514572, ERR2514573, ERR2514574, ERR2514575, ERR2514576, ERR2514577, ERR2514578, ERR2514579, ERR2514580, ERR2514581, ERR2514582, ERR2514583, ERR2514584, ERR2514585, ERR2514586, ERR2514587, ERR2514588, ERR2514589, ERR2514590, ERR2514592, ERR2514593, ERR2514594, ERR2514595, ERR2514596, ERR2514597, ERR2514598, ERR2514600, ERR2514601, ERR2514602, ERR2514603, ERR2514604, ERR2514605, ERR2514606, ERR2514607, ERR2514608, ERR2514609, ERR2514610, ERR2514611, ERR2514612, ERR2514613, ERR2514614, ERR2514615, ERR2514616, ERR2514617, ERR2514618, ERR2514619, ERR2514620, ERR2514621, ERR2514622, ERR2514624, ERR2514625, ERR2514626, ERR2514627, ERR2514628, ERR2514629, ERR2514630, ERR2514631, ERR2514632, ERR2514633, ERR2514634, ERR2514635, ERR2514636, ERR2514637, ERR2514638, ERR2514639, ERR2514640, ERR2514641, ERR2514642, ERR2514643, ERR2514644, ERR2514645, ERR2514647, ERR2514648, ERR2514649, ERR2514650, ERR2514651, ERR2514652, ERR2514653, ERR2514654, ERR2514655, ERR2514656, ERR2514657, ERR2514658, ERR2514659, ERR2514660, ERR2514661, ERR2514662, ERR2514663, ERR2514664, ERR2514665, ERR2514666, ERR2514667, ERR2514668, ERR2514669, ERR2514670, ERR2514671, ERR2514672, ERR2514673, ERR2514674, ERR2514675, ERR2514676, ERR2514677, ERR2514678, ERR2514679, ERR2514680, ERR2514681, ERR2514682, ERR2514683, ERR2514684, ERR2514685, ERR2514686, ERR2514687, ERR2514690, ERR2514691, ERR2514693, ERR2514694, ERR2514696, ERR2514697, ERR2514698, ERR2514699, ERR2514700, ERR2514701, ERR2514702, ERR2514703, ERR2514704, ERR2512393, ERR2514705, ERR2514706, ERR2514707, ERR2514709, ERR2514710, ERR2514711, ERR2512869, ERR2514712, ERR2514713, ERR2514714, ERR2514715, ERR2514716, ERR2514717, ERR2514718, ERR2514719, ERR2514720, ERR2514721, ERR2514722, ERR2514723, ERR2514724, ERR2513400, ERR2512400, ERR2514725, ERR2514726, ERR2514727, ERR2514728, ERR2514729, ERR2514730, ERR2514731, ERR2514732, ERR2514734, ERR2514736, ERR2514737, ERR2514738, ERR2514739, ERR2514741, ERR2514742, ERR2514743, ERR2514744, ERR2514745, ERR2514746, ERR2514747, ERR2514748, ERR2514749, ERR2514750, ERR2514751, ERR2514752, ERR2514753, ERR2514754, ERR2514755, ERR2514756, ERR2514757, ERR2514758, ERR2514759, ERR2514760, ERR2514761, ERR2514762, ERR2514763, ERR2514764, ERR2514765, ERR2514766, ERR2514768, ERR2514769, ERR2514770, ERR2514771, ERR2514772, ERR2514773, ERR2514774, ERR2514775, ERR2514776, ERR2514777, ERR2512697, ERR2514778, ERR2514780, ERR2514781, ERR2514782, ERR2514783, ERR2514784, ERR2514785, ERR2514786, ERR2514787, ERR2514788, ERR2514789, ERR2514790, ERR2514791, ERR2513314, ERR2514792, ERR2514793, ERR2514794, ERR2514795, ERR2514796, ERR2514797, ERR2514798, ERR2514799, ERR2514800, ERR2514801, ERR2514802, ERR2514803, ERR2514804, ERR2514805, ERR2514807, ERR2514808, ERR2514809, ERR2514810, ERR2514811, ERR2514812, ERR2514813, ERR2514814, ERR2514816, ERR2514817, ERR2514818, ERR2514819, ERR2514820, ERR2514821, ERR2514823, ERR2514824, ERR2514825, ERR2514826, ERR2514827, ERR2514828, ERR2514829, ERR2514830, ERR2514831, ERR2514832, ERR2514833, ERR2514834, ERR2514835, ERR2514836, ERR2514837, ERR2514838, ERR2514839, ERR2514840, ERR2514841, ERR2514842, ERR2514843, ERR2514844, ERR2514845, ERR2514846, ERR2514847, ERR2514848, ERR2514849, ERR2514850, ERR2514851, ERR2514852, ERR2514853, ERR2514854, ERR2514855, ERR2514856, ERR2514857, ERR2514858, ERR2514860, ERR2514861, ERR2514862, ERR2514863, ERR2514865, ERR2514866, ERR2514868, ERR2514869, ERR2514870, ERR2514871, ERR2514872, ERR2514873, ERR2514874, ERR2514875, ERR2514876, ERR2512703, ERR2514878, ERR2514879, ERR2514880, ERR2512488, ERR2514881, ERR2514882, ERR2514883, ERR2514884, ERR2514885, ERR2514886, ERR2514887, ERR2514888, ERR2514889, ERR2514890, ERR2514891, ERR2514892, ERR2514893, ERR2514894, ERR2514895, ERR2514896, ERR2514897, ERR2514898, ERR2514899, ERR2514900, ERR2514901, ERR2514902, ERR2514903, ERR2514904, ERR2514905, ERR2514906, ERR2514907, ERR2514908, ERR2514909, ERR2514910, ERR2514911, ERR2514912, ERR2514913, ERR2514914, ERR2514915, ERR2514916, ERR2514917, ERR2514918, ERR2514919, ERR2514920, ERR2514921, ERR2514922, ERR2514923, ERR2514924, ERR2514925, ERR2514926, ERR2514927, ERR2514928, ERR2514929, ERR2514930, ERR2514931, ERR2512721, ERR2514932, ERR2514933, ERR2514934, ERR2514935, ERR2514936, ERR2514937, ERR2514938, ERR2514939, ERR2514940, ERR2514941, ERR2514942, ERR2514943, ERR2514944, ERR2514945, ERR2514946, ERR2514947, ERR2514948, ERR2514949, ERR2514950, ERR2514951, ERR2514953, ERR2514954, ERR2514955, ERR2514956, ERR2514957, ERR2514958, ERR2514959, ERR2514960, ERR2514961, ERR2514963, ERR2514964, ERR2514965, ERR2514966, ERR2514967, ERR2514968, ERR2514969, ERR2514970, ERR2514971, ERR2514972, ERR2514973, ERR2514974, ERR2514975, ERR2514976, ERR2514977, ERR2514978, ERR2514980, ERR2514982, ERR2514983, ERR2514984, ERR2514985, ERR2514986, ERR2512641, ERR2514987, ERR2514989, ERR2514990, ERR2514991, ERR2514992, ERR2514993, ERR2514994, ERR2514995, ERR2514997, ERR2514998, ERR2514999, ERR2515000, ERR2515001, ERR2515002, ERR2515003, ERR2515004, ERR2515005, ERR2515006, ERR2515007, ERR2515008, ERR2515009, ERR2515010, ERR2515011, ERR2515012, ERR2515013, ERR2512516, ERR2515015, ERR2515016, ERR2515017, ERR2515018, ERR2515019, ERR2515020, ERR2515021, ERR2515022, ERR2515023, ERR2515024, ERR2515025, ERR2515026, ERR2515027, ERR2515028, ERR2515029, ERR2515030, ERR2515031, ERR2513421, ERR2513707, ERR2515032, ERR2515033, ERR2515034, ERR2515036, ERR2515037, ERR2513271, ERR2515038, ERR2515039, ERR2515040, ERR2515041, ERR2515042, ERR2515043, ERR2515044, ERR2515045, ERR2515046, ERR2515047, ERR2515048, ERR2515049, ERR2515050, ERR2515051, ERR2515052, ERR2515053, ERR2515054, ERR2515055, ERR2515056, ERR2515057, ERR2515058, ERR2515059, ERR2515060, ERR2515061, ERR2515062, ERR2515063, ERR2513643, ERR2515065, ERR2515066, ERR2515067, ERR2515068, ERR2515069, ERR2512533, ERR2515070, ERR2515071, ERR2515072, ERR2515073, ERR2515074, ERR2515075, ERR2515076, ERR2515077, ERR2515078, ERR2515079, ERR2515080, ERR2515081, ERR2515082, ERR2515083, ERR2513829, ERR2515084, ERR2515085, ERR2515086, ERR2515088, ERR2515089, ERR2515090, ERR2515092, ERR2515093, ERR2512387, ERR2515094, ERR2515095, ERR2515096, ERR2515097, ERR2515098, ERR2515099, ERR2515100, ERR2515101, ERR2515102, ERR144542, ERR234667, ERR067679, ERR067625, ERR067719, ERR227988, ERR067737, ERR067585, ERR234564, ERR067619, ERR108422, ERR067676, ERR067586, ERR067583, ERR133804, ERR067732, ERR067587, ERR067590, ERR133907, ERR137271, ERR067682, ERR067753, ERR067736, ERR108444, ERR144613, ERR067663, ERR067636, ERR067604, ERR067664, ERR067576, ERR067601, ERR144618, ERR234695, ERR133893, ERR133808, ERR137199, ERR067714, ERR137205, ERR067582, ERR144557, ERR067657, ERR067707, ERR2516166, ERR2516167, ERR2516168, ERR2516169, ERR2516170, ERR2516171, ERR2516172, ERR2516173, ERR2516174, ERR2516175, ERR2516177, ERR2516178, ERR2516179, ERR2516180, ERR2516181, ERR2516182, ERR2516183, ERR2516184, ERR2516185, ERR2516186, ERR2516187, ERR2516188, ERR2516189, ERR2516191, ERR2516192, ERR2516193, ERR2516194, ERR2516195, ERR2516196, ERR2516197, ERR2516198, ERR2516199, ERR2516200, ERR2516201, ERR2516202, ERR2516203, ERR2516204, ERR2516205, ERR2516206, ERR2516207, ERR2516208, ERR2516209, ERR2516210, ERR2516211, ERR2516212, ERR2516213, ERR2516214, ERR2516215, ERR2516216, ERR2516217, ERR2516218, ERR2516219, ERR2516220, ERR2516221, ERR2516222, ERR2516223, ERR2516224, ERR2516225, ERR2516226, ERR2516227, ERR2516228, ERR2516230, ERR2516231, ERR2516233, ERR2516234, ERR2516235, ERR2516236, ERR2516238, ERR2516239, ERR2516240, ERR2516241, ERR2516242, ERR2516243, ERR2516244, ERR2516245, ERR2516246, ERR2516247, ERR2516248, ERR2516250, ERR2516252, ERR2516253, ERR2516254, ERR2516255, ERR2516256, ERR2516257, ERR2516258, ERR2516259, ERR2516260, ERR2516261, ERR2516262, ERR2516263, ERR2516264, ERR2516265, ERR2516266, ERR2516267, ERR2516268, ERR2516269, ERR2516270, ERR2516271, ERR2516272, ERR2516273, ERR2516274, ERR2516275, ERR2516276, ERR2516277, ERR2516278, ERR2516279, ERR2516281, ERR2516283, ERR2516284, ERR2516285, ERR2516286, ERR2516287, ERR2516288, ERR2516289, ERR2516290, ERR2516291, ERR2516292, ERR2516293, ERR2516294, ERR2516295, ERR2516296, ERR2516298, ERR2516299, ERR2516300, ERR2516301, ERR2516302, ERR2516303, ERR2516304, ERR2516305, ERR2516306, ERR2516307, ERR2516308, ERR2516309, ERR2516310, ERR2516311, ERR2516312, ERR2516313, ERR2516314, ERR2516315, ERR2516316, ERR2516317, ERR2516318, ERR2516319, ERR2516321, ERR2516322, ERR2516323, ERR2516324, ERR2516325, ERR2516326, ERR2516327, ERR2516328, ERR2516329, ERR2516330, ERR2516331, ERR2516332, ERR2516333, ERR2516334, ERR2516335, ERR2516336, ERR2516337, ERR2516338, ERR2516339, ERR2516340, ERR2516341, ERR2516342, ERR2516343, ERR2516345, ERR2516346, ERR2516347, ERR2516348, ERR2516349, ERR2516350, ERR2516351, ERR2516352, ERR2516353, ERR2516354, ERR2516355, ERR2516356, ERR2516357, ERR2516358, ERR2516360, ERR2516361, ERR2516362, ERR2516363, ERR2516364, ERR2516365, ERR2516366, ERR2516367, ERR2516368, ERR2516369, ERR2516370, ERR2516371, ERR2516372, ERR2516373, ERR2516374, ERR2516375, ERR2516376, ERR2516377, ERR2516378, ERR2516379, ERR2516380, ERR2516381, ERR2516382, ERR2516383, ERR2516384, ERR2516385, ERR2516386, ERR2516387, ERR2516388, ERR2516389, ERR2516391, ERR2516392, ERR2516393, ERR2516394, ERR2516395, ERR2516396, ERR2516397, ERR2516398, ERR2516399, ERR2199821, ERR2199781, ERR2199746, ERR551572, ERR2200140, ERR2199963, ERR551585, ERR552333, ERR2199763, ERR2200012, ERR2199915, ERR551675, ERR2199849, ERR2200129, ERR2199863, ERR551288, ERR2199912, ERR2199783, ERR2199822, ERR551866, ERR551112, ERR2199797, ERR2199743, ERR2199779, ERR2200106, ERR2199817, ERR2200141, ERR2200081, ERR2199881, ERR2200089, ERR2199985, ERR2199893, ERR2199904, ERR2199846, ERR2199887, ERR2199962, ERR2199798, ERR2200151, ERR2200036, ERR2200124, ERR2200138, ERR553293, ERR2200132, ERR2199847, ERR2199964, ERR1768629, ERR2199773, ERR551316, ERR498359, ERR2199905, ERR552252, ERR2200123, ERR2200116, ERR2199917, ERR2200109, ERR498289, ERR498369, ERR2199871, ERR2200127, ERR2199865, ERR498365, ERR2200029, ERR2199859, ERR2199900, ERR552667, ERR2200144, ERR2199948, ERR2200062, ERR553385, ERR550899, ERR2199801, ERR498355, ERR2200082, ERR2200104, ERR2200102, ERR2199800, ERR2199841, ERR2199870, ERR2200111, ERR2200050, ERR2199911, ERR2199903, ERR2200042, ERR2200149, ERR2200145, ERR2200143, ERR2199782, ERR551318, ERR2200112, ERR2200073, ERR2199812, ERR2199883, ERR2199918, ERR2199819, ERR2199874, ERR2200136, ERR2199864, ERR2199838, ERR2199906, ERR2199974, ERR2199987, ERR553125, ERR552755, ERR2199891, ERR2199776, ERR552314, ERR2199873, ERR2199765, ERR2200027, ERR2200099, ERR550640, ERR2199745, ERR551265, ERR2199868, ERR2199978, ERR550739, ERR2199960, ERR551471, ERR2199769, ERR2199827, ERR2200063, ERR2199888, ERR550998, ERR553278, ERR2199965, ERR2199967, ERR2199840, ERR552784, ERR2200054, ERR2200028, ERR2200041, ERR2199892, ERR2199861, ERR2199886, ERR2199804, ERR2199780, ERR2199756, ERR551853, ERR2199884, ERR553291, ERR2199761, ERR552469, ERR550718, ERR2200108, ERR498366, ERR2199796, ERR2199896, ERR2199741, ERR498356, ERR2199898, ERR2199785, ERR2200120, ERR550701, ERR550624, ERR498286, ERR2199820, ERR2199788, ERR2199831, ERR551882, ERR2200100, ERR2200110, ERR2199832, ERR2199829, ERR553277, ERR2199791, ERR498352, ERR2199806, ERR2199890, ERR2200059, ERR2199850, ERR2199839, ERR2199851, ERR2199895, ERR553082, ERR498362, ERR2200113, ERR2199879, ERR2200090, ERR2199958, ERR2200085, ERR552959, ERR2199857, ERR551086, ERR2199966, ERR2199833, ERR2199830, ERR2199860, ERR2200101, ERR2199794, ERR2199786, ERR2199845, ERR1768638, ERR2200086, ERR2199877, ERR2199986, ERR2199916, ERR2199875, ERR551694, ERR551928, ERR2199902, ERR2199913, ERR552690, ERR2199835, ERR551978, ERR2199770, ERR2200130, ERR2200121, ERR2199909, ERR2199852, ERR2199784, ERR2199795, ERR2200055, ERR2199957, ERR2199823, ERR2200016, ERR2200131, ERR2199803, ERR2200072, ERR498299, ERR553215, ERR2199754, ERR2199983, ERR2199878, ERR2200084, ERR2200003, ERR2200147, ERR2199816, ERR2199961, ERR2199772, ERR498384, ERR2199858, ERR2200091, ERR2199809, ERR2199854, ERR2199792, ERR2199753, ERR2199867, ERR2199799, ERR2199848, ERR2200133, ERR551666, ERR2200107, ERR2199789, ERR2200053, ERR498295, ERR2200097, ERR2200114, ERR2199889, ERR2199744, ERR2199828, ERR2199742, ERR2200057, ERR2199837, ERR551123, ERR552284, ERR2200125, ERR2199808, ERR2200126, ERR2199919, ERR2199825, ERR2199818, ERR2200134, ERR550927, ERR2199897, ERR2199894, ERR551401, ERR2200071, ERR2199980, ERR2199856, ERR2199810, ERR2199908, ERR2199777, ERR2200056, ERR2516400, ERR2516403, ERR2516404, ERR2516405, ERR2516407, ERR2516408, ERR2516409, ERR2516410, ERR2516411, ERR2516412, ERR2516413, ERR2516414, ERR2516417, ERR2516418, ERR2516419, ERR2516420, ERR2516421, ERR2516422, ERR2516423, ERR2516424, ERR2516425, ERR2516426, ERR2516427, ERR2516428, ERR2516429, ERR2516430, ERR2516431, ERR2516433, ERR2516434, ERR2516436, ERR2516437, ERR2516440, ERR2516441, ERR2516442, ERR2516443, ERR2516445, ERR2516446, ERR2516447, ERR2516448, ERR2516449, ERR2516450, ERR2516451, ERR2516452, ERR2516455, ERR2516457, ERR2516458, ERR2516459, ERR2516460, ERR2516461, ERR2516462, ERR2516463, ERR2516464, ERR2516465, ERR2516467, ERR2516468, ERR2516472, ERR2516473, ERR2516474, ERR2516475, ERR2516476, ERR2516477, ERR2516478, ERR2516479, ERR2516480, ERR2516481, ERR2516482, ERR2516484, ERR2516485, ERR2516486, ERR2516487, ERR2516488, ERR2516489, ERR2516490, ERR2516491, ERR2516492, ERR2516493, ERR2516494, ERR2516495, ERR2516496, ERR2516497, ERR2516498, ERR2516499, ERR2516500, ERR2516501, ERR2516503, ERR2516504, ERR2516505, ERR2516506, ERR2516507, ERR2516508, ERR2516509, ERR2516511, ERR2516513, ERR2516514, ERR2516515, ERR2516517, ERR2516518, ERR2516519, ERR2516520, ERR2516522, ERR2516523, ERR2516524, ERR2516525, ERR2516526, ERR2516527, ERR2516528, ERR2516529, ERR2516531, ERR2516532, ERR2516533, ERR2516534, ERR2516535, ERR2516536, ERR2516537, ERR2516538, ERR2516539, ERR2516540, ERR2516541, ERR2516542, ERR2516543, ERR2516544, ERR2516545, ERR2516546, ERR2516547, ERR2516548, ERR2516549, ERR2516551, ERR2516552, ERR2516554, ERR2516556, ERR2516557, ERR2516558, ERR2516560, ERR2516561, ERR2516562, ERR2516564, ERR2516565, ERR2516566, ERR2516567, ERR2516568, ERR2516570, ERR2516572, ERR2516573, ERR2516574, ERR2516577, ERR2516578, ERR2516581, ERR2516583, ERR2516584, ERR2516586, ERR2516587, ERR2516589, ERR2516590, ERR2516593, ERR2516594, ERR2516595, ERR2516596, ERR2516597, ERR2516598, ERR2516599, ERR2516601, ERR2516602, ERR2516603, ERR2516604, ERR2516605, ERR2516607, ERR2516608, ERR2516609, ERR2516610, ERR2516611, ERR2516612, ERR2516614, ERR2516615, ERR2516616, ERR2516618, ERR2516619, ERR2516620, ERR2516622, ERR2516623, ERR2516624, ERR2516625, ERR2516627, ERR2516628, ERR2516629, ERR2516630, ERR2516631, ERR2516632, ERR2516634, ERR2516636, ERR2516637, ERR2516638, ERR2516639, ERR2516641, ERR2516642, ERR2516643, ERR2516644, ERR2516645, ERR2516646, ERR2516647, ERR2516648, ERR2516650, ERR2516653, ERR2516655, ERR2516656, ERR2516657, ERR2516658, ERR2516660, ERR2516661, ERR2516662, ERR2516664, ERR2516665, ERR2516666, ERR2516667, ERR2516668, ERR2516669, ERR2516670, ERR2516671, ERR2516672, ERR2516673, ERR2516674, ERR2516675, ERR2516676, ERR2516678, ERR2516680, ERR2516681, ERR2516682, ERR2516683, ERR2516685, ERR2516686, ERR2516687, ERR2516689, ERR2516691, ERR2516692, ERR2516693, ERR2516694, ERR2516695, ERR2516696, ERR2516697, ERR2516698, ERR2516699, ERR2516700, ERR2516702, ERR2516703, ERR2516704, ERR2516705, ERR2516706, ERR2516708, ERR2516709, ERR2516710, ERR2516711, ERR2516714, ERR2516715, ERR2516716, ERR2516717, ERR2516718, ERR2516721, ERR2516722, ERR2516723, ERR2516724, ERR2516727, ERR2516728, ERR2516729, ERR2516730, ERR2516731, ERR2516732, ERR2516733, ERR2516734, ERR2516735, ERR2516737, ERR2516738, ERR2510880, ERR2510858, ERR2510878, ERR2510725, ERR2510459, ERR2510254, ERR2510713, ERR2510668, ERR2510678, ERR2510692, ERR2510666, ERR2510677, ERR2510843, ERR2510683, ERR2510732, ERR2510847, ERR2510662, ERR2510715, ERR2510329, ERR2510726, ERR2510864, ERR2510850, ERR2510659, ERR2510723, ERR2510674, ERR2510735, ERR2510660, ERR2510708, ERR2510710, ERR2510729, ERR2510866, ERR2510867, ERR2510689, ERR2510672, ERR2510687, ERR2510703, ERR2510851, ERR2510360, ERR2510663, ERR2510718, ERR2510834, ERR2510691, ERR2510675, ERR2510728, ERR2510730, ERR2510681, ERR2510711, ERR2510665, ERR2510842, ERR2510855, ERR2510716, ERR2510854, ERR2510706, ERR2510702, ERR2510712, ERR2510863, ERR2510673, ERR2510714, ERR2510827, ERR2510846, ERR2510881, ERR2510510, ERR2510664, ERR2510840, ERR2510704, ERR2510709, ERR2510688, ERR2510654, ERR2510832, ERR2510882, ERR2510872, ERR2510852, ERR2510853, ERR2510676, ERR2510823, ERR2510679, ERR2510693, ERR2510658, ERR2510705, ERR2510690, ERR2510682, ERR2510671, ERR2510845, ERR2510686, ERR2510721, ERR2510856, ERR2510695, ERR2510839, ERR2510849, ERR2510859, ERR2510731, ERR2510720, ERR2510848, ERR2510680, ERR2510862, ERR2510734, ERR2510279, ERR2510685, ERR2510868, ERR2510833, ERR2510684, ERR2510719, ERR2510653, ERR2510696, ERR2510876, ERR2510733, ERR2510841, ERR2510865, ERR2510670, ERR2510707, ERR2510717, ERR2510826, ERR2510669, ERR2510857, ERR2510844, ERR2510694, ERR2510661, ERR2510778, ERR2510750, ERR2510781, ERR2510812, ERR2510768, ERR2510821, ERR2510831, ERR2510798, ERR2510811, ERR2510810, ERR2510780, ERR2510755, ERR2510759, ERR2510817, ERR2510747, ERR2510828, ERR2510830, ERR2510835, ERR2510808, ERR2510782, ERR2510756, ERR2510760, ERR2510754, ERR2510822, ERR2510825, ERR2510820, ERR2510795, ERR2510784, ERR2510736, ERR2510797, ERR2510809, ERR2510786, ERR2510766, ERR2510752, ERR2510774, ERR2510758, ERR2510804, ERR2510802, ERR2510745, ERR2510775, ERR2510772, ERR2510787, ERR2510815, ERR2510764, ERR2510789, ERR2510779, ERR2510792, ERR2510742, ERR2510799, ERR2510765, ERR2510769, ERR2510819, ERR2510813, ERR2510770, ERR2510738, ERR2510788, ERR2510836, ERR2510763, ERR2510783, ERR2510740, ERR2510814, ERR2510767, ERR2510805, ERR2510800, ERR2510741, ERR2510757, ERR2510807, ERR2510777, ERR2510801, ERR2510739, ERR2510773, ERR2510816, ERR2510737, ERR2510748, ERR2510803, ERR2510791, ERR2510744, ERR2510749, ERR2510829, ERR2510776, ERR2510796, ERR2510761, ERR2510771, ERR2510746, ERR2510794, ERR2510751, ERR2510793, ERR2510818, ERR2510753, ERR2510785, ERR2510837, ERR2510743, ERR2510790, ERR2510806, ERR2516740, ERR2516741, ERR2516742, ERR2516745, ERR2516746, ERR2516748, ERR2516750, ERR2516753, ERR2516754, ERR2516755, ERR2516757, ERR2516758, ERR2516767, ERR2516774, ERR2516776, ERR2516779, ERR2516781, ERR2516782, ERR2516783, ERR2516784, ERR2516786, ERR2516787, ERR2516788, ERR2516789, ERR2516792, ERR2516793, ERR2516795, ERR2516796, ERR2516798, ERR2516799, ERR2516801, ERR2516804, ERR2516805, ERR2516808, ERR2516809, ERR2516810, ERR2516811, ERR2516812, ERR2516814, ERR2516817, ERR2516818, ERR2516819, ERR2516820, ERR2516821, ERR2516822, ERR2516823, ERR2516824, ERR2516825, ERR2516827, ERR2516829, ERR2516830, ERR2516832, ERR2516834, ERR2516841, ERR2516843, ERR2516844, ERR2516845, ERR2516846, ERR2516854, ERR2516855, ERR2516856, ERR2516859, ERR2516860, ERR2516863, ERR2516868, ERR2516869, ERR2516870, ERR2516872, ERR2516873, ERR2516875, ERR2516878, ERR2516879, ERR2516884, ERR2516886, ERR2516888, ERR2516891, ERR2516896, ERR2516898, ERR2516899, ERR2516900, ERR2516903, ERR2516904, ERR2516909, ERR2516910, ERR2516911, ERR2516912, ERR2516913, ERR2516914, ERR2516916, ERR2516918, ERR2516919, ERR2516923, ERR2516924, ERR2516925, ERR2516926, ERR2516928, ERR2516929, ERR2516930, ERR2516933, ERR2516934, ERR2516935, ERR2516937, ERR2516939, ERR2516941, ERR2516943, ERR2516944, ERR2516948, ERR2516950, ERR2516952, ERR2516953, ERR2516959, ERR2516960, ERR2516962, ERR2516963, ERR2516964, ERR2516965, ERR2516966, ERR2516968, ERR2516970, ERR2516971, ERR2516972, ERR2516973, ERR2516975, ERR2516976, ERR2516980, ERR2516981, ERR2516982, ERR2516985, ERR2516987, ERR2516988, ERR2516991, ERR2516992, ERR2516993, ERR2516994, ERR2516998, ERR2517000, ERR2517004, ERR2517005, ERR2517007, ERR2517008, ERR2517011, ERR2517012, ERR2517013, ERR2517014, ERR2517016, ERR2517018, ERR2517019, ERR2517021, ERR2517022, ERR2517023, ERR2517025, ERR2517026, ERR2517027, ERR2517028, ERR2517029, ERR2517030, ERR2517031, ERR2517032, ERR2517034, ERR2517035, ERR2517036, ERR2517037, ERR2517039, ERR2517042, ERR2517043, ERR2517047, ERR2517050, ERR2517051, ERR2517053, ERR2517058, ERR2517059, ERR2517060, ERR2517061, ERR2517063, ERR2517066, ERR2517068, ERR2517069, ERR2517070, ERR2517072, ERR2517074, ERR2517078, ERR2517079, ERR2517082, ERR2517085, ERR2517088, ERR2517089, ERR2517090, ERR2517091, ERR2517092, ERR2517094, ERR2517102, ERR2517103, ERR2517104, ERR2517105, ERR2517107, ERR2517108, ERR2517109, ERR2517110, ERR2517111, ERR2517115, ERR2517118, ERR2517120, ERR2517121, ERR2517122, ERR2517123, ERR2517124, ERR553021, ERR552491, ERR551231, ERR551650, ERR552573, ERR552420, ERR552789, ERR551969, ERR553204, ERR552221, ERR498238, ERR552763, ERR553357, ERR551523, ERR552010, ERR552754, ERR552765, ERR553354, ERR552612, ERR553350, ERR552115, ERR553182, ERR552254, ERR552710, ERR552981, ERR552878, ERR551545, ERR551739, ERR552000, ERR552405, ERR551299, ERR550864, ERR550657, ERR550861, ERR553241, ERR552992, ERR552672, ERR552415, ERR553053, ERR551536, ERR552929, ERR551621, ERR552151, ERR551045, ERR553134, ERR551692, ERR551280, ERR551242, ERR551758, ERR552124, ERR551456, ERR552071, ERR550872, ERR553058, ERR552193, ERR552487, ERR553328, ERR551391, ERR552495, ERR553230, ERR553221, ERR551166, ERR552288, ERR551920, ERR550741, ERR551953, ERR552073, ERR551488, ERR552150, ERR551289, ERR550794, ERR552987, ERR553366, ERR550869, ERR552695, ERR551689, ERR553002, ERR551801, ERR551329, ERR551204, ERR550976, ERR552823, ERR553321, ERR553242, ERR551014, ERR552273, ERR553358, ERR553313, ERR552745, ERR552701, ERR552540, ERR551319, ERR551938, ERR551578, ERR552456, ERR552077, ERR550619, ERR551537, ERR553059, ERR550754, ERR550636, ERR551899, ERR552809, ERR552496, ERR552518, ERR552994, ERR553216, ERR553284, ERR552599, ERR550734, ERR552463, ERR552352, ERR552070, ERR553116, ERR551896, ERR552484, ERR553282, ERR2517580, ERR2517140, ERR2517159, ERR2517260, ERR2517161, ERR2517568, ERR2517334, ERR2517154, ERR2517408, ERR2517297, ERR2517167, ERR2517428, ERR2517599, ERR2517399, ERR2517127, ERR2517494, ERR2517235, ERR2517487, ERR2517578, ERR2517411, ERR2517269, ERR2517194, ERR2517598, ERR2517577, ERR2517333, ERR2517300, ERR2517374, ERR2517253, ERR2517358, ERR2517589, ERR2517564, ERR2517600, ERR2517183, ERR2517245, ERR2517460, ERR2517153, ERR2517202, ERR2517470, ERR2517165, ERR2517278, ERR2517354, ERR2517440, ERR2517155, ERR2517336, ERR2517455, ERR2517215, ERR2517591, ERR2517590, ERR2517344, ERR2517179, ERR2517498, ERR2517217, ERR2517178, ERR2517492, ERR2517239, ERR2517276, ERR2517287, ERR2517319, ERR2517174, ERR2517552, ERR2517433, ERR2517184, ERR2517182, ERR2517148, ERR2517341, ERR2517226, ERR2517373, ERR2517559, ERR2517464, ERR2517384, ERR2517643, ERR2517497, ERR2517258, ERR2517199, ERR2517220, ERR2517485, ERR2517412, ERR2517221, ERR2517335, ERR2517201, ERR2517192, ERR2517604, ERR2517594, ERR2517618, ERR2517410, ERR2517615, ERR2517205, ERR2517164, ERR2517422, ERR2517353, ERR2517516, ERR2517478, ERR2517404, ERR2517293, ERR2517405, ERR2517495, ERR2517255, ERR2517361, ERR2517189, ERR2517510, ERR2517301, ERR2517652, ERR2517352, ERR2517610, ERR2517211, ERR2517338, ERR2517461, ERR2517611, ERR2517425, ERR2517302, ERR2517295, ERR2517512, ERR2517256, ERR2517386, ERR2517582, ERR2517542, ERR2517397, ERR2517223, ERR2517263, ERR2517482, ERR2517452, ERR2517363, ERR2517595, ERR2517129, ERR2517197, ERR2517503, ERR2517281, ERR2517531, ERR2517507, ERR2517477, ERR2517242, ERR2517236, ERR2517143, ERR2517306, ERR2517538, ERR2517271, ERR2517450, ERR2517326, ERR2517303, ERR2517431, ERR2517517, ERR2517513, ERR2517469, ERR2517259, ERR2517499, ERR2517614, ERR2517137, ERR2517537, ERR2517419, ERR2517279, ERR2517641, ERR2517355, ERR2517556, ERR2517380, ERR2517185, ERR2517648, ERR2517163, ERR2517619, ERR2517650, ERR2517468, ERR2517175, ERR2517617, ERR2517488, ERR2517549, ERR2517630, ERR2517340, ERR2517196, ERR2517472, ERR2517395, ERR2517634, ERR2517162, ERR2517483, ERR2517546, ERR2517241, ERR2517131, ERR2517449, ERR2517222, ERR2517261, ERR2517480, ERR2517441, ERR2517536, ERR2517382, ERR2517316, ERR2517526, ERR2517505, ERR2517616, ERR2517585, ERR2517381, ERR2517631, ERR2517210, ERR2517518, ERR2517473, ERR2517233, ERR2517554, ERR2517557, ERR2517264, ERR2517486, ERR2517502, ERR2517270, ERR2517246, ERR2517274, ERR2517311, ERR2517448, ERR2517522, ERR2517583, ERR2517442, ERR2517324, ERR2517640, ERR2517543, ERR2517372, ERR2517587, ERR2517457, ERR2517337, ERR2517451, ERR2517378, ERR2517283, ERR2517586, ERR2517168, ERR2517376, ERR2517403, ERR2517566, ERR2517272, ERR2517476, ERR2517345, ERR2517273, ERR2517592, ERR2517424, ERR2517622, ERR2517252, ERR2517331, ERR2517322, ERR2517445, ERR2517138, ERR2517447, ERR2517248, ERR2517180, ERR2517400, ERR2517282, ERR2517565, ERR2517328, ERR2517561, ERR2517379, ERR2517198, ERR2517285, ERR2517366, ERR2517573, ERR2517389, ERR2517637, ERR2517144, ERR2517576, ERR2517541, ERR2517626, ERR2517481, ERR2517535, ERR2517357, ERR2517629, ERR2517343, ERR2517369, ERR2517500, ERR2517327, ERR2517597, ERR2517387, ERR2517413, ERR2517607, ERR2517158, ERR2517394, ERR2517149, ERR2517396, ERR2517581, ERR2517653, ERR2517169, ERR2517312, ERR2517459, ERR2517288, ERR2517128, ERR2517173, ERR2517438, ERR2517574, ERR2517141, ERR2517318, ERR2517479, ERR2517190, ERR2517347, ERR2517313, ERR2517181, ERR2517570, ERR2517520, ERR2517527, ERR2517257, ERR2517251, ERR2517443, ERR2517562, ERR2517454, ERR2517309, ERR2517571, ERR2517642, ERR2517651, ERR2517152, ERR2517625, ERR2517429, ERR2517644, ERR2517368, ERR2517305, ERR2517414, ERR2517160, ERR2517635, ERR2517645, ERR2517511, ERR2517321, ERR2517291, ERR2517603, ERR2517606, ERR2517349, ERR2517364, ERR2517421, ERR2517388, ERR2517250, ERR2517446, ERR2517509, ERR2517151, ERR2517289, ERR2517290, ERR2517496, ERR2517207, ERR2517299, ERR2517636, ERR2517200, ERR2517280, ERR2517176, ERR2517620, ERR2517474, ERR2517466, ERR2517467, ERR2517613, ERR2517415, ERR2517401, ERR2517237, ERR2517224, ERR2517286, ERR2517249, ERR2517416, ERR2517453, ERR2517212, ERR2517146, ERR2517490, ERR2517533, ERR2517247, ERR2517609, ERR2517359, ERR2517601, ERR2517304, ERR2517240, ERR2517195, ERR2517612, ERR2517418, ERR2517214, ERR2517471, ERR2517367, ERR2517231, ERR2517639, ERR2517209, ERR2517475, ERR2517528, ERR2517216, ERR2517325, ERR2517621, ERR2517628, ERR2517307, ERR2517135, ERR2517348, ERR2517420, ERR2517230, ERR2517530, ERR2517439, ERR2517506, ERR2517132, ERR2517437, ERR2517432, ERR2517365, ERR2517296, ERR2517208, ERR2517623, ERR2517172, ERR2517426, ERR2517489, ERR2517588, ERR2517493, ERR2517157, ERR2517351, ERR2517206, ERR2517267, ERR2517456, ERR2517213, ERR2517584, ERR2517463, ERR2517539, ERR2517550, ERR2517314, ERR2517646, ERR2517339, ERR2517523, ERR2517317, ERR2517292, ERR2517294, ERR2517170, ERR2517238, ERR2517350, ERR2517579, ERR2517139, ERR2517156, ERR2517430, ERR2517407, ERR2517519, ERR2517417, ERR2517342, ERR2517558, ERR2517605, ERR2517370, ERR2517532, ERR2517375, ERR2517534, ERR2517187, ERR2517392, ERR2517134, ERR2517362, ERR2517508, ERR2517218, ERR2517555, ERR2517647, ERR2517150, ERR2517529, ERR2517551, ERR2517377, ERR2517225, ERR2517545, ERR2517232, ERR2517423, ERR2517491, ERR2517462, ERR2517575, ERR2517553, ERR2517525, ERR2517166, ERR2517514, ERR2517145, ERR2517524, ERR2517332, ERR2517142, ERR2517277, ERR2517540, ERR2517171, ERR2517186, ERR2517228, ERR2517409, ERR2517504, ERR2517133, ERR2517126, ERR2517234, ERR2517329, ERR2517638, ERR2517227, ERR2517544, ERR2517136, ERR2517393, ERR2517402, ERR2517406, ERR2517633, ERR2517521, ERR2517434, ERR2517360, ERR2509673, ERR2509880, ERR2509885, ERR2509674, ERR2509682, ERR2509891, ERR2509685, ERR2509879, ERR2509895, ERR2509675, ERR2509676, ERR2509882, ERR2509684, ERR2509677, ERR2509888, ERR2509890, ERR2509881, ERR2509894, ERR2509886, ERR2509892, ERR2509678, ERR2509679, ERR2509681, ERR2509896, ERR2509680, ERR2509897, ERR2509686, ERR2509889, ERR2509683, ERR2509887, ERR2509893, ERR2509883, SRR6367395, SRR6339663, ERR2510632, ERR2510285, ERR2510586, ERR2510385, ERR2510610, ERR2510370, ERR2510448, ERR2510628, ERR2510563, ERR2510248, ERR2510592, ERR2510597, ERR2510294, ERR2510452, ERR2510576, ERR2510402, ERR2510585, ERR2510630, ERR2510526, ERR2510587, ERR2510429, ERR2510498, ERR2510558, ERR2510524, ERR2510601, ERR2510296, ERR2510251, ERR2510651, ERR2510540, ERR2510431, ERR2510619, ERR2510600, ERR2510319, ERR2510307, ERR2510528, ERR2510527, ERR2510532, ERR2510648, ERR2510599, ERR2510442, ERR2510639, ERR2510871, ERR2510593, ERR2510263, ERR2510644, ERR2510343, ERR2510256, ERR2510514, ERR2510292, ERR2510378, ERR2510443, ERR2510276, ERR2510571, ERR2510545, ERR2510391, ERR2510578, ERR2510453, ERR2510566, ERR2510464, ERR2510299, ERR2510647, ERR2510549, ERR2510546, ERR2510483, ERR2510388, ERR2510650, ERR2510463, ERR2510572, ERR2510573, ERR2510390, ERR2510598, ERR2510485, ERR2510535, ERR2510326, ERR2510458, ERR2510624, ERR2510346, ERR2510435, ERR2510530, ERR2510564, ERR2510428, ERR2510405, ERR2510581, ERR2510474, ERR2510555, ERR2510322, ERR2510304, ERR2510271, ERR2510421, ERR2510638, ERR2510342, ERR2510344, ERR2510634, ERR2510328, ERR2510399, ERR2510281, ERR2510414, ERR2510861, ERR2510473, ERR2510583, ERR2510400, ERR2510559, ERR2510605, ERR2510461, ERR2510589, ERR2510479, ERR2510436, ERR2510353, ERR2510565, ERR2510471, ERR2510604, ERR2510330, ERR2510477, ERR2510262, ERR2510879, ERR2510310, ERR2510286, ERR2510287, ERR2510557, ERR2510548, ERR2510499, ERR2510455, ERR2510472, ERR2510469, ERR2510250, ERR2510506, ERR2510249, ERR2510419, ERR2510529, ERR2510395, ERR2510625, ERR2510412, ERR2510393, ERR2510538, ERR2510403, ERR2510387, ERR2510494, ERR2510380, ERR2510534, ERR2510450, ERR2510397, ERR2510873, ERR2510612, ERR2510337, ERR2510627, ERR2510255, ERR2510257, ERR2510406, ERR2510357, ERR2510492, ERR2510301, ERR2510386, ERR2510641, ERR2510369, ERR2510317, ERR2510552, ERR2510308, ERR2510636, ERR2510398, ERR2510341, ERR2510384, ERR2510445, ERR2510340, ERR2510290, ERR2510364, ERR2510594, ERR2510481, ERR2510561, ERR2510574, ERR2510265, ERR2510622, ERR2510275, ERR2510313, ERR2510603, ERR2510577, ERR2510613, ERR2510512, ERR2510523, ERR2510273, ERR2510277, ERR2510590, ERR2510389, ERR2510244, ERR2510306, ERR2510580, ERR2510413, ERR2510507, ERR2510352, ERR2510427, ERR2510635, ERR2510480, ERR2510513, ERR2510367, ERR2510332, ERR2510646, ERR2510596, ERR2510615, ERR2510316, ERR2510269, ERR2510366, ERR2510350, ERR2510468, ERR2510323, ERR2510289, ERR2510457, ERR2510318, ERR2510423, ERR2510300, ERR2510460, ERR2510408, ERR2510482, ERR2510470, ERR2510416, ERR2510336, ERR2510588, ERR2510373, ERR2510495, ERR2510278, ERR2510434, ERR2510584, ERR2510243, ERR2510440, ERR2510293, ERR2510522, ERR2510553, ERR2510501, ERR2510462, ERR2510437, ERR2510396, ERR2510519, ERR2510502, ERR2510487, ERR2510537, ERR2510447, ERR2510567, ERR2510267, ERR2510348, ERR2510320, ERR2510525, ERR2510493, ERR2510476, ERR2510616, ERR2510407, ERR2510614, ERR2510521, ERR2510520, ERR2510883, ERR2510358, ERR2510259, ERR2510359, ERR2510697, ERR2510631, ERR2510409, ERR2510325, ERR2510295, ERR2510497, ERR2510404, ERR2510602, ERR2510371, ERR2510441, ERR2510608, ERR2510266, ERR2510303, ERR2510246, ERR2510354, ERR2510484, ERR2510274, ERR2510467, ERR2510575, ERR2510418, ERR2510611, ERR2510382, ERR2510375, ERR2510424, ERR2510870, ERR2510568, ERR2510430, ERR2510486, ERR2510554, ERR2510376, ERR2510363, ERR2510475, ERR2510626, ERR2510505, ERR2510439, ERR2510311, ERR2510377, ERR2510252, ERR2510542, ERR2510253, ERR2510415, ERR2510860, ERR2510633, ERR2510381, ERR2510422, ERR2510543, ERR2510333, ERR2510280, ERR2510508, ERR2510410, ERR2510547, ERR2510570, ERR2510338, ERR2510569, ERR2510698, ERR2510649, ERR2510392, ERR2510515, ERR2510582, ERR2510444, ERR2510478, ERR2510451, ERR2510446, ERR2510517, ERR2510606, ERR2510309, ERR2510643, ERR2510544, ERR2510518, ERR2510315, ERR2510335, ERR2510362, ERR2510640, ERR2510425, ERR2510541, ERR2510305, ERR2510489, ERR2510539, ERR2510579, ERR2510516, ERR2510503, ERR2510438, ERR2510466, ERR2510284, ERR2510411, ERR2510302, ERR2510511, ERR2510291, ERR2510368, ERR2510394, ERR2510591, ERR2510298, ERR2510621, ERR2510500, ERR2510339, ERR2510652, ERR2510258, ERR2510550, ERR2510562, ERR2510556, ERR2510433, ERR2510417, ERR2510401, ERR2510533, ERR2510426, ERR2510351, ERR2510490, ERR2510379, ERR2510372, ERR2510618, ERR2510536, ERR2510620, ERR2510334, ERR2510617, ERR2510347, ERR2510365, ERR2510312, ERR2510531, ERR2510449, ERR2510245, ERR2510504, ERR2510645, ERR2510560, ERR2510551, ERR2510324, ERR2510595, ERR2510356, ERR2510465, ERR2510491, ERR2510361, ERR2510349, ERR2510261, ERR2510297, ERR2510260, ERR2510609, ERR2510509, ERR2510355, ERR2510282, ERR2510623, ERR2510321, ERR2510272, ERR2510629, ERR2510607, ERR2510488, ERR2041785, ERR2041771, ERR2200148, ERR2041688, ERR2041773, ERR2041780, ERR2041741, ERR2041696, ERR2041734, ERR2041709, ERR2041786, ERR2041746, ERR2041727, ERR2041788, ERR2041753, ERR2041783, ERR2041782, ERR2041758, ERR2041744, ERR2041742, ERR2041706, ERR2041794, ERR2041793, ERR2041683, ERR2041721, ERR2041760, ERR2041767, ERR2041689, ERR2041770, ERR2041756, ERR2041804, ERR2041764, ERR2041752, ERR2041723, ERR2041805, ERR2041761, ERR2041803, ERR2200092, ERR2041684, ERR2041714, ERR2041732, ERR2041733, ERR2041766, ERR2041777, ERR2041775, ERR2041708, ERR2041718, ERR2041736, ERR2041762, ERR2041685, ERR2041776, ERR2041757, ERR2041795, ERR2041730, ERR2041747, ERR2041719, ERR2041774, ERR2041739, ERR2041789, ERR2041729, ERR2041716, ERR2041749, ERR2041702, ERR2041700, ERR2041738, ERR2041765, ERR2041745, ERR2041735, ERR2199914, ERR2041693, ERR2041769, ERR2041800, ERR2041711, ERR2041731, ERR2041791, ERR2041768, ERR2041750, ERR2041772, ERR2041759, ERR2041798, ERR2041751, ERR2041787, ERR2041802, ERR2041712, ERR2041704, ERR2041717, ERR2200070, ERR2041720, ERR2041781, ERR2041796, ERR2041682, ERR2200093, ERR2041722, ERR2041698, ERR2041754, ERR2041784, ERR2041763, ERR2041743, ERR2041748, ERR2041724, ERR2041728, ERR2041755, ERR2515105, ERR2515106, ERR2515107, ERR2515108, ERR2515109, ERR2515110, ERR2515111, ERR2515112, ERR2515113, ERR2515114, ERR2515116, ERR2515117, ERR2515118, ERR2515119, ERR2515120, ERR2515121, ERR2515122, ERR2515123, ERR2515124, ERR2515125, ERR2515126, ERR2515127, ERR2515128, ERR2515129, ERR2515130, ERR2515131, ERR2515132, ERR2515134, ERR2515136, ERR2515137, ERR2515138, ERR2515139, ERR2515140, ERR2515141, ERR2515143, ERR2515145, ERR2515146, ERR2515147, ERR2515148, ERR2515149, ERR2515150, ERR2515151, ERR2515154, ERR2515155, ERR2515156, ERR2515157, ERR2515158, ERR2515160, ERR2515161, ERR2515162, ERR2515163, ERR2515164, ERR2515166, ERR2515167, ERR2515168, ERR2515169, ERR2515170, ERR2515171, ERR2515172, ERR2515174, ERR2515176, ERR2515177, ERR2515178, ERR2515179, ERR2515180, ERR2515181, ERR2515182, ERR2515183, ERR2515184, ERR2515185, ERR2515186, ERR2515187, ERR2515188, ERR2515189, ERR2515190, ERR2515192, ERR2515193, ERR2515194, ERR2515195, ERR2515196, ERR2515197, ERR2515198, ERR2515200, ERR2515201, ERR2515202, ERR2515203, ERR2515204, ERR2515205, ERR2515206, ERR2515207, ERR2515208, ERR2515209, ERR2515210, ERR2515211, ERR2515212, ERR2515214, ERR2515215, ERR2515216, ERR2515217, ERR2515218, ERR2515219, ERR2515220, ERR2515221, ERR2515222, ERR2515224, ERR2515225, ERR2515226, ERR2515227, ERR2515228, ERR2515229, ERR2515230, ERR2515231, ERR2515232, ERR2515233, ERR2515234, ERR2515235, ERR2515236, ERR2515237, ERR2515239, ERR2515240, ERR2515241, ERR2515242, ERR2515243, ERR2515244, ERR2515245, ERR2515246, ERR2515247, ERR2515249, ERR2515251, ERR2515252, ERR2515253, ERR2515254, ERR2515255, ERR2515256, ERR2515257, ERR2515258, ERR2515259, ERR2515260, ERR2515261, ERR2515262, ERR2515264, ERR2515265, ERR2515266, ERR2515267, ERR2515268, ERR2515269, ERR2515270, ERR2515272, ERR2515273, ERR2515274, ERR2515275, ERR2515276, ERR2515277, ERR2515278, ERR2515279, ERR2515280, ERR2515281, ERR2515282, ERR2515283, ERR2515284, ERR2515285, ERR2515287, ERR2515288, ERR2515289, ERR2515290, ERR2515291, ERR2515292, ERR2515293, ERR2515294, ERR2515295, ERR2515297, ERR2515298, ERR2515299, ERR2515300, ERR2515301, ERR2515302, ERR2515303, ERR2515304, ERR2515305, ERR2515306, ERR2515307, ERR2515308, ERR2515309, ERR2515310, ERR2515311, ERR2515312, ERR2515313, ERR2515314, ERR2515315, ERR2515316, ERR2515317, ERR2515318, ERR2515320, ERR2515321, ERR2515323, ERR2515324, ERR2515326, ERR2515327, ERR2515328, ERR2515329, ERR2515330, ERR2515332, ERR2515334, ERR2515336, ERR2515338, ERR2515339, ERR2515340, ERR2515341, ERR2515342, ERR2515343, ERR2515345, ERR2515346, ERR2515347, ERR2515348, ERR2515349, ERR2515350, ERR2515351, ERR2515352, ERR2515353, ERR2515355, ERR2515356, ERR2515357, ERR2515359, ERR2515360, ERR2515362, ERR2515363, ERR2515364, ERR2515365, ERR2515366, ERR2515367, ERR2515368, ERR2515369, ERR2515370, ERR2515371, ERR2515372, ERR2515374, ERR2515375, ERR2515376, ERR2515377, ERR2515378, ERR2515379, ERR2515380, ERR2515381, ERR2515382, ERR2515383, ERR2515384, ERR2515385, ERR2515387, ERR2515388, ERR2515389, ERR2515390, ERR2515391, ERR2515392, ERR2515393, ERR2515394, ERR2515395, ERR2515396, ERR2515397, ERR2515398, ERR2515399, ERR2515400, ERR2515401, ERR2515402, ERR2515403, ERR2515404, ERR2515406, ERR2515407, ERR2515408, ERR2515409, ERR2515410, ERR2515412, ERR2515413, ERR2515414, ERR2515416, ERR2515417, ERR2515418, ERR2515420, ERR2515421, ERR2515422, ERR2515423, ERR2515424, ERR2515426, ERR2515428, ERR2515429, ERR2515431, ERR2515432, ERR2515433, ERR2515434, ERR2515435, ERR2515436, ERR2515437, ERR2515438, ERR2515439, ERR2515440, ERR2515441, ERR2515442, ERR2515443, ERR2515444, ERR2515445, ERR2515446, ERR2515447, ERR2515448, ERR2515449, ERR2515450, ERR2515451, ERR2515452, ERR2515453, ERR2515454, ERR2515455, ERR2515456, ERR2515457, ERR2515459, ERR2515460, ERR2515461, ERR2515462, ERR2515463, ERR2515464, ERR2515465, ERR2515466, ERR2515467, ERR2515468, ERR2515469, ERR2515470, ERR2515471, ERR2515472, ERR2515474, ERR2515476, ERR2515477, ERR2515478, ERR2515480, ERR2515481, ERR2515482, ERR2515483, ERR2515484, ERR2515485, ERR2515486, ERR2515487, ERR2515488, ERR2515489, ERR2515490, ERR2515491, ERR2515492, ERR2515493, ERR2515494, ERR2515495, ERR2515496, ERR2515497, ERR2515498, ERR2515499, ERR2515500, ERR2515501, ERR2515502, ERR2515503, ERR2515504, ERR2515505, ERR2515506, ERR2515515, ERR2515516, ERR2515517, ERR2515518, ERR2515521, ERR2515522, ERR2515523, ERR2515524, ERR2515525, ERR2515526, ERR2515527, ERR2515529, ERR2515530, ERR2515531, ERR2515532, ERR2515533, ERR2515534, ERR2515535, ERR2515536, ERR2515537, ERR2515538, ERR2515539, ERR2515540, ERR2515542, ERR2515543, ERR2515545, ERR2515547, ERR2515548, ERR2515549, ERR2515550, ERR2515551, ERR2515552, ERR2515553, ERR2515554, ERR2515555, ERR2515556, ERR2515557, ERR2515558, ERR2515559, ERR2515561, ERR2515562, ERR2515563, ERR2515564, ERR2515565, ERR2515566, ERR2515567, ERR2515569, ERR2515570, ERR2515571, ERR2515572, ERR2515573, ERR2515574, ERR2515576, ERR2515577, ERR2515578, ERR2515579, ERR2515580, ERR2515581, ERR2515582, ERR2515585, ERR2515586, ERR2515587, ERR2515592, ERR2515593, ERR2515595, ERR2515596, ERR2515598, ERR2515599, ERR2515600, ERR2515601, ERR2515602, ERR2515603, ERR2515604, ERR2515605, ERR2515606, ERR2515607, ERR2515608, ERR2515610, ERR2515611, ERR2515612, ERR2515613, ERR2515614, ERR2515615, ERR2515616, ERR2515617, ERR2515618, ERR2515619, ERR2515620, ERR2515622, ERR2515623, ERR2515624, ERR2515625, ERR2515626, ERR2515627, ERR2515628, ERR2515629, ERR2515630, ERR2515631, ERR2515632, ERR2515633, ERR2515634, ERR2515635, ERR2515637, ERR2515638, ERR2515639, ERR2515642, ERR2515643, ERR2515644, ERR2515645, ERR2515646, ERR2515648, ERR2515649, ERR2515651, ERR2515652, ERR2515653, ERR2515654, ERR2515655, ERR2515656, ERR2515657, ERR2515659, ERR2515661, ERR2515662, ERR2515663, ERR2515664, ERR2515665, ERR2515666, ERR2515668, ERR2515670, ERR2515671, ERR2515672, ERR2515673, ERR2515674, ERR2515675, ERR2515676, ERR2515677, ERR2515678, ERR2515679, ERR2515680, ERR2515681, ERR2515682, ERR2515683, ERR2515684, ERR2515685, ERR2515686, ERR2515687, ERR2515688, ERR2515689, ERR2515690, ERR2515691, ERR2515692, ERR2515693, ERR2515694, ERR2515695, ERR2515696, ERR2515697, ERR2515698, ERR2515699, ERR2515700, ERR2515701, ERR2515702, ERR2515703, ERR2515705, ERR2515706, ERR2515707, ERR2515709, ERR2515711, ERR2515712, ERR2515713, ERR2515714, ERR2515715, ERR2515716, ERR2515717, ERR2515718, ERR2515720, ERR2515721, ERR2515722, ERR2515723, ERR2515724, ERR2515726, ERR2515727, ERR2515728, ERR2515729, ERR2515730, ERR2515731, ERR2515733, ERR2515734, ERR2515735, ERR2515736, ERR2515737, ERR2515738, ERR2515740, ERR2515741, ERR2515742, ERR2515744, ERR2515745, ERR2515748, ERR2515749, ERR2515750, ERR2515751, ERR2515752, ERR2515753, ERR2515755, ERR2515757, ERR2515758, ERR2515759, ERR2515760, ERR2515761, ERR2515762, ERR2515763, ERR2515764, ERR2515765, ERR2515766, ERR2515767, ERR2515769, ERR2515770, ERR2515771, ERR2515772, ERR2515773, ERR2515774, ERR2515775, ERR2515777, ERR2515778, ERR2515779, ERR2515780, ERR2515781, ERR2515782, ERR2515783, ERR2515784, ERR2515785, ERR2515786, ERR2515787, ERR2515788, ERR2515790, ERR2515791, ERR2515792, ERR2515793, ERR2515795, ERR2515796, ERR2515798, ERR2515799, ERR2515800, ERR2515801, ERR2515803, ERR2515804, ERR2515805, ERR2515807, ERR2515808, ERR2515809, ERR2515811, ERR2515813, ERR2515814, ERR2515815, ERR2515816, ERR2515817, ERR2515818, ERR2515819, ERR2515820, ERR2515821, ERR2515822, ERR2515823, ERR2515824, ERR2515825, ERR2515827, ERR2515828, ERR2515829, ERR2515830, ERR2515831, ERR2515832, ERR2515836, ERR2515838, ERR2515840, ERR2515841, ERR2515842, ERR2515843, ERR2515844, ERR2515845, ERR2515846, ERR2515847, ERR2515848, ERR2515849, ERR2515850, ERR2515851, ERR2515852, ERR2515853, ERR2515854, ERR2515855, ERR2515856, ERR2515857, ERR2515858, ERR2515859, ERR2515861, ERR2515862, ERR2515863, ERR2515865, ERR2515866, ERR2515868, ERR2515869, ERR2515870, ERR2515871, ERR2515873, ERR2515874, ERR2515875, ERR2515876, ERR2515877, ERR2515879, ERR2515880, ERR2515881, ERR2515882, ERR2515883, ERR2515884, ERR2515885, ERR2515886, ERR2515887, ERR2515888, ERR2515889, ERR2515890, ERR2515891, ERR2515892, ERR2515893, ERR2515894, ERR2515895, ERR2515896, ERR2515897, ERR2515899, ERR2515900, ERR2515901, ERR2515902, ERR2515903, ERR2515904, ERR2515905, ERR2515906, ERR2515907, ERR2515908, ERR2515909, ERR2515912, ERR2515913, ERR2515914, ERR2515915, ERR2515916, ERR2515917, ERR2515919, ERR2515921, ERR2515922, ERR2515923, ERR2515925, ERR2515927, ERR2515928, ERR2515929, ERR2515930, ERR2515932, ERR2515933, ERR2515934, ERR2515935, ERR2515936, ERR2515937, ERR2515938, ERR2515939, ERR2515940, ERR2515941, ERR2515942, ERR2515944, ERR2515945, ERR2515946, ERR2515947, ERR2515948, ERR2515949, ERR2515950, ERR2515951, ERR2515952, ERR2515953, ERR2515954, ERR2515955, ERR2515956, ERR2515957, ERR2515958, ERR2515959, ERR2515960, ERR2515961, ERR2515962, ERR2515963, ERR2515964, ERR2515966, ERR2515967, ERR2515968, ERR2515969, ERR2515970, ERR2515972, ERR2515973, ERR2515974, ERR2515975, ERR2515976, ERR2515977, ERR2515978, ERR2515979, ERR2515980, ERR2515981, ERR2515982, ERR2515983, ERR2515984, ERR2515985, ERR2515986, ERR2515987, ERR2515989, ERR2515991, ERR2515992, ERR2515993, ERR2515994, ERR2515995, ERR2515997, ERR2515998, ERR2515999, ERR2516000, ERR2516001, ERR2516002, ERR2516003, ERR2516004, ERR2516006, ERR2516007, ERR2516008, ERR2516009, ERR2516010, ERR2516011, ERR2516013, ERR2516015, ERR2516016, ERR2516020, ERR2516021, ERR2516022, ERR2516024, ERR2516025, ERR2516026, ERR2516027, ERR2516028, ERR2516029, ERR2516030, ERR2516031, ERR2516032, ERR2516033, ERR2516034, ERR2516035, ERR2516036, ERR2516037, ERR2516038, ERR2516039, ERR2516040, ERR2516042, ERR2516043, ERR2516044, ERR2516046, ERR2516047, ERR2516048, ERR2516050, ERR2516051, ERR2516052, ERR2516053, ERR2516054, ERR2516056, ERR2516057, ERR2516058, ERR2516060, ERR2516061, ERR2516062, ERR2516065, ERR2516067, ERR2516069, ERR2516070, ERR2516071, ERR2516072, ERR2516073, ERR2516074, ERR2516075, ERR2516076, ERR2516078, ERR2516080, ERR2516081, ERR2516082, ERR2516083, ERR2516084, ERR2516085, ERR2516086, ERR2516087, ERR2516088, ERR2516089, ERR2516090, ERR2516091, ERR2516092, ERR2516093, ERR2516094, ERR2516095, ERR2516096, ERR2516097, ERR2516098, ERR2516099, ERR2516100, ERR2516101, ERR2516102, ERR2516103, ERR2516104, ERR2516105, ERR2516106, ERR2516107, ERR2516108, ERR2516109, ERR2516110, ERR2516111, ERR2516112, ERR2516113, ERR2516114, ERR2516115, ERR2516116, ERR2516117, ERR2516118, ERR2516119, ERR2516121, ERR2516122, ERR2516123, ERR2516124, ERR2516125, ERR2516126, ERR2516127, ERR2516128, ERR2516129, ERR2516130, ERR2516131, ERR2516132, ERR2516133, ERR2516134, ERR2516135, ERR2516136, ERR2516137, ERR2516138, ERR2516139, ERR2516140, ERR2516142, ERR2516143, ERR2516144, ERR2516145, ERR2516147, ERR2516148, ERR2516149, ERR2516150, ERR2516151, ERR2516152, ERR2516153, ERR2516154, ERR2516155, ERR2516156, ERR2516157, ERR2516158, ERR2516159, ERR2516160, ERR2516161, ERR2516162, ERR2516163, ERR2516164, ERR2516165, SRR6369878, SRR6369877, SRR6369876, SRR6369875, SRR6367401, SRR6367400, SRR6367399, SRR6367398, SRR6367396, SRR6339667, SRR6339666, SRR6339665, SRR6339664, SRR6339662, SRR6339661, SRR6339653, SRR6339651, SRR6339649, SRR6339648, SRR6339645, SRR6339644, SRR6339643, SRR6339642, SRR6339641, SRR6339640, SRR6339637, SRR5817481, SRR5817480, SRR5817479, SRR5817478, SRR5817475, SRR5817473, SRR5817472, SRR5817471, SRR5817470, SRR5817469, SRR5817467, SRR5817466, SRR5817464, SRR5817463, SRR2333215, SRR2328057

TABLE A
Isoniazid weighted positions; threshold value: -1.166

| POS | RN | SN | W | POS | RN | SN | W | POS | RN | SN | W |
|---|---|---|---|---|---|---|---|---|---|---|---|
| 107937 | ACC | ACCC | -1.611 | 2155541 | A | G | -0.4418 | 4327448 | A | C | -2.066 |
| 1083962 | C | A | -3.895 | 2155546 | T | C | -1.924 | 4328019 | C | T | -0.632 |
| 1137943 | C | T | -1.732 | 2155546 | T | G | -1.232 | 4328037 | G | A | -2.47 |
| 1237557 | A | C | -1.611 | 2155563 | G | T | -0.6722 | 4328059 | C | A | -3.895 |
| 1237609 | C | A | -3.895 | 2155607 | C | T | -2.233 | 4399252 | AGC | GGG | -0.599 |
| 1416409 | C | G | -3.347 | 2155624 | T | G | -3.624 | 4400008 | G | A | -3.895 |
| 1416432 | C | T | -3.236 | 2155636 | A | G | -3.576 | 4403518 | T | C | -0.1727 |
| 1417140 | C | T | -0.018 | 2155648 | T | C | -2.761 | 4407583 | C | T | -2.591 |
| 1472041 | C | T | -4.128 | 2155687 | T | C | -0.4141 | 4407731 | GCC | GC | -1.326 |
| 1472069 | G | T | -1.611 | 2155689 | C | G | -2.727 | 4407793 | C | T | -4.128 |
| 1472127 | CGG | TGCTCT | -3.895 | 2155738 | C | T | -3.784 | 4407824 | G | A | -3.895 |
| 1472359 | A | C | -0.223 | 2155740 | GCCCCC | GCCCC | -2.319 | 4407846 | GGCGC | GGCGC | -3.895 |
| | | | | | G | G | | | GCC | C | |
| 1472362 | C | T | -0.046 | 2155786 | G | A | -3.624 | 4407896 | C | A | -0.08956 |
| 1472428 | G | T | -3.443 | 2155787 | C | T | -2.382 | 4407913 | C | T | -0.7143 |
| 1472487 | CTA | CTTA | -4.154 | 2155801 | C | T | -2.062 | 4407927 | T | G | -0.06442 |
| 1472753 | A | T | -3.502 | 2155817 | C | T | -2.81 | 4407976 | C | T | -1.889 |
| 1472772 | C | T | -3.895 | 2155844 | A | G | -2.327 | 4407979 | G | C | -0.7918 |
| 1472866 | C | T | -3.826 | 2155959 | GTT | GT | -0.1331 | 4407985 | C | G | -0.4042 |
| 1473246 | A | G | -0.881 | 2156066 | C | G | -3.856 | 4407994 | C | T | -3.56 |
| 1473254 | A | C | -0.745 | 2223293 | T | C | 3.263 | 4408006 | A | G | -3.895 |
| 1473260 | G | T | -2.098 | 2223757 | G | A | -1.037 | 4408066 | TCC | TC | -3.895 |
| 1473959 | TTGT | GTGG | -1.233 | 2279395 | C | T | -1.982 | 4408100 | GCCCCC | GCCCC | -0.7235 |
| | | | | | | | | | G | G | |
| 1474313 | T | C | -3.895 | 2279868 | GCCT | GCCCCT | -3.895 | 4408102 | C | A | 0.7047 |
| 1474686 | A | G | -2.403 | 2288730 | G | T | 3.732 | 4408159 | C | G | -1.839 |
| 1475116 | G | T | -0.218 | 2288932 | TGTACG | TG | -2.647 | 496582 | C | T | -3.895 |
| 1475751 | C | T | -3.850 | 2289214 | G | A | -1.81 | 497248 | T | G | -0.7344 |
| 1476442 | T | C | -4.387 | 2412005 | A | C | -0.7962 | 510741 | C | A | -2.349 |
| 1490779 | C | T | -4.128 | 2518871 | C | T | -3.78 | 511087 | A | C | -3.253 |
| 1491719 | C | T | -0.767 | 2694045 | C | G | -0.1311 | 511319 | G | T | 1.864 |
| 1501288 | C | T | -3.715 | 2694909 | G | A | 0.6835 | 513282 | G | A | -1.611 |
| 1501977 | A | C | -0.644 | 2714465 | G | T | -1.611 | 514180 | C | G | -1.338 |
| 157174 | T | C | -0.251 | 2714929 | G | T | -1.035 | 514876 | G | C | 0.3029 |
| 1673425 | C | T | -2.339 | 2715215 | C | T | -2.332 | 515110 | T | G | 0.2691 |
| 1674048 | G | A | -0.183 | 2726136 | C | T | 1.164 | 538698 | A | C | -3.895 |
| 1674248 | T | C | -1.759 | 2726146 | GA | GTA | 0.2011 | 540078 | C | G | -2.858 |
| 1674262 | A | G | -2.415 | 2726480 | G | T | -4.128 | 5413 | G | A | -0.02688 |
| 1674263 | T | C | -3.551 | 2726775 | G | A | -3.895 | 5416 | C | T | -1.902 |
| 1674481 | T | G | -0.950 | 273788 | G | T | -2.167 | 5440 | A | C | 2.137 |
| 1674769 | G | T | -2.135 | 2751831 | G | T | -4.128 | 5446 | G | T | -1.765 |
| 1674772 | G | A | -2.030 | 2751934 | GGA | GGC | -2.519 | 5548 | A | G | -1.823 |
| 1674782 | T | C | -2.877 | 2752122 | G | C | -0.04813 | 5548 | A | C | -1.759 |
| 1718818 | G | C | -3.895 | 2937452 | C | T | -3.895 | 5597 | C | T | -4.128 |
| 1758093 | C | G | -0.248 | 2939373 | G | C | 0.2417 | 575544 | G | T | 0.5595 |
| 1833555 | C | G | -1.869 | 2939510 | C | G | -0.9458 | 575608 | G | A | 2.37 |
| 1833594 | C | A | 0.111 | 2939609 | C | A | -3.895 | 575797 | C | T | -3.994 |
| 1833906 | A | T | -3.912 | 2939758 | C | T | -3.895 | 576316 | A | G | -2.08 |
| 1834177 | A | C | -0.103 | 2940759 | CCCG | CCCC | 0.2827 | 576472 | C | A | -2.287 |
| 1834279 | C | T | -3.994 | 2955150 | G | A | -0.2147 | 576526 | C | T | -2.935 |
| 1834540 | G | T | -0.320 | 2956140 | A | G | -0.8646 | 576660 | T | G | -1.611 |
| 1849536 | C | T | -3.895 | 2956571 | G | C | -1.611 | 576693 | A | C | -0.609 |
| 1850296 | G | A | -1.228 | 3074361 | A | G | -3.895 | 576751 | G | C | -0.1557 |
| 1851131 | T | G | -1.469 | 3272239 | T | G | -0.1907 | 5849 | C | A | -1.528 |
| 1851384 | C | A | 0.101 | 3296300 | C | A | 1.352 | 5980 | G | C | -4.128 |
| 1875470 | T | G | -3.624 | 3296919 | T | C | -0.1295 | 6016 | C | G | -0.8755 |
| 1875502 | GTT | GT | -3.376 | 3296962 | G | C | -3.624 | 61178 | C | T | -3.895 |
| 1875567 | G | C | -2.737 | 3296992 | G | C | -2.22 | 614975 | G | C | -0.1391 |
| 1875600 | C | T | -0.498 | 3297120 | CGG | CG | 3.406 | 61988 | C | T | -3.895 |
| 1875709 | T | G | -0.539 | 3297442 | CAT | CAAT | 0.7593 | 62196 | G | A | -1.551 |
| 1875902 | T | C | -0.900 | 3326394 | A | G | -3.895 | 6302 | G | T | -2.822 |
| 1876608 | C | G | -0.745 | 3388356 | A | C | -3.754 | 6358 | C | T | 0.3993 |
| 1877312 | G | T | -3.290 | 3465908 | G | C | -1.077 | 6794 | A | G | -3.994 |
| 1877638 | A | G | -0.799 | 347401 | C | G | -3.895 | 7170 | C | A | -2.705 |
| 1877670 | C | T | 0.184 | 347449 | C | G | -1.611 | 7200 | A | C | -2.215 |
| 1877727 | C | T | 1.105 | 3479610 | T | C | -3.715 | 7246 | T | A | -3.895 |
| 1877909 | TGGGCC | GGGGG | 0.984 | 3479632 | G | A | -2.369 | 7361 | CG | TC | 0.393 |
| | GGT | CGGG | | | | | | | | | |
| 1878049 | G | T | -2.519 | 348178 | C | T | -3.631 | 7362 | G | C | 2.081 |
| 1878163 | C | G | -3.624 | 348662 | C | A | 3.1 | 7570 | C | T | -0.2999 |
| 1878591 | G | T | -3.624 | 355725 | G | C | -2.087 | 7570 | C | G | -0.4919 |
| 1879244 | TGGT | GGGG | -0.280 | 3570160 | A | G | -0.6069 | 760011 | G | C | -4.259 |
| 1879316 | C | T | -1.753 | 3570229 | C | G | -4.088 | 760306 | G | A | -0.7102 |
| 1879517 | G | T | -3.895 | 3987048 | G | A | -1.247 | 760309 | T | C | -3.281 |
| 1879632 | C | A | -3.715 | 403565 | T | G | 0.2367 | 760314 | G | T | -0.0489 |
| 1879654 | T | G | -3.416 | 403634 | G | T | -2.605 | 760912 | A | C | -2.814 |
| 1880070 | A | C | 0.297 | 404356 | T | G | 5.312 | 760978 | A | G | -1.879 |
| 1880381 | G | T | -3.718 | 405812 | T | C | -2.158 | 761074 | A | G | -0.1354 |
| 1880632 | G | A | -3.895 | 4124826 | A | C | -0.2273 | 761076 | T | G | -2.026 |
| 1881378 | CGG | CG | -0.831 | 4124832 | T | C | -1.77 | 761093 | G | T | -0.6438 |
| 1883766 | TGA | GGG | -0.854 | 4124884 | T | G | -0.4097 | 761093 | G | C | -0.1684 |
| 1883801 | C | T | -3.135 | 4238385 | G | A | 0.3323 | 761095 | T | C | -0.3701 |
| 1886422 | T | G | -0.610 | 4240562 | A | C | -2.672 | 761098 | GCCAA | GCCACC | -4.049 |
| | | | | | | | | | | AA | |
| 1886509 | T | G | -1.270 | 4241758 | G | A | -3.58 | 761100 | CAATTC | CA | -1.161 |
| | | | | | | | | | ATGGA | | |
| | | | | | | | | | (SEQ | | |
| | | | | | | | | | ID NO | | |
| | | | | | | | | | 001) | | |
| 2053666 | T | G | -1.399 | 4242704 | G | A | -4.619 | 761109 | G | T | -1.79 |
| 2101853 | A | G | -0.959 | 4243323 | G | A | -0.3996 | 761110 | A | T | -0.9756 |
| 2101915 | A | G | -0.048 | 4243430 | G | A | -3.121 | 761115 | A | C | -0.6562 |
| 2102207 | C | T | -1.120 | 4243730 | G | C | -3.739 | 761131 | G | A | -1.707 |
| 2102521 | G | A | -3.895 | 4243803 | A | G | -4.128 | 761135 | GACCCA | TACCTG | -3.282 |
| 2102615 | T | G | -3.895 | 4243830 | G | A | -1.421 | 761139 | C | T | -0.7426 |
| 2102638 | T | C | -3.807 | 4244069 | C | T | 0.7047 | 761139 | C | G | -0.6112 |
| 2102778 | C | A | -1.759 | 4244093 | C | T | -3.895 | 761139 | CA | GG | -3.895 |
| 2153813 | A | C | 0.268 | 4244286 | G | A | -3.994 | 761139 | CA | AG | -5.132 |
| 2154016 | C | A | -0.425 | 4244474 | G | A | -3.895 | 761140 | A | T | -0.1099 |
| 2154108 | C | T | -3.895 | 4244631 | A | C | -0.1459 | 761154 | TCGGC | CAGGG | -2.088 |
| 2154211 | AGG | AGGG | -2.562 | 4244815 | T | G | -3.624 | 761155 | C | G | -1.437 |
| 2154232 | A | G | -2.864 | 4245187 | C | A | -0.06857 | 761155 | C | T | -1.897 |
| 2154240 | C | T | -0.963 | 4245226 | A | G | -2.581 | 761161 | T | C | -0.3494 |
| 2154332 | A | T | -0.133 | 4245311 | C | G | -2.368 | 761729 | C | T | -1.759 |
| 2154354 | CAC | CC | -2.030 | 4246759 | C | G | -4.564 | 761828 | C | G | -0.7551 |
| 2154461 | CG | CTG | -2.695 | 4246791 | C | T | -2.22 | 761889 | G | C | -4.506 |
| 2154590 | T | C | -0.239 | 4247050 | C | T | -0.6455 | 762072 | A | G | -2.03 |
| 2154746 | CAA | CA | -3.814 | 4247429 | A | G | -0.7152 | 762399 | G | T | -3.404 |
| 2154761 | CGACGA | CG | -3.314 | 4247431 | G | A | -0.3476 | 762974 | G | C | -2.011 |
| | GGTCGT | | | | | | | | | | |
| | GG | | | | | | | | | | |
| | (SEQ | | | | | | | | | | |
| | ID NO | | | | | | | | | | |
| | 002) | | | | | | | | | | |
| 2154823 | AG | GC | -3.624 | 4247573 | G | A | -0.6191 | 763039 | C | T | -0.8927 |
| 2154834 | G | C | -3.416 | 4247574 | A | G | -2.04 | 763111 | A | C | -2.545 |
| 2154877 | C | T | -4.619 | 4247729 | G | A | -0.947 | 764992 | G | A | -4.249 |
| 2154973 | G | T | -2.030 | 4248184 | G | C | 3.302 | 766047 | C | T | -3.856 |
| 2154973 | G | A | -1.565 | 4248200 | A | C | -0.02467 | 766767 | A | C | -0.384 |
| 2155102 | T | C | 0.271 | 4248343 | G | A | -2.03 | 7776 | C | G | -0.6437 |
| 2155120 | C | A | -2.664 | 4248516 | T | C | -3.895 | 779316 | G | T | -1.611 |
| 2155167 | GC | TG | -3.292 | 4249323 | C | A | -0.211 | 781687 | A | G | -0.7727 |
| 2155167 | GC | AG | -3.141 | 4249327 | G | A | -2.964 | 781822 | A | G | -0.5346 |
| 2155167 | GC | CG | -2.978 | 4249545 | A | C | -3.895 | 781822 | A | T | -0.6246 |
| 2155168 | C | G | -5.426 | 4249746 | T | G | 2.37 | 7862 | C | T | -0.08349 |
| 2155168 | C | T | -4.725 | 4258251 | A | G | -0.3831 | 8017 | G | A | -3.735 |
| 2155168 | C | A | -4.005 | 4258832 | G | A | -1.714 | 8171 | G | T | 2.137 |
| 2155168 | CTGGT | GTGGC | -0.677 | 4259665 | G | C | -2.03 | 8303 | C | T | -3.895 |
| 2155169 | T | C | -3.082 | 4260684 | C | A | -4.025 | 8393 | T | C | -2.87 |
| 2155206 | G | C | -2.961 | 4269317 | A | C | -0.4345 | 8668 | C | G | -4.128 |
| 2155212 | C | G | -3.580 | 4308395 | G | A | -0.05994 | 8717 | C | G | -1.259 |
| 2155213 | C | G | -1.068 | 4326101 | T | C | -3.624 | 875532 | C | G | -1.611 |
| 2155217 | C | T | -2.780 | 4326282 | C | A | -1.611 | 891663 | T | C | -3.895 |
| 2155300 | G | A | -2.029 | 4326313 | G | A | -3.895 | 891865 | A | C | -3.289 |
| 2155351 | C | A | -3.895 | 4326737 | T | C | -0.9851 | 892128 | C | T | -2.928 |
| 2155416 | CG | CAG | -3.494 | 4326800 | A | G | -0.667 | 9276 | C | T | -2.848 |
| 2155418 | G | A | 0.230 | 4326980 | T | G | -2.604 | 9392 | C | T | -4.727 |
| 2155430 | T | G | -1.552 | 4327133 | T | G | -2.713 | 9437 | G | A | -4.128 |
| 2155478 | CG | CAG | -3.624 | 4327323 | G | A | -1.266 | 9719 | G | C | -3.725 |
| 9735 | C | T | -3.895 | | | | | | | | |

TABLE B
Rifampicin weighted positions; threshold value: -0.318

| POS | RN | SN | W | POS | RN | SN | W | POS | RN | SN | W |
|---|---|---|---|---|---|---|---|---|---|---|---|
| 107937 | ACC | ACCC | -1.079 | 2751953 | C | AG | 0.2904 | 5452 | G | A | -1.116 |
| 1084659 | T | G | 2.688 | 2751960 | A | G | 0.0804 | 575760 | G | T | -3.147 |
| 1085605 | C | T | -1.079 | 2752074 | T | G | -0.3468 | 576618 | ACGC | ACGCG | -0.5844 |
| | | | | | | | | | | C | |
| 1128074 | A | G | -0.190 | 2752122 | G | C | -0.04697 | 576660 | T | G | -1.079 |
| 1131738 | T | C | -3.434 | 2831532 | G | T | 2.254 | 576710 | C | G | -0.1681 |
| 1132004 | A | C | -3.294 | 2875149 | T | G | 0.4523 | 61178 | C | T | -2.25 |
| 1237557 | A | C | -1.079 | 2876064 | T | C | -0.07538 | 615211 | T | G | 0.05838 |
| 1472069 | G | T | -1.079 | 2939020 | A | T | 1.899 | 616092 | G | T | -1.559 |
| 1472155 | C | A | -0.918 | 2939609 | C | A | -4.82 | 616196 | A | C | -2.11 |
| 1472359 | A | C | -0.070 | 2956293 | ACTGC | AC | 0.1812 | 61988 | C | T | -3.148 |
| 1472362 | C | T | -0.281 | 2956379 | C | T | -1.773 | 6302 | G | T | -0.1179 |
| 1472674 | T | C | -2.962 | 2956571 | G | C | -1.079 | 6346 | C | T | 6.218 |
| 1473246 | A | G | -1.005 | 3074424 | C | A | -0.2901 | 6576 | G | A | 2.157 |
| 1474686 | A | G | -0.270 | 3272997 | A | G | -0.1621 | 6579 | C | A | -1.369 |
| 1474833 | G | T | -2.733 | 3273214 | C | A | -0.6575 | 6794 | A | G | -5.12 |
| 1474900 | G | T | -0.255 | 3296271 | T | C | -3.054 | 6866 | C | T | 2.236 |
| 1475751 | C | T | -1.596 | 3296736 | T | G | -0.3921 | 7335 | C | T | -1.429 |
| 1488086 | C | G | 0.730 | 3296843 | A | G | 1.632 | 7362 | G | C | 2.091 |
| 1502745 | A | G | -2.514 | 3296925 | G | C | -0.04796 | 7495 | GCC | GCCC | -3.038 |
| 1674026 | A | C | 5.568 | 3296928 | CCACCG | CC | -2.962 | 7521 | G | T | -2.164 |
| | | | | | CCAGC | | | | | | |
| | | | | | (SEQ | | | | | | |
| | | | | | ID NO | | | | | | |
| | | | | | 003) | | | | | | |
| 1674481 | T | G | -0.792 | 3388838 | G | A | 3.358 | 7570 | C | T | -0.3307 |
| 1674709 | T | C | -0.656 | 345504 | A | C | -3.172 | 7570 | C | G | -0.2374 |
| 1674769 | G | T | 1.091 | 346563 | C | T | -2.233 | 7585 | G | C | 0.1589 |
| 1674782 | T | C | -0.104 | 3466825 | G | C | 0.825 | 760306 | G | A | -0.52 |
| 1674966 | T | G | 0.110 | 3467118 | T | C | -1.273 | 760308 | G | A | -0.3262 |
| 1759252 | G | T | -0.251 | 347221 | G | C | -1.208 | 760314 | G | T | -4.974 |
| 1816240 | G | T | 0.452 | 347393 | C | T | -3.513 | 760483 | A | G | -1.522 |
| 1816487 | G | T | -0.499 | 347449 | C | G | -1.079 | 761095 | T | C | -4.684 |
| 1817357 | T | G | -2.585 | 348413 | C | T | 4.728 | 761095 | TGA | CGG | -1.903 |
| 1833906 | A | T | -1.545 | 348662 | C | A | 2.599 | 761095 | T | G | -0.07896 |
| 1834177 | A | C | -0.178 | 349054 | C | T | -1.716 | 761098 | GCCAA | GCCACC | -4.625 |
| | | | | | | | | | | AA | |
| 1849861 | G | A | -0.412 | 349071 | A | G | -1.647 | 761100 | C | A | -2.983 |
| 1850293 | T | A | -0.107 | 349151 | A | G | 2.364 | 761100 | CAATTC | CA | -3.292 |
| | | | | | | | | | ATGGA | | |
| | | | | | | | | | (SEQ ID | | |
| | | | | | | | | | NO 001) | | |
| 1851932 | C | A | -0.168 | 349205 | A | C | -3.172 | 761101 | A | C | -4.14 |
| 1875440 | A | C | -0.923 | 3514463 | G | A | 1.787 | 761101 | A | T | -4.365 |
| 1875499 | G | T | -0.521 | 355692 | T | G | 0.8223 | 761102 | ATTCA | ATTCTT | -3.929 |
| | | | | | | | | | | CA | |
| 1875562 | G | C | -0.064 | 3569664 | T | C | -0.04971 | 761104 | TCATGG | TC | -2.545 |
| | | | | | | | | | AC | | |
| 1875902 | T | C | -1.079 | 3569739 | C | G | -0.2074 | 761107 | TGGACC | TG | -4.712 |
| | | | | | | | | | AG | | |
| 1876420 | T | G | 0.107 | 3570229 | C | G | -0.9203 | 761107 | TGG | GGT | -1.868 |
| 1876531 | G | A | -0.558 | 3836358 | G | C | 1.534 | 761108 | GG | TT | -1.729 |
| 1877866 | G | C | 3.503 | 403565 | T | G | 0.034 | 761108 | GGACC | GC | -4.555 |
| 1877947 | G | A | -2.402 | 403828 | C | T | -3.38 | 761109 | G | T | -4.337 |
| 1878128 | A | G | -0.405 | 404356 | T | G | 4.362 | 761109 | GA | TT | -3.89 |
| 1878238 | T | G | -0.607 | 405404 | C | T | -3.755 | 761109 | GA | CT | -2.655 |
| 1878416 | A | C | -0.770 | 405479 | G | A | -3.034 | 761110 | A | T | -4.824 |
| 1878817 | CGGGG | CGGGG | 0.860 | 4125415 | A | G | -0.7979 | 761110 | A | G | -0.7976 |
| | GGC | GC | | | | | | | | | |
| 1879405 | G | T | -1.878 | 4229167 | T | C | -0.02295 | 761127 | TC | CA | -2.151 |
| 1879716 | G | T | 1.328 | 4240678 | T | G | -0.1713 | 761127 | TC | AT | -0.2862 |
| 1879823 | A | C | -0.214 | 4241292 | T | G | 7.094 | 761128 | C | T | -4.343 |
| 1880329 | A | C | -0.104 | 4243217 | C | G | 1.905 | 761128 | C | G | -2.946 |
| 1882443 | C | T | -0.062 | 4243379 | C | A | -0.3618 | 761131 | G | A | -0.7445 |
| 1882933 | T | A | -0.770 | 4244850 | C | G | 0.2329 | 761134 | TGACCC | GGCCC | -5.407 |
| | | | | | | | | | ACAA | CA | |
| | | | | | | | | | (SEQ ID | | |
| | | | | | | | | | NO 004) | | |
| 1883716 | T | G | 0.112 | 4245001 | T | C | -0.1319 | 761135 | GACCCA | TACCTG | -3.767 |
| 1883736 | GGT | GGG | -3.902 | 4245055 | C | A | -0.1318 | 761139 | C | T | -4.49 |
| 1883750 | T | G | -0.215 | 4245226 | A | G | 0.07515 | 761139 | C | G | -4.887 |
| 1886509 | T | G | -1.767 | 4245311 | C | G | 1.016 | 761139 | C | A | -3.276 |
| 1917851 | C | A | -0.435 | 4247429 | A | G | -1.17 | 761139 | CA | TG | -4.249 |
| 1918019 | T | C | 0.946 | 4247431 | G | C | -0.2875 | 761139 | CA | GG | -4.172 |
| 1918340 | C | T | -1.828 | 4247431 | G | A | -0.9326 | 761139 | CA | AG | -5.39 |
| 2054227 | T | G | -0.431 | 4247431 | G | T | -0.28 | 761140 | A | G | -4.607 |
| 2102289 | C | T | -2.168 | 4247507 | T | C | 0.4568 | 761140 | A | T | -4.12 |
| 2133859 | C | T | -3.911 | 4247574 | A | C | -0.06571 | 761141 | C | A | -0.7741 |
| 2153819 | C | G | -0.926 | 4247729 | G | A | -0.1473 | 761154 | TCGGC | CAGGG | -3.688 |
| 2154108 | C | T | -1.744 | 4247730 | G | C | -0.3736 | 761154 | TC | CA | -3.204 |
| 2154158 | ACTCGG | AGGCCT | 2.117 | 4247747 | T | C | -1.89 | 761155 | C | G | -4.885 |
| | AG | CG | | | | | | | | | |
| 2154214 | A | G | -0.542 | 4248003 | A | G | -0.7354 | 761155 | C | T | -5.572 |
| 2154695 | C | A | -0.846 | 4249272 | A | C | -0.2619 | 761155 | CG | TT | -2.277 |
| 2154834 | G | C | -0.819 | 4249327 | G | A | -4.151 | 761155 | CG | TC | -2.021 |
| 2154857 | C | G | -0.261 | 4258428 | C | G | 0.03848 | 761161 | T | C | -3.604 |
| 2155167 | GC | CG | -0.190 | 4259573 | A | C | 0.1593 | 761167 | CCGGC | CCGGT | -2.363 |
| | | | | | | | | | GGT | | |
| 2155168 | C | G | -1.490 | 4269162 | C | A | -0.6126 | 761277 | A | T | -3.084 |
| 2155168 | C | T | -1.281 | 4269722 | C | A | 0.5053 | 761278 | T | C | -1.915 |
| 2155258 | C | A | 7.345 | 4269848 | T | C | -3.508 | 761279 | C | G | -4.764 |
| 2155430 | T | G | -1.000 | 4308395 | G | A | -0.07952 | 761827 | A | G | -0.3675 |
| 2155636 | A | G | -0.100 | 4308402 | A | G | -0.8195 | 762399 | G | T | -3.258 |
| 2155648 | T | C | -0.145 | 4308881 | C | T | -0.1779 | 764181 | A | G | -0.7252 |
| 2155746 | G | A | 2.527 | 4326282 | C | A | -1.079 | 764323 | G | T | 2.117 |
| 2155819 | T | C | -0.087 | 4326341 | G | A | 1.056 | 764617 | C | T | 2.167 |
| 2279407 | G | A | -0.459 | 4326426 | CAAAAA | CAAAAG | -0.5703 | 764749 | G | A | 4.861 |
| | | | | | G | | | | | | |
| 2288730 | G | T | 3.271 | 4326611 | G | C | 2.636 | 767123 | G | T | -0.1358 |
| 2288740 | T | G | 4.684 | 4326831 | G | A | 3.238 | 7685 | G | A | 1.533 |
| 2288813 | GGCCG | GGCCG | 1.712 | 4326980 | T | G | -0.006595 | 778953 | T | C | -3.254 |
| | TCTGGC | TCTGGC | | | | | | | | | |
| | G (SEQ | GCCGTC | | | | | | | | | |
| | ID NO | TGGCG | | | | | | | | | |
| | 005) | (SEQ ID | | | | | | | | | |
| | | NO 006) | | | | | | | | | |
| 2288850 | ACC | ACCC | -0.105 | 4327241 | C | T | 0.9404 | 779181 | CGGGG | CGGGG | -0.0104 |
| | | | | | | | | | GGA | GA | |
| 2288868 | A | C | -0.059 | 4327294 | G | T | 1.008 | 779316 | G | T | -1.079 |
| 2288868 | A | T | -0.449 | 4328212 | CGGGG | CGGGGT | -0.6804 | 781822 | A | G | -0.162 |
| | | | | | GT | | | | | | |
| 2288932 | TGTACG | TG | -0.029 | 4407620 | A | G | -0.4966 | 781937 | C | T | -0.6332 |
| 2289164 | CGC | CC | 1.935 | 4407677 | G | A | -0.4789 | 782750 | T | C | -2.233 |
| 2289223 | C | G | 0.680 | 4407734 | C | T | 1.419 | 783387 | C | T | -4.902 |
| 2412085 | A | C | 0.154 | 4407847 | G | T | -0.4189 | 783783 | G | A | -0.04834 |
| 2518919 | G | A | -0.126 | 4407927 | T | G | -0.09332 | 7914 | C | A | -0.5087 |
| 2620945 | G | T | -1.268 | 4407940 | A | G | -0.6359 | 7960 | T | G | 1.694 |
| 2693848 | G | T | -1.537 | 4407973 | A | G | -0.5723 | 796747 | T | G | -0.7455 |
| 2693851 | G | T | -3.415 | 491405 | T | A | 0.4985 | 8147 | C | G | -3.338 |
| 2693853 | T | G | -1.972 | 496401 | T | C | 1.001 | 8435 | C | G | -1.273 |
| 2693952 | A | C | 0.639 | 497243 | G | C | 0.1293 | 8721 | C | G | -1.224 |
| 2693966 | A | C | -1.868 | 497491 | G | A | -0.1006 | 875005 | T | G | 0.07587 |
| 2714465 | G | T | -1.079 | 513282 | G | A | -1.079 | 875532 | C | G | -0.8313 |
| 2714566 | AGGG | GGGCG | 0.284 | 513468 | T | G | 5.44 | 891648 | C | G | 0.08224 |
| | | GGG | | | | | | | | | |
| 2715072 | C | T | -0.277 | 514344 | G | A | -0.0399 | 891788 | C | G | 1.412 |
| 2726338 | T | G | 0.029 | 5300 | G | A | -3.867 | 9276 | C | T | -1.988 |
| 273690 | C | T | -0.185 | 5311 | G | A | -0.5311 | 930984 | T | C | 0.4523 |
| 274030 | C | T | -1.916 | 538916 | G | T | 0.4523 | 9395 | G | A | 0.7507 |
| 2751605 | ACGCAA | AC | -0.266 | 539228 | C | G | 0.4523 | 9788 | C | T | -1.861 |
| | AAGCTC | | | | | | | | | | |
| | CCAAAT | | | | | | | | | | |
| | CCGACC | | | | | | | | | | |
| | GGATTT | | | | | | | | | | |
| | GGGAG | | | | | | | | | | |
| | CTTTTG | | | | | | | | | | |
| | CGTCTT | | | | | | | | | | |
| | TTCGCG | | | | | | | | | | |
| | GTGGTC | | | | | | | | | | |
| | AGCCG | | | | | | | | | | |
| | CGGCG | | | | | | | | | | |
| | GCC | | | | | | | | | | |
| | (SEQ ID | | | | | | | | | | |
| | NO 007) | | | | | | | | | | |
| 2751923 | A | G | -4.616 | 541439 | G | A | -3.294 | 9808 | C | T | -0.2397 |
| 9846 | C | G | -3.254 | | | | | | | | |

TABLE C
Ethambutol weighted positions: threshold value -1.775

| POS | RN | SN | W | POS | RN | SN | W | POS | RN | SN | W |
|---|---|---|---|---|---|---|---|---|---|---|---|
| 107937 | ACC | ACCC | -2.458 | 2289031 | G | T | -1.399 | 4269848 | T | C | -2.555 |
| 1084236 | G | A | 0.205 | 2289032 | CGGTG | CGGTG | 0.9001 | 4308395 | G | A | -0.0269 |
| | | | | | | GTG | | | | | |
| 1084280 | A | G | 0.548 | 2289038 | C | A | -0.657 | 4326113 | G | A | -0.7514 |
| 1084792 | GGT | GGG | 0.272 | 2289061 | T | G | -1.297 | 4326132 | TGG | TG | -2.611 |
| 1085605 | C | T | -2.245 | 2289068 | G | T | -0.867 | 4326282 | C | A | -2.458 |
| 1128074 | A | G | -1.507 | 2289068 | G | C | -0.3289 | 4326341 | G | A | 0.2513 |
| 1128601 | T | C | 0.778 | 2289090 | T | C | 0.9958 | 4326350 | G | A | -0.2988 |
| 1128951 | G | A | 3.119 | 2289096 | T | C | -0.1176 | 4326365 | T | G | -0.2287 |
| 1131944 | T | C | 0.273 | 2289097 | C | T | 0.6811 | 4326547 | CTT | CT | -2.493 |
| 1132004 | A | C | -4.292 | 2289100 | T | C | -0.06007 | 4326593 | C | T | 1.046 |
| 1237205 | A | C | -0.435 | 2289144 | T | A | -1.144 | 4326613 | GTT | GT | 2.72 |
| 1237557 | A | C | -2.458 | 2289156 | CGG | CGGG | -2.471 | 4326621 | G | T | 3.636 |
| 1416260 | A | G | -0.505 | 2289164 | CGC | CC | 1.01 | 4326801 | GCT | GCCT | -0.04766 |
| 1461119 | T | G | 0.693 | 2289193 | C | T | 1.17 | 4326943 | G | C | 0.5908 |
| 1471921 | GGTCTC | GGTCTC | 0.271 | 2289194 | ACC | ACCC | -2.965 | 4326980 | T | G | -1.223 |
| | TTCGGA | TTCGGA | | | | | | | | | |
| | GATACT | GATACT | | | | | | | | | |
| | C (SEQ | GTCTCT | | | | | | | | | |
| | ID NO | TCGGAG | | | | | | | | | |
| | 008) | ATACTC | | | | | | | | | |
| | | (SEQ ID | | | | | | | | | |
| | | NO 009) | | | | | | | | | |
| 1472069 | G | T | -2.458 | 2289216 | A | G | -0.1347 | 4326981 | G | A | 2.192 |
| 1472155 | C | A | -1.886 | 2289226 | T | G | 0.2625 | 4326983 | G | A | -2.551 |
| 1472705 | G | T | 2.482 | 2289231 | A | G | -0.6494 | 4327065 | A | G | -0.7151 |
| 1472723 | G | A | -0.720 | 2289252 | T | G | 2.247 | 4327133 | T | G | 0.7011 |
| 1472920 | GTT | GTTT | -4.479 | 2289252 | T | C | 0.2179 | 4327193 | A | C | 1.22 |
| 1472970 | C | T | -0.558 | 2411092 | G | T | -4.486 | 4327323 | G | A | -0.5768 |
| 1473246 | A | G | -0.662 | 2504627 | G | A | -0.2572 | 4327346 | C | A | -0.2981 |
| 1473254 | A | C | -1.558 | 2518809 | A | G | -0.01745 | 4327448 | A | C | -0.5885 |
| 1473835 | G | A | 0.563 | 2518919 | G | A | -0.2937 | 4328212 | CGGGG | CGGGG | -1.818 |
| | | | | | | | | | GT | T | |
| 1474200 | ATT | AT | 0.428 | 2620945 | G | T | -2.675 | 4399047 | G | C | -0.1088 |
| 1475395 | T | C | -3.499 | 2693848 | G | T | -2.34 | 4399252 | A | G | -0.2229 |
| 1475778 | G | A | -2.152 | 2693851 | G | T | -4.797 | 4399261 | GCA | GCC | -1.756 |
| 1476105 | G | A | -0.616 | 2693853 | T | G | -2.529 | 4407794 | G | A | -1.677 |
| 1476600 | A | T | 1.003 | 2694792 | G | A | 0.9544 | 4407866 | C | A | 0.7704 |
| 1487556 | C | T | 0.752 | 2694805 | C | A | 1.707 | 4407927 | T | G | -0.05012 |
| 1501971 | C | A | 1.572 | 2726136 | C | T | 0.2232 | 4407949 | T | G | 0.357 |
| 157129 | C | T | 0.290 | 2726338 | T | G | 0.05506 | 4408124 | C | G | 1.028 |
| 157214 | G | A | -0.092 | 2726341 | TGTTC | GGGTT | 1.021 | 4408138 | T | G | -0.01963 |
| 157555 | T | G | 0.490 | 2726341 | TGTTC | GGTTTG | -3.828 | 4408150 | A | T | -0.4527 |
| | | | | | | GTTC | | | | | |
| | | | | | | (SEQ ID | | | | | |
| | | | | | | NO 010) | | | | | |
| 1674048 | G | A | -0.319 | 273099 | A | G | -0.1801 | 490696 | T | G | 0.6564 |
| 1674248 | T | C | -0.000 | 273508 | T | C | -0.1608 | 495040 | G | A | -4.984 |
| 1674356 | A | G | -3.716 | 274030 | C | T | -3.071 | 495757 | T | C | -1.524 |
| 1674417 | C | T | 1.584 | 274141 | C | T | -3.411 | 496386 | G | A | 0.643 |
| 1674709 | T | C | -1.567 | 2751677 | G | A | 0.4571 | 496666 | C | T | -1.783 |
| 1674782 | T | C | -0.637 | 2751953 | C | AG | -0.05598 | 497389 | CCAG | CG | -3.33 |
| 1674837 | C | T | 0.005 | 2751960 | A | G | 0.001925 | 512358 | GCG | GCCG | -0.8782 |
| 1719904 | C | A | 0.366 | 2751974 | G | A | -0.1027 | 513468 | T | G | 1.025 |
| 1719949 | T | C | -1.879 | 2751974 | GCCCG | ACCCC | 0.938 | 514344 | G | A | -1.56 |
| 1758384 | G | A | 1.314 | 2751984 | T | C | 0.5663 | 514808 | T | G | -0.5121 |
| 1758588 | A | G | -0.250 | 2752122 | G | C | -0.008562 | 515287 | G | A | 1.199 |
| 1758596 | T | C | -2.607 | 2876061 | A | C | 0.1961 | 5311 | G | A | -1.639 |
| 1759252 | G | T | -0.538 | 2937360 | T | C | 0.7362 | 538656 | G | T | 1.057 |
| 1799759 | T | G | -0.271 | 2939372 | AG | AC | 6.916 | 538681 | C | A | 0.9804 |
| 1816204 | A | G | 1.611 | 2939373 | G | C | 0.04322 | 540353 | A | C | -0.0419 |
| 1833695 | C | G | 2.051 | 2939609 | C | A | -5.374 | 541439 | G | A | -4.584 |
| 1833763 | C | T | -3.473 | 2940725 | G | C | 0.4757 | 5530 | C | G | -2.373 |
| 1834177 | A | C | -0.110 | 2940748 | T | C | 0.5282 | 575503 | G | A | -3.856 |
| 1834187 | G | C | 1.934 | 2940758 | A | C | 0.05618 | 575538 | A | G | -0.06581 |
| 1849861 | G | A | -0.149 | 2955203 | C | T | -3.462 | 576016 | A | G | -2.84 |
| 1850309 | A | C | 0.659 | 2956336 | T | C | 0.03141 | 576400 | C | T | 3.759 |
| 1875457 | T | G | 2.155 | 3020515 | G | T | 0.3948 | 576660 | T | G | -2.458 |
| 1875550 | GA | TT | 0.757 | 3073685 | C | G | 0.1367 | 576705 | A | G | -1.258 |
| 1875550 | GA | TA | 1.051 | 3272997 | A | G | -0.5377 | 576710 | C | G | -0.03793 |
| 1875576 | G | A | -0.415 | 3273107 | C | A | 0.4061 | 576718 | CGA | GGG | 2.659 |
| 1875587 | GGT | CGG | -0.074 | 3296271 | T | C | -4.87 | 576742 | GGT | GGG | -3.311 |
| 1875589 | T | G | 0.016 | 3296300 | C | A | 0.5481 | 5856 | T | G | -2.881 |
| 1875618 | TTTTG | GTTTT | 1.627 | 3296437 | T | G | -0.2611 | 60373 | T | A | 1.2 |
| 1875629 | A | G | -0.956 | 3296639 | ACC | AC | -0.05812 | 60551 | A | C | 0.8874 |
| 1875670 | C | A | 0.582 | 3296736 | T | G | 0.1371 | 6144 | A | G | -0.3711 |
| 1875902 | T | C | -2.458 | 3296753 | G | C | -3.645 | 615211 | T | G | 0.02088 |
| 1875915 | C | G | 0.975 | 3296792 | G | C | -1.356 | 61543 | A | T | -7.939 |
| 1876332 | GGGC | CGGG | 0.663 | 3296843 | A | G | 0.7089 | 616196 | A | C | -2.472 |
| 1876420 | T | G | 0.201 | 3296848 | G | A | -0.3835 | 61898 | C | G | -1.339 |
| 1876531 | G | A | -1.361 | 3296962 | G | C | -5.089 | 61924 | C | T | 1.491 |
| 1877298 | C | G | 0.092 | 3296999 | T | C | 1.375 | 61988 | C | T | -3.664 |
| 1877315 | A | G | 0.057 | 3297010 | G | A | -4.714 | 62801 | C | A | 1.493 |
| 1877326 | G | C | 0.297 | 3297018 | C | T | 3.912 | 648774 | C | T | -1.394 |
| 1877727 | C | T | 1.119 | 3297704 | G | A | 0.706 | 648992 | C | G | -0.08657 |
| 1877831 | A | G | -0.008 | 3326134 | T | C | 0.2469 | 6576 | G | A | -3.877 |
| 1877909 | TGGGC | GGGGG | 0.132 | 345504 | A | C | -4.584 | 6734 | A | G | -0.2115 |
| | CGGT | CGGG | | | | | | | | | |
| 1877947 | G | A | -2.169 | 346563 | C | T | -3.199 | 6794 | A | G | -5.591 |
| 1877984 | A | G | 0.916 | 3466562 | G | T | -0.9245 | 6918 | ATGTG | TTGTT | 0.969 |
| 1878570 | T | G | -3.931 | 347221 | G | C | -2.098 | 6973 | G | A | -0.2529 |
| 1878619 | GT | GG | 1.288 | 347256 | C | T | -5.216 | 7268 | C | T | 1.17 |
| 1878723 | T | G | -0.005 | 347393 | C | T | -5.038 | 7362 | G | C | 2.115 |
| 1879531 | GATG | GG | 3.735 | 347449 | C | G | -2.458 | 7521 | G | T | -0.5012 |
| 1879717 | GGCTG | GG | -0.275 | 348381 | C | T | -0.2188 | 7570 | C | T | -0.3029 |
| | GTCGTT | | | | | | | | | | |
| | G (SEQ | | | | | | | | | | |
| | ID NO | | | | | | | | | | |
| | 011) | | | | | | | | | | |
| 1879849 | GC | GG | 1.393 | 349071 | A | G | -2.285 | 7582 | ACAG | CCAC | -0.4839 |
| 1879858 | T | G | 1.752 | 349205 | A | C | -4.584 | 7584 | AG | GC | -2.188 |
| 1879864 | GC | GG | 0.896 | 349520 | A | G | -0.6133 | 760209 | A | G | 1.788 |
| 1879870 | TATTGG | GATTTG | 2.520 | 354606 | C | T | -2.369 | 760308 | G | A | -0.7744 |
| | GGT | GGG | | | | | | | | | |
| 1880234 | CGG | CG | -3.118 | 355004 | C | A | -2.11 | 760480 | G | C | 0.9946 |
| 1880402 | G | T | -1.094 | 355082 | C | A | -0.005737 | 760567 | C | T | 0.7285 |
| 1881713 | A | C | -0.252 | 355692 | T | G | 1.374 | 761045 | C | T | -0.05706 |
| 1882618 | T | G | 1.855 | 355860 | C | G | -2.955 | 761076 | T | G | -0.08802 |
| 1882653 | T | G | 0.100 | 3569344 | G | C | 5.503 | 761093 | G | T | -0.4355 |
| 1882958 | T | G | 0.785 | 3569759 | C | A | -0.3351 | 761100 | C | A | 1.266 |
| 1883377 | G | C | 3.029 | 3570229 | C | G | -1.765 | 761101 | A | T | -0.1552 |
| 1883716 | T | G | 0.158 | 3570265 | C | G | 1.228 | 761108 | GG | CT | 1.085 |
| 1883784 | C | G | -1.139 | 3570291 | C | A | 1.073 | 761109 | G | T | -0.9329 |
| 1883794 | A | C | 1.029 | 3570298 | A | C | 0.1995 | 761109 | GA | TT | -0.6942 |
| 1884260 | G | A | 1.045 | 3835223 | C | T | -2.399 | 761110 | A | T | -0.488 |
| 1917851 | C | A | -5.003 | 3836116 | G | A | -0.0129 | 761127 | TC | AT | 6.353 |
| 1917998 | G | T | -3.021 | 3986902 | G | A | -0.3509 | 761128 | C | G | -0.2549 |
| 1918010 | C | G | 0.736 | 3986931 | C | A | -0.4018 | 761131 | G | A | -1.003 |
| 1918019 | T | C | -1.416 | 403920 | C | T | -0.2228 | 761135 | GACCCA | TACCTG | -1.153 |
| 1918164 | GGCGTT | GGCGTT | 0.864 | 404132 | C | T | -0.3694 | 761139 | C | T | -0.3809 |
| | CG | CGCGTT | | | | | | | | | |
| | | CG (SEQ | | | | | | | | | |
| | | ID NO | | | | | | | | | |
| | | 012) | | | | | | | | | |
| 1918169 | T | G | -5.282 | 404356 | T | G | 0.4043 | 761139 | C | G | -0.6814 |
| 1918336 | CGG | CG | -1.298 | 405404 | C | T | -1.914 | 761139 | CA | GG | -1.266 |
| 1918340 | C | T | -2.252 | 405479 | G | A | -5.093 | 761140 | A | T | -1.023 |
| 2053562 | T | C | 2.979 | 4124351 | C | G | -4.584 | 761154 | TCGGC | CAGGG | -0.2556 |
| 2053589 | C | T | -0.081 | 4125386 | C | G | -0.774 | 761155 | C | G | -0.9029 |
| 2101853 | A | G | -3.826 | 4125415 | A | G | -0.5558 | 761155 | C | T | -0.8579 |
| 2101965 | C | T | 1.080 | 4238963 | C | T | -0.2097 | 761161 | T | C | -0.9807 |
| 2102120 | T | C | -0.298 | 4239274 | C | G | -0.5186 | 761244 | A | G | 1.143 |
| 2102289 | C | T | -2.320 | 4239476 | G | T | 0.6963 | 761268 | A | G | -2.496 |
| 2102696 | A | G | -1.348 | 4240661 | T | C | 0.9266 | 761292 | G | A | -0.1669 |
| 2102849 | T | C | -0.694 | 4240678 | T | G | -0.02568 | 761419 | C | T | 0.8166 |
| 2102912 | G | C | -2.505 | 4242075 | G | A | 0.008024 | 761461 | G | A | 3.177 |
| 2102949 | T | C | -0.975 | 4242435 | T | G | 0.7909 | 761528 | C | A | 0.6516 |
| 2133859 | C | T | -3.169 | 4243217 | C | G | 0.822 | 761583 | C | T | -0.1214 |
| 2133987 | C | T | -0.393 | 4243221 | C | T | 2.582 | 761973 | C | G | 0.1424 |
| 2153742 | A | G | 0.547 | 4243346 | A | G | -0.1444 | 762399 | G | T | -0.3968 |
| 2153819 | C | G | -2.132 | 4243667 | C | G | 1.301 | 762434 | T | G | 0.005517 |
| 2154108 | C | T | -1.962 | 4244631 | A | C | 0.4376 | 762967 | A | G | -1.443 |
| 2154232 | A | G | 0.100 | 4245009 | G | A | 1.117 | 7632 | G | A | 1.974 |
| 2154329 | G | A | -4.417 | 4246662 | T | C | -1.364 | 764181 | A | G | -2.492 |
| 2154695 | C | A | -0.306 | 4247399 | A | C | 0.7902 | 764673 | A | C | 2.934 |
| 2154857 | C | G | -0.039 | 4247402 | T | G | 0.5033 | 764744 | C | T | -0.02669 |
| 2154977 | C | T | -0.000 | 4247429 | A | G | -4.691 | 764924 | G | A | -2.407 |
| 2155163 | T | C | -0.347 | 4247429 | A | C | -2.975 | 764948 | T | G | -0.6006 |
| 2155167 | GC | CG | -0.323 | 4247431 | G | C | -3.305 | 765842 | A | G | -2.063 |
| 2155168 | C | G | -0.893 | 4247431 | G | A | -4.054 | 766198 | C | A | 2.744 |
| 2155217 | C | T | 0.629 | 4247431 | G | T | -3.479 | 766466 | G | C | -0.1748 |
| 2155301 | T | G | -0.596 | 4247469 | A | C | -4.072 | 766485 | T | C | -0.7597 |
| 2155430 | T | G | -2.185 | 4247469 | A | G | -3.855 | 7776 | C | G | -1.169 |
| 2155648 | T | G | 0.400 | 4247553 | G | C | -3.215 | 779316 | G | T | -2.458 |
| 2155741 | C | G | -0.662 | 4247574 | A | C | 0.4781 | 781687 | A | G | -0.5226 |
| 2155801 | C | T | -0.631 | 4247728 | G | C | -0.93 | 781880 | C | T | -0.6199 |
| 2288718 | ATCTCC | ATCTCC | -1.376 | 4247729 | G | A | -3.111 | 784696 | C | T | 4.911 |
| | T | TCTCCT | | | | | | | | | |
| | | (SEQ ID | | | | | | | | | |
| | | NO 013) | | | | | | | | | |
| 2288730 | G | T | 2.555 | 4247729 | G | T | -0.1692 | 7862 | C | T | -0.5825 |
| 2288739 | G | C | 0.943 | 4247730 | G | C | -3.307 | 7892 | GGCC | AGCT | -0.3082 |
| 2288771 | CA | CTA | 0.479 | 4247730 | G | A | -3.51 | 7914 | C | A | -0.4223 |
| 2288779 | C | T | -0.570 | 4247738 | G | C | -0.7601 | 7965 | C | T | -0.003783 |
| 2288782 | T | C | -0.155 | 4247999 | G | A | 0.2457 | 796747 | T | G | -0.07686 |
| 2288805 | G | A | -0.503 | 4248002 | C | A | -3.278 | 797074 | T | G | -0.3442 |
| 2288813 | GGCCG | GGCCG | -0.633 | 4248003 | A | G | -4.157 | 8075 | G | T | -3.391 |
| | TCTGGC | TCTGGC | | | | | | | | | |
| | G (SEQ | GCCGTC | | | | | | | | | |
| | ID NO | TGGCG | | | | | | | | | |
| | 005) | (SEQ ID | | | | | | | | | |
| | | NO 006) | | | | | | | | | |
| 2288827 | C | G | 1.977 | 4248003 | A | C | -0.7207 | 8164 | C | A | 1.334 |
| 2288830 | A | G | -0.309 | 4248024 | A | C | -0.6607 | 8364 | A | G | 2.04 |
| 2288844 | A | G | -0.085 | 4249246 | A | C | -0.5118 | 8435 | C | G | -2.805 |
| 2288852 | CACATC | CC | 0.207 | 4249518 | A | G | -4.393 | 8486 | T | C | 1.474 |
| 2288895 | AGT | AGGT | 0.084 | 4257766 | C | T | 0.1822 | 875005 | T | G | 0.03749 |
| 2288926 | A | G | -0.668 | 4259573 | A | C | 0.05949 | 875532 | C | G | -1.928 |
| 2288932 | TGTACG | TG | -0.258 | 4260099 | T | G | -0.2167 | 9162 | C | A | -0.077 |
| 2288938 | C | G | 0.547 | 4260769 | G | A | -0.3879 | 9224 | C | G | 1.146 |
| 2288942 | GGTG | GG | 8.470 | 4260790 | G | A | -0.5758 | 9289 | T | C | 1.116 |
| 2289029 | A | T | -1.555 | 4261150 | T | G | -0.3523 | 931017 | C | T | 1.532 |
| 2289030 | T | C | -0.009 | 4269722 | C | A | -0.7167 | 931443 | C | T | -4.892 |
| 2289031 | G | A | -3.441 | 4269731 | C | A | -0.01996 | 9395 | G | A | 0.6246 |
| 9591 | G | A | -0.260 | | | | | | | | |
| 9846 | C | G | -1.019 | | | | | | | | |

TABLE D
Pyrazinamide weighted positions; threshold value: -3.426

| POS | RN | SN | W | POS | RN | SN | W |
|---|---|---|---|---|---|---|---|
| 1128783 | G | A | 0.602 | 2288892 | AGC | AGGC | -0.471 |
| 1132004 | A | C | -4.250 | 2288900 | CGT | CT | -2.601 |
| 1136668 | G | T | -0.484 | 2288902 | T | G | -1.021 |
| 1136765 | G | A | -0.442 | 2288920 | C | A | -3.310 |
| 1237438 | T | G | -0.324 | 2288926 | A | G | -2.919 |
| 1237557 | A | C | -3.728 | 2288927 | GCC | GCCC | -3.542 |
| 1237627 | C | G | 0.051 | 2288930 | G | C | -5.261 |
| 1238171 | A | C | -0.126 | 2288932 | TGTACG | TG | -4.617 |
| 1416222 | A | G | -0.245 | 2288933 | G | C | -0.756 |
| 1416232 | ACA | GCC | -0.020 | 2288942 | GGTG | GG | -1.875 |
| 1471861 | T | A | -2.499 | 2288945 | G | T | -0.340 |
| 1471862 | T | G | 0.487 | 2288952 | C | T | 0.703 |
| 1471921 | GGTCTC | GGTCTC | -1.956 | 2288953 | C | G | -2.792 |
| | TTCGGA | TTCGGA | | | | | |
| | GATACT | GATACT | | | | | |
| | C (SEQ | GTCTCT | | | | | |
| | ID NO | TCGGAG | | | | | |
| | 008) | ATACTC | | | | | |
| | | (SEQ ID | | | | | |
| | | NO 009) | | | | | |
| 1472359 | A | C | -0.190 | 2288955 | TT | TAT | -2.250 |
| 1472633 | C | T | -0.908 | 2288956 | T | G | -1.013 |
| 1472723 | G | A | -0.315 | 2288971 | C | A | -3.768 |
| 1472970 | C | T | -4.142 | 2288985 | TCCA | TCCCCA | -1.879 |
| 1473246 | A | G | -0.570 | 2288992 | TGG | TGGG | -4.745 |
| 1473912 | C | G | -0.990 | 2289008 | GCC | GCCC | -1.606 |
| 1474261 | C | T | -2.342 | 2289029 | A | T | -4.454 |
| 1474317 | G | T | -1.874 | 2289030 | T | C | -4.156 |
| 1474900 | G | T | -5.093 | 2289031 | G | A | -4.341 |
| 1475395 | T | C | -2.860 | 2289031 | G | T | -3.591 |
| 1475762 | G | A | -0.395 | 2289039 | C | T | -3.710 |
| 1476105 | G | A | -3.552 | 2289049 | AA | ATA | -3.163 |
| 1501986 | T | G | -1.795 | 2289049 | A | G | 0.071 |
| 157174 | T | C | -0.229 | 2289056 | CG | CTG | -1.091 |
| 1674248 | T | C | -4.141 | 2289056 | CGG | CG | -1.672 |
| 1674356 | A | G | -2.694 | 2289090 | T | C | -0.032 |
| 1674448 | G | A | -3.253 | 2289090 | T | G | -1.036 |
| 1674709 | T | C | -3.944 | 2289091 | G | A | -4.137 |
| 1719904 | CAAA | AAAC | -0.335 | 2289091 | G | C | -3.877 |
| 1758278 | C | T | -0.565 | 2289092 | GAA | GA | -2.879 |
| 1758596 | T | C | -0.144 | 2289097 | C | T | -3.556 |
| 1759252 | G | T | 0.220 | 2289119 | G | C | -1.008 |
| 1799599 | A | G | 0.700 | 2289120 | TAG | TAAG | -3.826 |
| 1800040 | T | G | -2.718 | 2289137 | CAG | CG | -2.174 |
| 1816204 | A | G | 0.119 | 2289140 | G | C | -3.698 |
| 1833594 | C | A | -4.141 | 2289144 | T | A | -2.018 |
| 1833626 | A | G | -3.768 | 2289147 | C | A | -1.589 |
| 1834859 | G | A | -2.829 | 2289156 | CGG | CGGG | -3.535 |
| 1875457 | T | G | -0.152 | 2289162 | A | G | -2.038 |
| 1875499 | G | T | -0.185 | 2289163 | GCGCG | GC | -3.922 |
| | | | | | GCGCC | | |
| | | | | | ACC | | |
| | | | | | (SEQ ID | | |
| | | | | | NO 14) | | |
| 1875556 | G | C | -0.195 | 2289164 | CGC | CC | -1.396 |
| 1875562 | G | C | -0.020 | 2289167 | GGCGC | GC | -4.579 |
| | | | | | CACCG | | |
| | | | | | GTTACC | | |
| | | | | | GCCAG | | |
| | | | | | CGAGC | | |
| | | | | | CAC | | |
| | | | | | (SEQ ID | | |
| | | | | | NO 15) | | |
| 1875573 | C | G | -0.507 | 2289183 | GCCAG | GC | -1.072 |
| | | | | | CGAGC | | |
| | | | | | CAC | | |
| | | | | | (SEQ ID | | |
| | | | | | NO 16) | | |
| 1875576 | G | A | -0.096 | 2289213 | T | G | -0.937 |
| 1875587 | GGT | CGG | -1.771 | 2289214 | G | A | -3.974 |
| 1875670 | C | A | 0.232 | 2289219 | T | C | -0.245 |
| 1876531 | G | A | -0.110 | 2289223 | C | G | -0.355 |
| 1877831 | A | G | -0.474 | 2289226 | T | G | 0.061 |
| 1877909 | T | G | 0.230 | 2289231 | A | G | -0.681 |
| 1877913 | CCGGT | GCGGG | -3.149 | 2289239 | C | T | 0.672 |
| 1878542 | A | C | 0.430 | 2505553 | A | G | 0.428 |
| 1878586 | G | A | -2.746 | 2518151 | A | C | -0.680 |
| 1878829 | T | C | -0.342 | 2621145 | A | C | -3.889 |
| 1878834 | G | A | -4.075 | 2622182 | C | A | -0.674 |
| 1879399 | G | A | -0.081 | 2693848 | G | T | -2.146 |
| 1879517 | G | T | -4.576 | 2693851 | G | T | -4.381 |
| 1879531 | GATG | GG | 0.385 | 2693853 | T | G | -2.383 |
| 1879660 | G | A | 0.645 | 2693890 | G | C | -2.160 |
| 1879680 | G | T | -0.054 | 2694942 | G | A | 0.814 |
| 1879685 | T | G | -0.057 | 2714566 | A | G | -0.076 |
| 1879745 | T | G | -2.077 | 2714977 | T | G | -0.942 |
| 1879842 | TGGT | TGGG | -0.322 | 2715330 | C | T | -2.699 |
| 1879863 | G | T | 0.473 | 2726338 | TGGTGT | GGGGG | -0.663 |
| | | | | | TC | TTT | |
| 1879870 | TATTGG | GATTTG | 0.677 | 2726341 | T | G | 0.003 |
| | GGT | GGG | | | | | |
| 1879878 | TGC | GGG | -2.438 | 2726537 | G | A | -3.133 |
| 1880351 | A | C | -3.842 | 2751677 | G | A | -0.030 |
| 1882854 | C | G | -3.755 | 2751953 | C | AG | -1.092 |
| 1884305 | T | G | 0.244 | 2751987 | G | C | -0.079 |
| 1885336 | A | C | -0.792 | 2752005 | A | G | 0.908 |
| 1886027 | G | A | 0.888 | 2752046 | G | A | -1.246 |
| 1886509 | T | G | -3.147 | 2752059 | G | C | 0.805 |
| 2053562 | T | C | -0.025 | 2874086 | G | A | -4.576 |
| 2053589 | C | T | -0.456 | 2939372 | AG | GC | -0.939 |
| 2101915 | A | G | -2.476 | 2939373 | G | C | 0.796 |
| 2102188 | C | A | -0.724 | 2939609 | C | A | -4.682 |
| 2102417 | G | A | -0.695 | 2940725 | G | C | -2.193 |
| 2102466 | C | T | -1.330 | 2956336 | T | C | 0.282 |
| 2102486 | G | C | 0.931 | 3073685 | C | G | -3.552 |
| 2102536 | C | T | -0.978 | 3074073 | C | T | -3.754 |
| 2102912 | G | C | -1.983 | 3272251 | T | G | -0.023 |
| 2133987 | C | T | -4.614 | 3272659 | T | A | -0.203 |
| 2153899 | T | G | -0.935 | 3272997 | A | G | -0.302 |
| 2153906 | T | C | -0.608 | 3273164 | T | G | -0.547 |
| 2154332 | A | T | -1.945 | 3296639 | ACC | AC | -0.079 |
| 2154354 | CAC | CC | -4.377 | 3296753 | G | C | -2.859 |
| 2154695 | C | G | -3.217 | 3296843 | A | G | 0.067 |
| 2155168 | C | G | -0.619 | 3296848 | G | A | -0.559 |
| 2155168 | CTGGT | GTGGC | -4.792 | 3296925 | GCA | CCCGC | 0.345 |
| | | | | | | C | |
| 2155169 | T | C | -0.898 | 3296927 | A | C | -0.271 |
| 2155296 | G | A | -0.548 | 3296962 | G | C | -4.518 |
| 2155563 | G | T | -2.702 | 3297111 | T | G | -0.900 |
| 2155687 | T | C | -2.823 | 3326338 | A | C | 0.452 |
| 2288704 | C | A | -1.757 | 346205 | C | T | 0.576 |
| 2288714 | GCG | GCCG | -0.923 | 3466622 | G | T | -2.338 |
| 2288718 | ATCTCC | ATCTCC | -2.879 | 3470150 | G | A | -0.288 |
| | T | TCTCCT | | | | | |
| | | (SEQ ID | | | | | |
| | | NO 013) | | | | | |
| 2288724 | TCC | TCCC | -2.571 | 347221 | G | C | -0.478 |
| 2288727 | AGCG | AG | -3.784 | 347393 | C | T | -4.511 |
| 2288737 | CGGT | CGGGG | -2.763 | 3479470 | G | A | -4.576 |
| | | T | | | | | |
| 2288742 | G | A | -2.119 | 349071 | A | G | -2.248 |
| 2288746 | CGG | CG | -0.996 | 349151 | A | G | -1.023 |
| 2288757 | C | T | -3.637 | 349520 | A | G | -0.372 |
| 2288761 | CT | CGT | -1.481 | 3513724 | C | T | -2.950 |
| 2288771 | CA | CTA | -3.393 | 354606 | C | T | -1.962 |
| 2288778 | ACC | ACCC | -4.562 | 355860 | C | G | -2.409 |
| 2288780 | C | A | -3.380 | 3569344 | G | C | 0.132 |
| 2288782 | T | C | -4.109 | 3569447 | C | T | -0.848 |
| 2288785 | TGG | TGGG | -3.030 | 3570220 | T | C | 0.263 |
| 2288789 | CAA | CAAA | -2.181 | 3570258 | A | C | 0.253 |
| 2288791 | AGC | AGGC | -3.504 | 3570322 | T | C | -3.219 |
| 2288792 | GCC | GCCC | -4.518 | 3570337 | A | C | -0.057 |
| 2288797 | TGCGTA | TGCGTA | -1.880 | 3835223 | C | T | -0.491 |
| | CCGCG | CCGCG | | | | | |
| | (SEQ ID | CGTACC | | | | | |
| | NO 017) | GCG | | | | | |
| | | (SEQ ID | | | | | |
| | | NO 018) | | | | | |
| 2288813 | GGCCG | GGCCG | -2.171 | 3836116 | G | A | -1.477 |
| | TCTGGC | TCTGGC | | | | | |
| | G (SEQ | GCCGTC | | | | | |
| | ID NO | TGGCG | | | | | |
| | 005) | (SEQ ID | | | | | |
| | | NO 006) | | | | | |
| 2288820 | T | G | -0.520 | 403417 | C | A | -2.146 |
| 2288821 | G | A | -2.148 | 405479 | G | A | -4.688 |
| 2288823 | C | G | -1.872 | 4124351 | C | G | -4.511 |
| 2288827 | C | G | -3.564 | 4124906 | A | C | 0.072 |
| 2288834 | ATC | ATTC | -4.256 | 4238456 | G | A | -0.498 |
| 2288836 | C | T | -1.949 | 4239863 | A | G | -2.609 |
| 2288847 | CCGACC | CC | -4.499 | 4242369 | G | A | -0.802 |
| | ACATC | | | | | | |
| | (SEQ ID | | | | | | |
| | NO 19) | | | | | | |
| 2288850 | ACCA | ACCCCA | -2.814 | 4243325 | T | C | -0.030 |
| 2288850 | ACC | ACCC | -3.560 | 4243692 | G | A | -0.766 |
| 2288851 | CCAC | CCACAC | -3.736 | 4245311 | C | G | -2.320 |
| 2288852 | CACATC | CC | -4.433 | 4245850 | C | T | -0.411 |
| 2288852 | CACATC | CA | -4.176 | 4246833 | A | G | -4.315 |
| | GACCTC | | | | | | |
| | ATCGA | | | | | | |
| | (SEQ ID | | | | | | |
| | NO 20) | | | | | | |
| 2288859 | ACC | AA | -2.621 | 4247429 | A | G | -0.846 |
| 2288860 | C | A | -0.220 | 4247431 | G | A | -0.343 |
| 2288864 | A | T | 0.059 | 4247469 | A | C | -0.800 |
| 2288868 | A | C | -0.543 | 4247763 | T | C | -3.532 |
| 2288885 | C | T | -0.487 | 4247862 | T | G | -0.138 |
| 2288886 | C | T | -1.533 | 4248003 | A | G | -0.489 |

TABLE E
Streptomycin weighted positions; threshold value: −2.501

| POS | RN | SN | W | POS | RN | SN | W | POS | RN | SN | W |
|---|---|---|---|---|---|---|---|---|---|---|---|
| 107937 | ACC | ACCC | −3.307 | 2412085 | A | C | 0.03125 | 4407931 | A | G | −2.556 |
| 1084899 | C | T | −1.268 | 2621640 | G | A | −0.3229 | 4407940 | A | G | −4.711 |
| 1131738 | T | C | −3.258 | 2714460 | C | T | −3.597 | 4407949 | T | G | −2.784 |
| 1132890 | C | T | −0.333 | 2715016 | C | T | 0.5003 | 4407955 | C | G | −1.882 |
| 1237557 | A | C | −3.307 | 2752168 | G | T | −2.701 | 4407979 | G | C | −4.377 |
| 1238164 | A | C | −0.222 | 2876174 | G | A | −0.2572 | 4407982 | A | T | −1.934 |
| 1471861 | T | A | −1.934 | 2937665 | C | A | 2.861 | 4407994 | C | T | −4.925 |
| 1472015 | A | G | −1.400 | 2939373 | G | C | 0.1764 | 4407997 | C | T | −3.444 |
| 1472069 | G | T | −3.307 | 2939609 | C | A | −4.335 | 4408009 | ACC | ACCC | −2.98 |
| 1472102 | G | A | −0.465 | 2939891 | G | A | −0.8905 | 4408048 | C | A | −1.669 |
| 1472127 | CGG | TAC | 4.475 | 2955145 | G | A | −0.6694 | 4408059 | A | T | −4.94 |
| 1472143 | GA | GTA | −3.112 | 2955957 | A | C | −0.1259 | 4408061 | GCC | GC | −4.197 |
| 1472359 | A | C | −5.201 | 2956571 | G | C | −3.307 | 4408064 | G | A | −2.236 |
| 1472359 | A | T | −5.186 | 3074103 | G | A | −5.016 | 4408069 | C | T | −4.536 |
| 1472362 | C | T | −4.232 | 3074246 | CGTGCT | CGTGCT | −0.6583 | 4408087 | CGG | CG | −4.297 |
| | | | | | CGTGC | CGTGGT | | | | | |
| | | | | | (SEQ ID | GCTCGT | | | | | |
| | | | | | NO 021) | GC (SEQ | | | | | |
| | | | | | | ID NO | | | | | |
| | | | | | | 022) | | | | | |
| 1472418 | T | C | 0.688 | 3272997 | A | G | −2.493 | 4408100 | GCCCC | GCCCC | −4.933 |
| | | | | | | | | | CG | G | |
| 1472548 | A | G | −4.951 | 3296639 | ACC | AC | −1.609 | 4408102 | C | A | −3.7 |
| 1472723 | G | A | 0.607 | 3296792 | G | C | −1.488 | 4408115 | CGG | CG | −5.634 |
| 1472750 | C | A | −0.979 | 3296843 | A | G | 1.422 | 4408161 | CGG | CG | −3.621 |
| 1472751 | A | G | −4.137 | 3296962 | G | C | −4.921 | 491028 | C | T | 2.099 |
| 1472753 | A | C | −0.462 | 3297704 | G | A | −0.1068 | 511170 | C | G | −0.7074 |
| 1473170 | A | C | −1.642 | 3326811 | C | G | −0.06629 | 513282 | G | A | −3.307 |
| 1473246 | A | G | −0.182 | 3388838 | G | A | 2.67 | 513687 | C | T | −1.763 |
| 1473254 | A | C | −3.128 | 346309 | C | G | −2.229 | 514306 | C | A | −0.3984 |
| 1473870 | G | A | −0.068 | 346399 | T | C | −2.632 | 514357 | T | A | 4.212 |
| 1474007 | A | C | −0.652 | 3470150 | G | A | 0.4771 | 514657 | T | G | 0.00495 |
| 1474277 | C | A | −0.642 | 347449 | C | G | −3.307 | 514684 | C | T | 1.008 |
| 1474298 | T | G | 0.975 | 347650 | C | T | −0.08985 | 514911 | T | G | −1.425 |
| 1474527 | T | G | −4.921 | 3514512 | G | C | 0.1345 | 515190 | G | A | 0.5394 |
| 1474740 | G | T | −0.801 | 355677 | G | T | −0.9013 | 5548 | A | G | −3.464 |
| 1474852 | T | C | −1.227 | 355745 | C | T | −0.6918 | 5722 | G | T | −4.92 |
| 1475343 | A | G | −4.800 | 3570207 | G | A | 0.5976 | 575503 | G | A | −3.656 |
| 1475479 | C | G | 0.671 | 3723383 | G | T | −5.091 | 575562 | T | C | −2.541 |
| 1475510 | A | G | −4.920 | 3836358 | G | C | 6.269 | 576057 | TGGT | TGGGGT | −1.426 |
| 1475778 | G | A | −5.092 | 403565 | T | G | 0.1425 | 576372 | C | G | −1.435 |
| 1476016 | T | C | −4.920 | 404132 | C | T | −0.1892 | 576526 | C | T | −1.416 |
| 1476056 | G | A | −0.066 | 404356 | T | G | 9.469 | 576564 | T | G | 3.904 |
| 1476105 | G | A | 3.738 | 4238675 | C | T | −0.1677 | 576660 | T | G | −3.307 |
| 1476704 | GCCCC | GCCCC | −0.041 | 4239965 | G | A | −4.92 | 576710 | C | G | −0.1274 |
| | CCG | CG | | | | | | | | | |
| 1502120 | C | A | 0.133 | 4239976 | G | A | −0.06628 | 576766 | G | A | −0.5185 |
| 1673604 | G | A | −0.692 | 4240562 | A | C | −0.2974 | 6016 | C | G | −2.956 |
| 1673949 | G | A | 1.503 | 4241281 | G | A | −2.686 | 60875 | G | A | −2.044 |
| 1674263 | T | C | −0.545 | 4241292 | T | G | 8.077 | 6122 | G | A | −5.066 |
| 1674481 | T | G | −1.032 | 4242312 | G | A | 0.6754 | 614975 | G | C | −0.1796 |
| 1674782 | T | C | −0.141 | 4242595 | C | A | −4.951 | 615752 | C | G | 0.1415 |
| 1674981 | C | T | 0.806 | 4243667 | C | G | −0.7131 | 615791 | G | C | 4.246 |
| 1719877 | A | G | −2.059 | 4243969 | T | C | −1.474 | 616092 | G | T | −0.01828 |
| 1759422 | A | G | 1.151 | 4244069 | C | T | 2.897 | 6302 | G | T | −4.017 |
| 1799713 | A | C | 0.299 | 4244832 | G | A | −0.3032 | 648774 | C | T | −1.578 |
| 1800177 | G | A | −2.087 | 4245075 | G | C | −2.681 | 649235 | C | G | −1.066 |
| 1817289 | C | T | 0.359 | 4246869 | G | A | 1.008 | 6794 | A | G | −4.769 |
| 1833594 | C | A | 3.001 | 4246941 | C | T | −1.486 | 6878 | T | C | −0.2723 |
| 1834953 | C | G | 1.000 | 4247070 | T | C | −3.258 | 6937 | G | T | −4 |
| 1849051 | C | T | −0.067 | 4247429 | A | G | −0.6171 | 6989 | G | C | 1.774 |
| 1875556 | G | C | −0.154 | 4247431 | G | C | −0.07703 | 7219 | C | T | −0.06628 |
| 1875562 | G | C | −0.172 | 4247513 | T | C | −0.6503 | 7362 | G | C | 0.041 |
| 1875587 | GGT | CGG | −1.780 | 4247574 | A | C | −0.05156 | 7510 | A | G | −2.287 |
| 1875754 | A | C | −1.859 | 4247999 | G | A | 2.769 | 7570 | C | T | −0.3531 |
| 1877231 | TGGGT | GGGGG | −1.354 | 4248003 | A | G | −0.03969 | 7570 | C | G | −0.3752 |
| 1877744 | A | C | −0.056 | 4248758 | C | T | −0.8352 | 7581 | GACAG | TGCAC | −1.934 |
| 1877918 | T | G | −1.669 | 4249007 | C | T | −4.2 | 7583 | CAG | CAC | 4.559 |
| 1879244 | T | C | −0.666 | 4249327 | G | A | −4.343 | 760404 | C | T | 8.16 |
| 1879316 | C | T | −5.066 | 4257095 | A | C | −1.978 | 760480 | G | C | −0.08093 |
| 1879742 | C | G | −2.340 | 4258223 | T | G | −1.033 | 760640 | C | T | −2.574 |
| 1881059 | C | T | −4.503 | 4258955 | T | G | 0.02962 | 761110 | A | T | −0.09844 |
| 1881536 | A | C | −0.643 | 4259418 | A | G | −0.06712 | 761114 | GAACAA | GAACC | −0.1462 |
| | | | | | | | | | CC | | |
| 1881540 | A | C | −0.011 | 4259507 | A | C | −0.3503 | 761127 | TC | CA | 3.026 |
| 1882475 | G | A | −2.112 | 4269606 | A | G | 0.09195 | 761139 | C | T | −0.1047 |
| 1882831 | T | C | 0.314 | 4308852 | G | T | −3.22 | 761139 | C | G | −0.4019 |
| 1883039 | A | C | 0.108 | 4326135 | G | T | 4.777 | 761155 | C | G | −0.6907 |
| 1883378 | A | G | −0.415 | 4326279 | ACACC | ACACCA | 0.4032 | 761155 | C | T | −0.7283 |
| | | | | | | CC | | | | | |
| 1883766 | T | G | −0.188 | 4326391 | C | T | −5.073 | 761161 | T | C | −0.01422 |
| 1883768 | A | G | −0.253 | 4326620 | T | G | 3.444 | 761277 | A | T | −1.57 |
| 1884469 | T | G | −0.338 | 4326695 | A | T | −3.573 | 761382 | A | T | −1.814 |
| 1885385 | T | G | −0.074 | 4326842 | T | G | −0.009964 | 7615 | T | G | −3.307 |
| 1885562 | T | C | −1.374 | 4326864 | T | C | −3.778 | 761889 | G | C | −2.882 |
| 1885835 | A | T | 0.829 | 4326917 | G | A | −2.418 | 762089 | G | C | −0.1007 |
| 2054069 | G | T | −4.701 | 4327067 | A | C | −2.745 | 762179 | C | T | −0.5709 |
| 2101769 | C | A | −2.318 | 4327229 | A | G | 0.07244 | 763155 | G | C | 1.869 |
| 2102207 | C | T | −0.652 | 4327301 | T | G | −0.7666 | 763277 | A | T | −2.637 |
| 2134064 | G | A | −1.424 | 4327333 | CAA | CA | −2.986 | 764617 | C | T | 1.189 |
| 2154461 | CG | CTG | −0.830 | 4399385 | C | T | −0.0238 | 766645 | A | C | −0.1773 |
| 2154486 | G | T | 3.833 | 4399794 | G | A | −0.6918 | 766978 | C | T | 1.454 |
| 2154695 | C | G | 1.362 | 4403351 | G | A | 0.001327 | 7705 | C | G | −0.03499 |
| 2154739 | A | T | −4.387 | 4403900 | A | G | 0.1405 | 7776 | C | G | −3.004 |
| 2154746 | CAA | CA | −1.883 | 4407596 | C | G | −4.769 | 779279 | C | T | −0.8469 |
| 2154834 | G | C | −0.237 | 4407686 | C | A | −4.8 | 781687 | A | G | −5.212 |
| 2155163 | T | C | −2.707 | 4407713 | CTT | CT | −5.356 | 781768 | A | C | −3.258 |
| 2155167 | GC | TG | −0.547 | 4407732 | C | T | 0.9338 | 781822 | A | G | −5.343 |
| 2155168 | C | G | −1.689 | 4407748 | A | G | 0.1723 | 781822 | A | T | −3.838 |
| 2155168 | C | T | −0.557 | 4407790 | G | A | −4.6 | 781822 | A | C | −2.717 |
| 2155648 | T | C | −0.031 | 4407790 | G | T | −4.221 | 7879 | A | G | −0.6029 |
| 2155746 | G | A | 2.732 | 4407796 | G | T | −1.879 | 7914 | C | A | −0.07119 |
| 2155801 | C | T | −0.023 | 4407796 | G | C | −3.177 | 796747 | T | G | −1.174 |
| 2155968 | C | T | −2.366 | 4407802 | G | A | −4.951 | 798355 | G | C | −0.06799 |
| 2223293 | T | C | −1.426 | 4407807 | G | A | −3.712 | 8056 | G | T | −0.9141 |
| 2288730 | G | T | 5.051 | 4407816 | GCC | GC | −5.878 | 8554 | G | A | 0.2409 |
| 2288785 | TGG | TGGG | −0.548 | 4407830 | G | A | −4.672 | 8646 | C | G | −4.751 |
| 2288953 | C | G | 2.937 | 4407846 | GGCGC | GGCGC | −4.956 | 875532 | C | G | −3.307 |
| | | | | | GCC | C | | | | | |
| 2289031 | G | A | −3.904 | 4407850 | C | A | −4.885 | 875772 | G | A | −0.2079 |
| 2289038 | C | A | −3.956 | 4407851 | GCCCC | GCCCC | −4.1 | 8806 | A | G | 0.2318 |
| | | | | | CG | G | | | | | |
| 2289068 | G | C | 0.272 | 4407851 | G | T | −2.117 | 891788 | C | G | −2.299 |
| 2289140 | G | C | 0.009 | 4407851 | GCCCC | GCCCC | −2.275 | 9276 | C | T | −0.5072 |
| | | | | | CG | CCG | | | | | |
| 2289144 | T | A | −2.713 | 4407876 | GCC | GC | −5.199 | 9437 | G | A | −0.5645 |
| 2410821 | C | T | −0.097 | 4407925 | G | A | −3.32 | 9724 | A | T | −2.681 |
| 9803 | C | T | −0.032 | | | | | | | | |

TABLE F
Ciprofloxacin weighted positions; threshold value: −4.815

| POS | RN | SN | W | POS | RN | SN | W | POS | RN | SN | W |
|---|---|---|---|---|---|---|---|---|---|---|---|
| 1472621 | G | A | −0.416 | 3326232 | G | C | −0.7143 | 7582 | ACAG | GCAC | −3.27 |
| 1476442 | T | C | −0.611 | 347221 | G | C | −1.797 | 7585 | G | C | 0.1673 |
| 1879388 | C | T | −0.523 | 4247431 | G | C | −0.4812 | 760493 | C | A | −0.6532 |
| 2154695 | C | G | −0.017 | 4247431 | G | A | −0.318 | 761277 | A | T | −1.293 |
| 2288771 | CA | CTA | −5.862 | 4249403 | A | C | −0.2356 | 761293 | T | C | −0.4454 |
| 2289052 | A | C | −0.476 | 4326231 | CAA | CA | −0.7394 | 766070 | C | T | −0.6943 |
| 2620781 | T | C | −0.506 | 4408100 | GCCCC | GCCCC | −0.3498 | 7960 | T | G | −5.49 |
| | | | | | CG | G | | | | | |
| 2937779 | C | T | −0.267 | 7521 | G | T | −5.199 | 875309 | T | G | −5.599 |
| 9224 | C | G | −2.581 | | | | | | | | |

TABLE G
Moxifloxacin weighted positions; threshold value: −2.547

| POS | RN | SN | W | POS | RN | SN | W | POS | RN | SN | W |
|---|---|---|---|---|---|---|---|---|---|---|---|
| 1084993 | G | C | −0.011 | 2288771 | CA | CTA | −1.488 | 4261145 | A | C | −0.3533 |
| 1473835 | G | A | −0.399 | 2726339 | GGT | GGG | −0.1374 | 4308881 | C | T | −0.1004 |
| 1759252 | G | T | −1.000 | 3569508 | G | A | −0.3697 | 4326045 | CCTC | CCTCTC | −0.5565 |
| 1834836 | T | C | −0.248 | 403406 | A | C | −0.4319 | 4326333 | C | G | −0.3491 |
| 1875869 | T | G | −0.129 | 4241022 | C | T | −1.488 | 4399185 | C | T | −0.3303 |
| 1877968 | C | G | −0.148 | 4242369 | G | C | −0.3386 | 4399629 | C | G | −0.2734 |
| 1884026 | C | T | −0.224 | 4247249 | G | C | −0.288 | 497254 | T | C | −0.7099 |
| 2053682 | C | T | −0.002 | 4247730 | G | A | −0.3273 | 538621 | C | G | −0.3397 |
| 2053759 | C | T | −0.271 | 4249401 | T | G | −0.4664 | 541262 | A | G | −0.1039 |
| 2154695 | C | G | −0.506 | 4249664 | A | C | −0.2223 | 7582 | ACAG | GCAC | −0.09353 |
| 2155168 | C | G | −0.165 | 4259744 | G | C | −0.2165 | 761100 | C | A | −0.527 |
| 779369 | A | C | −0.984 | | | | | | | | |
| 801174 | T | C | −0.397 | | | | | | | | |

TABLE H
Ofloxacin weighted positions; threshold value: −1.774

| POS | RN | SN | W | POS | RN | SN | W | POS | RN | SN | W |
|---|---|---|---|---|---|---|---|---|---|---|---|
| 1085605 | C | T | −2.724 | 2289156 | CGG | CGGG | −0.001581 | 538656 | G | T | −0.5569 |
| 1472058 | T | G | −0.406 | 2289163 | GCGCG | GC | −0.4793 | 538681 | C | A | −1.203 |
| | | | | | GCGCC | | | | | | |
| | | | | | ACC | | | | | | |
| | | | | | (SEQ ID | | | | | | |
| | | | | | NO 014) | | | | | | |
| 1472069 | G | T | −2.724 | 2289223 | C | G | 2.927 | 5555 | G | A | −4.358 |
| 1472155 | C | A | −2.707 | 2289226 | T | G | 0.1411 | 615211 | T | G | 0.02529 |
| 1472723 | G | A | −0.221 | 2519280 | C | T | 1.793 | 62904 | G | A | −0.02509 |
| 1472920 | GTT | GTTT | −0.553 | 2714885 | GAA | GA | 4.618 | 648774 | C | T | −2.261 |
| 1473012 | G | A | −5.674 | 2726636 | G | A | −0.1754 | 6620 | G | A | −5.415 |
| 1473246 | A | G | −0.656 | 2751804 | C | T | 1.26 | 6620 | G | C | −4.534 |
| 1473254 | A | C | −2.080 | 2751978 | G | C | −0.4759 | 6734 | A | T | −5.343 |
| 1473344 | T | C | 9.306 | 2939657 | T | C | 0.1112 | 6734 | A | G | −0.05677 |
| 1475570 | AATGCC | TTTACC | −0.124 | 2956571 | G | C | −2.724 | 6735 | A | C | −0.05309 |
| | C | T | | | | | | | | | |
| 1487974 | ACCC | CCCG | −0.654 | 3074230 | T | C | −2.567 | 6735 | A | G | −5.747 |
| 1488006 | C | G | −1.610 | 3272997 | A | G | −0.3665 | 6736 | C | A | −4.197 |
| 1674709 | T | C | −2.677 | 3296639 | ACC | AC | −0.05368 | 6736 | C | G | −4.951 |
| 1674782 | T | C | −0.226 | 3296928 | CCA | CCC | −1.336 | 7457 | T | A | 1.966 |
| 1758588 | A | G | −0.218 | 3296956 | C | T | −6.045 | 7495 | GCC | GCCC | −2.724 |
| 1816017 | T | G | −0.620 | 3297018 | C | T | −1.367 | 7563 | G | T | −5.518 |
| 1833909 | A | C | −0.145 | 346275 | C | G | 0.6163 | 7564 | G | C | −6.123 |
| 1834177 | A | C | −0.014 | 3514392 | T | G | 7.944 | 7570 | C | T | −5.775 |
| 1875902 | T | C | −1.975 | 354822 | G | A | −0.6586 | 7570 | C | G | −3.652 |
| 1876603 | C | G | −2.953 | 355677 | G | T | −0.2409 | 7570 | CGT | TGC | −5.177 |
| 1879890 | C | T | −2.641 | 355709 | ACC | ACCC | 1.296 | 7572 | T | C | −6.169 |
| 1883716 | T | G | 0.018 | 3569277 | CTTG | CTTT | −0.5705 | 7581 | GACAG | AACAC | −5.086 |
| 1884817 | A | G | 2.649 | 3569325 | G | C | −1.213 | 7581 | GACAG | CACAC | −1.591 |
| 1886509 | T | G | −2.507 | 3569731 | T | G | −0.5217 | 7581 | GACAG | TACAC | −5.372 |
| 1917972 | A | G | 0.529 | 403358 | A | G | 1.702 | 7581 | GACAG | GACAC | 2.838 |
| 1918137 | AGCG | AGCGC | −0.318 | 404356 | T | G | 2.843 | 7581 | GACAG | TGCAC | −4.509 |
| | | G | | | | | | | | | |
| 2101853 | A | G | −1.533 | 4125409 | T | C | −0.5124 | 7581 | G | C | −3.872 |
| 2102492 | T | C | 2.261 | 4238092 | A | G | −1.487 | 7582 | ACAG | GCAC | −5.612 |
| 2133987 | C | T | −3.043 | 4247429 | A | G | −0.03336 | 7582 | ACAG | CCAC | −6.135 |
| 2153852 | T | G | −0.406 | 4248003 | A | G | −0.2313 | 7582 | ACAG | ACAC | 4.483 |
| 2154695 | C | A | −0.165 | 4257750 | CGG | CG | 3.084 | 7582 | ACAG | TCAC | −4.328 |
| 2154695 | C | G | 0.086 | 4269271 | A | G | −0.03665 | 7582 | A | C | −5.026 |
| 2155168 | C | G | −0.406 | 4307609 | T | G | −0.3574 | 7582 | A | G | 2.073 |
| 2155430 | T | G | −2.510 | 4307617 | A | C | −0.568 | 7583 | CAG | CAC | 3.761 |
| 2155563 | G | T | −0.516 | 4326083 | T | C | −0.01748 | 7585 | G | C | 0.8611 |
| 2155648 | T | C | −0.428 | 4326087 | GTA | GA | −0.4221 | 760978 | A | G | −0.322 |
| 2155687 | T | C | −5.483 | 4326251 | TAATTC | TAATTC | −6.805 | 761110 | A | G | −0.5045 |
| | | | | | A | AATTCA | | | | | |
| | | | | | | (SEQ ID | | | | | |
| | | | | | | NO 023) | | | | | |
| 2223293 | T | C | −0.314 | 4326282 | C | A | −2.724 | 761131 | G | A | −1.16 |
| 2223448 | G | A | 3.028 | 4326625 | GCC | GC | 8.419 | 761155 | C | T | −0.287 |
| 2288805 | G | T | −0.476 | 4326801 | GCT | GCCT | −0.8729 | 761292 | G | T | 5.647 |
| 2288835 | T | C | 8.082 | 4326879 | C | T | 8.546 | 7615 | T | G | −4.844 |
| 2288852 | CACATC | CC | −1.144 | 4327333 | CAA | CA | 2.408 | 762399 | G | T | −3.589 |
| 2288852 | CACATC | CA | −0.445 | 4399261 | GCA | GCC | −4.209 | 763123 | T | C | −0.6369 |
| | GACCTC | | | | | | | | | | |
| | ATCGA | | | | | | | | | | |
| | (SEQ | | | | | | | | | | |
| | ID NO | | | | | | | | | | |
| | 020) | | | | | | | | | | |
| 2288919 | C | T | −4.002 | 4407781 | G | T | −0.2273 | 763320 | G | C | −5.301 |
| 2288942 | GGTG | GG | 9.363 | 4407927 | T | G | −0.01711 | 764617 | C | T | 1.998 |
| 2288988 | A | C | 2.125 | 4408100 | GCCCC | GCCCC | −0.7626 | 7776 | C | G | −2.77 |
| | | | | | CG | G | | | | | |
| 2289039 | C | A | −0.040 | 490751 | T | G | −0.4575 | 779350 | G | A | 0.795 |
| 2289049 | AA | ACA | 1.874 | 514357 | T | A | −2.312 | 784125 | G | C | −1.664 |
| 2289096 | T | C | −0.242 | 514390 | A | C | 9.738 | 796747 | T | G | −0.2752 |
| 798355 | G | C | −0.034 | | | | | | | | |
| 875532 | C | G | −2.724 | | | | | | | | |

TABLE I
Amikacin weighted positions; threshold value: −4.729

| POS | RN | SN | W | POS | RN | SN | W | POS | RN | SN | W |
|---|---|---|---|---|---|---|---|---|---|---|---|
| 1136668 | G | T | −1.482 | 1879849 | GC | GG | −2.401 | 3296925 | GCACCA | CCCCCC | −4.413 |
| | | | | | | | | | | GCCCCA | |
| | | | | | | | | | | (SEQ ID | |
| | | | | | | | | | | NO 030) | |
| 1473246 | A | G | −6.101 | 1880429 | T | G | −0.2512 | 3296932 | C | A | −2.458 |
| 1473247 | C | T | −3.235 | 1881922 | C | G | −0.3283 | 3570220 | T | C | −0.1187 |
| 1475703 | ACGAT | GCGAA | −0.824 | 1884305 | T | G | −0.1133 | 3723401 | G | A | −0.1123 |
| 1476212 | T | G | −0.638 | 2155168 | C | G | −0.1161 | 4237971 | A | T | −0.5922 |
| 1719692 | A | C | −0.529 | 2155687 | T | C | −5.581 | 4238675 | C | T | −0.2387 |
| 1850310 | G | C | −0.104 | 2289111 | A | C | −4.197 | 4399236 | GGAGG | GGGGG | 1.623 |
| | | | | | | | | | CC | CGGGA | |
| | | | | | | | | | | GGCG | |
| | | | | | | | | | | (SEQ ID | |
| | | | | | | | | | | NO 024) | |
| 1875556 | G | C | −0.167 | 2726338 | T | G | −0.1279 | 4399253 | GC | GG | −4.429 |
| 1875562 | G | C | −0.088 | 2726339 | GGT | GGG | −2.483 | 614975 | G | C | −0.1446 |
| 1875577 | T | G | −0.982 | 2939372 | AG | GC | −0.02616 | 6232 | C | A | −0.6646 |
| 1877866 | G | C | 1.707 | 2940714 | A | C | −0.2417 | 761135 | GACCCA | TACCTG | −2.419 |
| 1879685 | T | G | −1.360 | 3272659 | T | A | −1.407 | 761535 | C | G | −1.545 |
| 766645 | A | C | −0.180 | | | | | | | | |
| 781687 | A | G | −0.113 | | | | | | | | |

TABLE J
Capreomycin weighted positions; threshold value: −1.531

| POS | RN | SN | W | POS | RN | SN | W |
|---|---|---|---|---|---|---|---|
| 1472172 | T | C | 0.118 | 2155648 | T | C | −0.223 |
| 1472337 | C | T | 0.288 | 2155687 | T | C | −1.184 |
| 1472362 | C | T | −0.106 | 2288850 | ACCA | ACCCCA | 0.456 |
| 1472753 | A | C | 0.057 | 2288953 | C | T | −0.061 |
| 1472793 | A | T | 0.132 | 2289228 | A | G | −0.442 |
| 1473246 | A | G | −0.887 | 2519176 | G | A | 0.102 |
| 1474324 | C | T | −0.900 | 2621058 | G | A | 0.012 |
| 1476481 | T | C | 0.032 | 2714109 | G | A | −0.033 |
| 156736 | C | T | −1.264 | 2714846 | C | T | 0.014 |
| 1674162 | C | T | 0.187 | 2726338 | T | G | −0.079 |
| 1674481 | T | G | 0.135 | 2726339 | GGT | GGG | −0.073 |
| 1757844 | T | G | 0.006 | 2874707 | C | G | −0.341 |
| 1758180 | T | G | −0.263 | 2876062 | CCT | CCC | −0.068 |
| 1759252 | G | T | −1.302 | 2939017 | T | G | −0.204 |
| 1816011 | T | G | −0.065 | 2956497 | A | C | −1.092 |
| 1816983 | C | T | −0.128 | 3073868 | T | C | 0.007 |
| 1848682 | G | T | −0.108 | 3273102 | C | T | −0.160 |
| 1851759 | T | G | −0.297 | 3296843 | A | G | 0.030 |
| 1875423 | GGGG | CGGA | −0.793 | 3465987 | G | A | −0.859 |
| 1875528 | C | G | −0.320 | 3466525 | T | G | 0.102 |
| 1876202 | T | G | 0.064 | 347007 | C | G | 0.019 |
| 1876332 | GGGC | CGGG | −0.187 | 354606 | C | T | −0.909 |
| 1877652 | G | A | −0.329 | 3835465 | T | A | −1.300 |
| 1878562 | T | G | −0.378 | 3977610 | C | G | 0.052 |
| 1879841 | T | G | −0.060 | 4125398 | T | G | −0.102 |
| 1880574 | T | C | −0.167 | 4229168 | GA | GC | −0.207 |
| 1883750 | T | G | −0.206 | 4238632 | T | G | −0.957 |
| 1885872 | T | C | 0.041 | 4238972 | C | G | 0.027 |
| 1885888 | AATA | GATG | −0.096 | 4239476 | G | T | −0.290 |
| 1917851 | C | A | −0.037 | 4240153 | G | A | −0.827 |
| 1918230 | C | A | −0.772 | 4243963 | T | G | −0.110 |
| 2101850 | A | C | −1.424 | 4249408 | G | A | 0.066 |
| 2153807 | A | C | −0.004 | 4249583 | G | A | 0.011 |
| 2155168 | C | G | −0.028 | 4256011 | G | A | −0.243 |
| 2155541 | A | G | −0.085 | 4257220 | A | G | 0.120 |

TABLE K
Kanamycin weighted positions; threshold value: −3.033

| POS | RN | SN | W | POS | RN | SN | W | POS | RN | SN | W |
|---|---|---|---|---|---|---|---|---|---|---|---|
| 107937 | ACC | ACCC | −3.632 | 2288805 | G | T | −5.032 | 4326855 | G | C | −0.04238 |
| 108113 | T | G | 1.700 | 2288824 | GCACAC | GC | −0.7879 | 4326943 | G | C | 0.03236 |
| | | | | | AATGAT | | | | | | |
| | | | | | C (SEQ | | | | | | |
| | | | | | ID NO | | | | | | |
| | | | | | 025) | | | | | | |
| 1084174 | C | T | −1.002 | 2288835 | T | C | −1.406 | 4327132 | GTT | GT | −1.07 |
| 1084280 | A | G | 0.627 | 2288839 | T | G | −0.2695 | 4327323 | G | A | −0.09909 |
| 1084814 | A | C | −0.835 | 2288883 | A | G | −4.01 | 4328212 | CGGGG | CGGGG | −0.8349 |
| | | | | | | | | | GT | T | |
| 1084836 | T | G | −0.785 | 2288938 | C | T | −3.261 | 4398852 | C | T | −2.862 |
| 1472069 | G | T | −3.632 | 2288942 | GGTG | GG | 3.59 | 4399683 | C | G | −0.3013 |
| 1472621 | G | A | −1.451 | 2288956 | T | C | −0.4191 | 4403597 | G | A | −3.134 |
| 1472920 | GTT | GTTT | −4.694 | 2289040 | A | C | 0.8196 | 4407927 | T | G | −0.05477 |
| 1473131 | G | A | 2.843 | 2289052 | A | C | −1.59 | 495040 | G | A | −2.853 |
| 1473246 | A | G | −5.884 | 2289058 | G | A | −0.6329 | 497491 | G | A | −0.1604 |
| 1473247 | C | T | −5.236 | 2289081 | G | T | −0.8971 | 514357 | T | A | 3.147 |
| 1473329 | G | T | −2.156 | 2289090 | T | C | −4.77 | 540097 | T | A | −0.9936 |
| 1474234 | G | A | −0.920 | 2289092 | GAA | GA | −3.982 | 540587 | G | A | −3.982 |
| 1474584 | C | G | 2.733 | 2289100 | T | C | −3.779 | 541262 | A | G | −3.39 |
| 1474833 | G | T | −2.644 | 2289103 | T | C | −0.5849 | 575688 | CGG | CG | −4.189 |
| 1475816 | CA | GG | 0.079 | 2289119 | G | C | 4.153 | 575834 | T | C | −0.6272 |
| 1491272 | G | A | 0.069 | 2289218 | G | T | 1.856 | 576031 | TGG | TG | −4.045 |
| 1501301 | C | T | −8.326 | 2289254 | C | A | 1.014 | 576273 | CGG | CGACG | −3.978 |
| | | | | | | | | | | GACTGG | |
| | | | | | | | | | | TCTGGC | |
| | | | | | | | | | | TTCACC | |
| | | | | | | | | | | G (SEQ | |
| | | | | | | | | | | ID NO | |
| | | | | | | | | | | 026) | |
| 1502561 | C | A | −2.865 | 2505553 | A | G | −4.87 | 5849 | C | A | 1.82 |
| 1502745 | A | G | −2.641 | 2620781 | T | C | −0.7788 | 60373 | T | A | 0.5306 |
| 1674417 | C | T | 0.560 | 2693846 | T | G | 0.3064 | 61178 | C | T | −5.453 |
| 1674837 | C | T | −5.246 | 2694792 | G | A | 0.3661 | 648774 | C | T | −3.265 |
| 1758588 | A | G | −0.426 | 2694909 | G | A | −4.279 | 6579 | C | T | −0.1187 |
| 1817357 | T | G | −2.800 | 2714130 | C | G | −1.068 | 6817 | G | A | −0.1101 |
| 1850557 | A | G | −0.414 | 2714610 | GAG | GAAG | −4.01 | 7495 | GCC | GCCC | −3.474 |
| 1875439 | T | G | −1.992 | 2715319 | AG | ACG | −2.783 | 7570 | C | T | −0.5245 |
| 1875509 | A | C | −2.645 | 2751804 | C | T | 0.4874 | 7572 | T | C | −0.4159 |
| 1875902 | T | C | −3.628 | 2752085 | GC | AA | −2.28 | 7581 | GACAG | GACAC | −4.01 |
| 1876559 | C | T | 1.482 | 2939816 | T | C | 1.07 | 7582 | ACAG | CCAC | −0.005512 |
| 1876886 | T | G | −2.228 | 3296753 | G | C | −5.453 | 7583 | CAG | CAC | 0.9808 |
| 1877624 | G | C | −3.531 | 3296848 | G | A | −4.509 | 7584 | AG | GC | −0.2459 |
| 1878416 | A | C | −3.552 | 3388522 | C | T | −2.956 | 760461 | C | T | −3.324 |
| 1879682 | T | C | −2.861 | 346275 | C | G | 0.9186 | 761110 | A | G | −0.3047 |
| 1879713 | G | T | −5.136 | 349054 | C | T | −2.553 | 761155 | C | T | −0.2511 |
| 1879878 | TGC | GGG | 0.777 | 349331 | G | A | −4.415 | 761161 | T | C | −0.1584 |
| 1882933 | T | A | −3.552 | 354679 | C | T | −0.3462 | 761225 | C | T | 2.469 |
| 1883179 | G | A | −3.758 | 355846 | T | C | 3.557 | 761268 | A | G | −0.5828 |
| 1884110 | C | G | 1.839 | 3835223 | C | T | −1.736 | 761314 | T | C | −0.7695 |
| 1884260 | G | A | 0.113 | 3986931 | C | A | −1.854 | 762285 | C | T | −0.6924 |
| 1885341 | A | G | −3.776 | 403448 | C | T | −2.38 | 763277 | A | T | −4.307 |
| 1885385 | T | G | −0.003 | 403565 | T | G | 0.06394 | 763438 | G | T | −2.228 |
| 1886275 | T | C | −1.264 | 403980 | G | A | 0.4496 | 763571 | GTGCG | ATGAAG | 0.6689 |
| | | | | | | | | | CTT | TA | |
| 1917991 | CGA | CGGA | 2.091 | 404130 | T | C | −0.2474 | 764674 | G | T | 1.266 |
| 1918029 | C | A | −1.109 | 405812 | T | C | −0.8692 | 764703 | A | G | −3.304 |
| 2101853 | A | G | −3.226 | 4124906 | A | C | 0.02798 | 764724 | T | C | −3.802 |
| 2101965 | C | T | 0.451 | 4125386 | C | G | −3.082 | 764822 | G | T | −0.2686 |
| 2102417 | G | A | −4.203 | 4125421 | A | G | −2.829 | 764841 | T | C | −1.032 |
| 2102912 | G | C | −3.457 | 4243346 | A | G | −0.9241 | 764918 | G | T | −0.1229 |
| 2153852 | T | G | 2.929 | 4246734 | T | G | −0.1134 | 764925 | G | A | −1.744 |
| 2154239 | T | C | −0.256 | 4246833 | A | G | −5.453 | 764931 | C | T | −3.261 |
| 2154461 | CG | CTG | −5.135 | 4247429 | A | G | −0.7102 | 764948 | T | G | 0.5548 |
| 2155168 | C | G | −0.211 | 4248254 | A | G | −4.585 | 781709 | G | T | 2.296 |
| 2155430 | T | G | −3.550 | 4257673 | G | A | 4.342 | 782750 | T | C | −5.453 |
| 2155648 | T | G | 0.505 | 4269298 | A | G | −0.5288 | 783404 | A | C | −3.532 |
| 2288703 | A | C | −1.328 | 4308395 | G | A | −0.1075 | 784557 | GAA | GAAA | 2.354 |
| 2288704 | C | G | −0.020 | 4326120 | GCG | GG | −2.678 | 784696 | C | T | 1.757 |
| 2288724 | TCC | TCCC | −0.660 | 4326279 | ACACC | ACACCA | −1.455 | 7930 | C | T | −0.5771 |
| | | | | | | CC | | | | | |
| 2288778 | ACCC | ACCCCC | −4.319 | 4326282 | C | A | −3.632 | 800940 | C | G | −1.702 |
| 2288782 | T | C | −0.152 | 4326705 | GCC | GC | −5.636 | 801019 | A | G | −4.512 |
| 2288785 | TGG | TGGG | −0.131 | 4326718 | GGCG | GGCGC | −0.4992 | 875532 | C | G | −3.632 |
| | | | | | | G | | | | | |
| 9065 | C | A | −3.010 | | | | | | | | |
| 931306 | G | C | −1.857 | | | | | | | | |

TABLE L
Prothionamide weighted positions; threshold value: −0.551

| POS | RN | SN | W | POS | RN | SN | W | POS | RN | SN | W |
|---|---|---|---|---|---|---|---|---|---|---|---|
| 3272251 | T | G | 0.102 | 4258428 | C | G | −0.1321 | 4307617 | A | C | 0.02583 |
| 783585 | G | A | −0.244 | | | | | | | | |

TABLE M
Ethionamide weighted positions; threshold value: −2.589

| POS | RN | SN | W | POS | RN | SN | W | POS | RN | SN | W |
|---|---|---|---|---|---|---|---|---|---|---|---|
| 107937 | ACC | ACCC | −2.881 | 2693848 | G | T | −0.7518 | 4326801 | GCT | GCCT | −4.616 |
| 1084335 | G | C | −0.710 | 2693851 | G | T | −0.5122 | 4326833 | GACA | GA | −4.65 |
| 1085028 | C | G | −1.238 | 2693853 | T | G | −0.9397 | 4326858 | G | A | −4.718 |
| 1085705 | G | C | −0.057 | 2693934 | T | C | −0.02244 | 4326964 | GTC | GC | −1.913 |
| 1128074 | A | G | −0.514 | 2694909 | G | A | −0.1503 | 4326994 | CGG | CG | −3.73 |
| 1132004 | A | C | −0.967 | 2714308 | G | A | −4.551 | 4327027 | C | A | −3.543 |
| 1133169 | T | G | −0.470 | 2726287 | G | A | −3.075 | 4327033 | G | C | −3.237 |
| 1138013 | G | A | −0.415 | 273099 | A | G | −1.703 | 4327091 | GCG | GCCG | −1.829 |
| 1237205 | A | C | −0.047 | 273449 | C | A | −1.049 | 4327132 | GTT | GT | −2.711 |
| 1461309 | C | T | −0.192 | 274030 | C | T | −1.186 | 4327192 | GATTCC | GATTCC | −3.934 |
| | | | | | | | | | A | ATTCCA | |
| | | | | | | | | | | (SEQ ID | |
| | | | | | | | | | | NO 027) | |
| 1472069 | G | T | −2.881 | 2832239 | CGG | CG | −1.572 | 4327231 | GA | GTA | −4.292 |
| 1472723 | G | A | −1.511 | 2873971 | A | C | −0.09585 | 4327267 | C | T | −4.309 |
| 1472753 | A | C | −2.195 | 2937134 | G | A | −1.76 | 4327269 | AGGG | AGGGG | −2.86 |
| | | | | | | | | | | G | |
| 1473123 | A | G | −1.550 | 2939020 | A | T | −2.825 | 4327294 | G | T | −3.525 |
| 1473246 | A | G | −0.121 | 2940719 | A | C | −0.009038 | 4327325 | TAA | TA | −3.936 |
| 1473254 | A | C | −2.184 | 2956379 | C | T | −1.751 | 4327333 | CAA | CA | −3.935 |
| 1473329 | G | T | −0.200 | 3073979 | G | A | −1.953 | 4327334 | AAATCC | AC | −0.08139 |
| 1475395 | T | C | −3.269 | 3074246 | CGTGCT | CGTGCT | −4.03 | 4327340 | C | T | −1.155 |
| | | | | | CGTGC | CGTGGT | | | | | |
| | | | | | (SEQ ID | GCTCGT | | | | | |
| | | | | | NO 021) | GC (SEQ | | | | | |
| | | | | | | ID NO | | | | | |
| | | | | | | 022) | | | | | |
| 1475778 | G | A | −1.561 | 3272273 | C | G | −0.1343 | 4327363 | CTT | CT | −3.845 |
| 1476114 | T | C | −4.177 | 3272700 | T | G | −0.06887 | 4327404 | G | A | −4.291 |
| 1476382 | A | G | −0.643 | 3296115 | A | C | −0.5959 | 4327441 | GCC | GC | −3.223 |
| 1500988 | C | T | −0.554 | 3296271 | T | C | −3.287 | 4328212 | CGGGG | CGGGG | −2.268 |
| | | | | | | | | | GT | T | |
| 1502561 | C | A | −1.751 | 3296843 | A | G | 0.5174 | 4399181 | T | C | −0.1061 |
| 1502745 | A | G | −1.559 | 3296874 | T | G | −0.2851 | 4399683 | C | G | −0.8073 |
| 1674356 | A | G | −3.591 | 3296886 | G | C | −0.08439 | 4407790 | G | T | −1.061 |
| 1718779 | A | C | −0.397 | 3296928 | CCACCG | CC | −0.7024 | 4407794 | G | A | −1.792 |
| | | | | | CCAGC | | | | | | |
| | | | | | (SEQ ID | | | | | | |
| | | | | | NO 003) | | | | | | |
| 1758384 | G | A | −1.091 | 3388522 | C | T | −1.765 | 4407798 | C | A | −0.2267 |
| 1758596 | T | C | −0.601 | 345504 | A | C | −1.399 | 4408061 | G | T | −0.503 |
| 1816078 | A | G | −0.038 | 346045 | G | C | −0.9491 | 4408174 | G | A | −1.273 |
| 1816098 | T | G | −0.125 | 346205 | C | T | −0.6064 | 495040 | G | A | −1.57 |
| 1816122 | T | G | −0.103 | 346563 | C | T | −3.252 | 511087 | A | C | −0.1636 |
| 1816983 | C | T | −1.441 | 347256 | C | T | −2.901 | 511546 | G | A | −0.9667 |
| 1833594 | C | A | −3.251 | 347393 | C | T | −3.288 | 512294 | T | G | −0.1516 |
| 1833626 | A | G | −4.061 | 349071 | A | G | −0.9593 | 512344 | C | G | −0.8387 |
| 1833909 | A | C | −1.504 | 349205 | A | C | −1.548 | 512610 | C | T | −0.5648 |
| 1833909 | A | G | −3.473 | 354535 | T | C | −0.2019 | 514344 | G | A | −1.536 |
| 1875499 | G | T | −3.269 | 354586 | T | C | −4.74 | 514404 | C | T | −0.4633 |
| 1875569 | TAGGC | GAGGG | −0.357 | 354606 | C | T | −0.8001 | 514808 | T | G | −0.06641 |
| 1875589 | T | G | −0.308 | 355004 | C | A | −3.269 | 5311 | G | A | −0.7291 |
| 1875629 | A | G | −0.824 | 355860 | C | G | −3.269 | 538648 | T | G | −1.76 |
| 1875674 | A | C | −0.678 | 3569694 | T | C | −0.2811 | 540358 | ACCCA | CCCCC | −0.04754 |
| 1875771 | G | A | 1.711 | 3570229 | C | G | −2.312 | 541201 | A | G | 0.2103 |
| 1875902 | T | C | −2.881 | 3835223 | C | T | −0.8169 | 541439 | G | A | −3.251 |
| 1876531 | G | A | −0.482 | 3836358 | G | C | 1.375 | 575538 | A | G | −4.584 |
| 1876569 | G | T | −1.245 | 3986902 | G | A | −0.3308 | 575660 | CCCG | ACCC | −4.177 |
| 1876603 | C | G | −0.812 | 403565 | T | G | 0.01708 | 575688 | CGG | CG | −4.497 |
| 1877154 | G | A | −2.055 | 404130 | T | C | −0.8468 | 576439 | G | T | −3.559 |
| 1877727 | C | T | 0.078 | 405479 | G | A | −3.251 | 576699 | A | G | −0.4055 |
| 1877921 | G | T | −0.651 | 405665 | T | G | −0.7775 | 61543 | A | T | −3.313 |
| 1877947 | G | A | −0.459 | 4124351 | C | G | −3.029 | 615830 | T | G | −0.416 |
| 1878037 | T | G | −0.759 | 4228612 | T | G | −0.1715 | 616196 | A | C | −1.126 |
| 1878101 | A | G | −1.585 | 4238062 | TGGT | GGGG | −3.288 | 61898 | C | G | −1.338 |
| 1878272 | T | G | −0.128 | 4241699 | A | G | −3.667 | 61988 | C | T | −1.591 |
| 1878395 | A | G | −1.090 | 4243346 | A | G | −0.6673 | 6358 | C | T | −3.897 |
| 1878570 | T | G | −2.876 | 4243709 | C | T | −1.683 | 648774 | C | T | −2.409 |
| 1878711 | A | G | −0.372 | 4245825 | G | A | −2.73 | 6529 | G | C | −2.161 |
| 1878723 | T | G | −0.299 | 4246569 | C | T | −0.7243 | 6579 | C | T | −0.2419 |
| 1879418 | T | G | −0.353 | 4246662 | T | C | −2.297 | 6579 | C | A | −3.543 |
| 1879826 | C | G | −0.391 | 4247431 | G | A | −0.3973 | 6620 | G | A | −0.3627 |
| 1880396 | T | C | −1.741 | 4247597 | T | G | −0.2415 | 6639 | C | T | −3.925 |
| 1880402 | G | T | −1.531 | 4248320 | G | A | −4.104 | 6877 | G | T | −0.06457 |
| 1880444 | T | G | −0.854 | 4259573 | A | C | 0.02039 | 7088 | G | T | −3.135 |
| 1880629 | A | T | −0.033 | 4260790 | G | A | −0.09451 | 7151 | C | T | −3.786 |
| 1881536 | A | C | −0.987 | 4261145 | A | C | −0.3147 | 7316 | G | T | −3.462 |
| 1881713 | A | C | −0.590 | 4261150 | T | G | −0.03143 | 7495 | GCC | GCCC | −2.881 |
| 1882264 | G | C | −0.058 | 4269293 | C | T | −0.6536 | 7521 | G | T | −2.33 |
| 1883369 | T | G | −0.864 | 4269848 | T | C | −0.9397 | 7567 | A | G | −0.8127 |
| 1883385 | T | G | −0.458 | 4307601 | T | A | −0.0822 | 7581 | GACAG | TGCAC | −3.633 |
| 1884847 | G | A | −1.139 | 4308567 | G | A | −1.35 | 7582 | ACAG | CCAC | −0.3404 |
| 1885225 | G | C | −0.402 | 4326045 | CCTC | CCTCTC | −1.229 | 7582 | A | G | −0.7807 |
| 1886509 | T | G | −1.521 | 4326068 | CGG | CGGG | −5.355 | 7584 | AG | GC | −1.499 |
| 1917851 | C | A | −1.558 | 4326082 | GTC | GTTC | −3.101 | 7585 | G | C | 0.4003 |
| 1917972 | A | G | 0.698 | 4326087 | GTA | GA | −1.776 | 759857 | C | A | −3.987 |
| 1918340 | C | T | −3.252 | 4326120 | GCG | GG | −3.33 | 759917 | G | A | −4.024 |
| 1918517 | GCCC | GCCCC | −1.223 | 4326130 | CTT | CT | −2.384 | 761076 | T | G | −4.001 |
| | | C | | | | | | | | | |
| 2053589 | C | T | −0.174 | 4326181 | GAA | GAAA | −1.291 | 761100 | CAATTC | CA | −4.249 |
| | | | | | | | | | ATGGA | | |
| 2054231 | T | C | −0.065 | 4326231 | CAA | CA | −0.276 | 761128 | C | G | −3.52 |
| 2102188 | C | A | −1.708 | 4326251 | TAATTC | TAATTC | −3.112 | 761139 | C | G | −0.7744 |
| | | | | | A | AATTCA | | | | | |
| | | | | | | (SEQ ID | | | | | |
| | | | | | | NO 023) | | | | | |
| 2102289 | C | T | −0.474 | 4326270 | CAA | CA | −3.924 | 761155 | C | T | −0.2485 |
| 2102303 | G | A | −4.059 | 4326279 | ACACC | ACACCA | −1.362 | 761314 | T | C | −0.4785 |
| | | | | | | CC | | | | | |
| 2102417 | G | A | −2.459 | 4326280 | CAC | CAAC | −4.638 | 761318 | G | C | −1.192 |
| 2102778 | C | A | −4.497 | 4326292 | CTT | CT | −4.312 | 761821 | C | A | −3.876 |
| 2102849 | T | C | −0.310 | 4326301 | C | T | −4.688 | 761828 | C | G | −3.297 |
| 2102912 | G | C | −1.954 | 4326337 | G | C | −1.242 | 761973 | C | G | −1.178 |
| 2133987 | C | T | −0.151 | 4326350 | GAA | GA | −3.954 | 762399 | G | T | −3.255 |
| 2153870 | C | G | −3.494 | 4326365 | T | G | −2.6 | 762477 | A | G | −1.113 |
| 2153875 | C | G | −0.906 | 4326419 | GCC | GC | −4.787 | 763277 | A | T | −2.56 |
| 2154239 | T | C | −0.774 | 4326426 | CAAAAA | CAAAAG | −4.006 | 763552 | C | T | −0.8646 |
| | | | | | G | | | | | | |
| 2154332 | A | T | −4.052 | 4326463 | GAT | GT | −0.6911 | 764723 | T | C | −1.501 |
| 2154405 | C | A | −0.221 | 4326493 | CAG | CG | −3.18 | 764724 | T | C | 0.189 |
| 2154695 | C | G | −0.351 | 4326511 | G | C | −2.298 | 764841 | T | C | −1.581 |
| 2155168 | C | G | −0.483 | 4326547 | CTT | CT | −2.481 | 764948 | T | G | −0.2093 |
| 2223293 | T | C | −0.022 | 4326549 | T | A | −2.202 | 766198 | C | A | −1.605 |
| 2279594 | G | A | −0.436 | 4326577 | GCC | GA | −2.281 | 781687 | A | G | −0.092 |
| 2288703 | A | C | −0.973 | 4326609 | AGG | AGGG | −0.9033 | 783699 | G | A | −1.287 |
| 2288730 | G | A | −3.934 | 4326609 | AGG | AG | −1.275 | 7892 | GGCC | AGCT | −0.4027 |
| 2288782 | T | C | −2.325 | 4326613 | GTT | GT | −1.089 | 796952 | A | G | −1.197 |
| 2288892 | AGC | AGGC | −1.466 | 4326648 | ACC | AC | −3.165 | 8075 | G | T | −1.425 |
| 2289054 | T | C | −0.394 | 4326659 | AGCTGG | AGCTG | −2.126 | 8435 | C | G | −1.786 |
| | | | | | CG | GCGCT | | | | | |
| | | | | | | GGCG | | | | | |
| | | | | | | (SEQ ID | | | | | |
| | | | | | | NO 028) | | | | | |
| 2289054 | T | A | −3.789 | 4326663 | G | A | −4.024 | 8619 | T | C | −3.293 |
| 2289068 | G | T | −3.470 | 4326681 | GGAA | GGAGAA | −2.461 | 874835 | CC | CCGC | 0.07344 |
| 2289077 | ACCCG | AG | −0.967 | 4326705 | GCC | GC | −5.703 | 875005 | T | G | 0.02576 |
| 2289207 | T | G | −0.179 | 4326705 | GCCA | GA | −3.879 | 8861 | C | G | −1.346 |
| 2289223 | C | A | −3.633 | 4326707 | C | T | −2.688 | 891790 | C | T | 1.379 |
| 2518219 | T | G | −0.221 | 4326717 | AGG | AGGG | −2.412 | 892272 | G | A | −0.3085 |
| 2518864 | G | C | −0.030 | 4326718 | GGCG | GGCGC | −5.155 | 9162 | C | A | −0.107 |
| | | | | | | G | | | | | |
| 2620781 | T | C | −2.061 | 4326721 | GCT | GCCT | −4.017 | 931337 | T | C | −3.314 |
| 2620945 | G | T | −3.272 | 4326755 | C | T | −4.292 | 931398 | G | A | −0.5621 |
| 2622158 | T | G | −0.433 | 4326800 | AGCT | AGCGCT | −3.608 | 931443 | C | T | −3.251 |
| 9846 | C | G | −0.802 | | | | | | | | |

TABLE N
Para-aminosalicylic acid weighted positions; threshold value: −2.598

| POS | RN | SN | W | POS | RN | SN | W | POS | RN | SN | W |
|---|---|---|---|---|---|---|---|---|---|---|---|
| 107632 | G | T | −0.028 | 2288746 | CGG | CG | −0.02798 | 4327247 | G | T | −0.07262 |
| 1487168 | G | T | −0.034 | 2288824 | GCACAC | GC | −0.07263 | 4408009 | ACC | ACCC | −0.04536 |
| | | | | | AATGAT | | | | | | |
| | | | | | C (SEQ | | | | | | |
| | | | | | ID NO | | | | | | |
| | | | | | 029) | | | | | | |
| 1674492 | C | T | −0.032 | 2518864 | G | C | −0.0006669 | 490787 | C | G | −0.004473 |
| 1718779 | A | C | −0.016 | 2715009 | T | A | −0.02711 | 495823 | A | T | −0.03412 |
| 1851371 | ACCA | CCCC | −0.032 | 2726286 | G | A | −0.07263 | 497128 | T | G | −0.04536 |
| 1851371 | A | C | −0.073 | 2726339 | GGT | GGG | −0.04537 | 497254 | T | C | −0.07262 |
| 1875589 | T | G | −0.007 | 2832407 | A | C | −0.02353 | 511208 | T | C | −0.001959 |
| 1882264 | G | C | −0.001 | 3074258 | G | A | −0.04619 | 576500 | A | G | −0.0462 |
| 1883377 | G | C | −0.027 | 348383 | A | G | −0.0462 | 615805 | T | G | −0.009441 |
| 1883761 | T | G | −0.024 | 3569297 | A | C | −0.01275 | 615830 | T | G | −0.01627 |
| 1884966 | A | G | −2.058 | 4124895 | A | C | −0.01907 | 761140 | A | G | −0.07265 |
| 2053589 | C | T | −0.001 | 4242643 | C | T | 0.001231 | 764822 | G | T | −0.04536 |
| 2102912 | G | C | −0.073 | 4243331 | G | T | −0.03403 | 765842 | A | G | −0.02639 |
| 2154405 | C | A | −0.003 | 4249293 | A | G | −0.01863 | 765882 | C | G | −0.07262 |
| 2279349 | T | G | −0.010 | 4260093 | A | G | −0.02642 | 797168 | T | G | −0.0462 |
| 9162 | C | A | −0.001 | | | | | | | | |
| 9382 | G | A | −0.034 | | | | | | | | |

TABLE O
Cycloserine weighted positions; threshold value: −1.859

| POS | RN | SN | W | POS | RN | SN | W |
|---|---|---|---|---|---|---|---|
| 1417272 | T | G | −1.083 | 404130 | T | C | −0.004 |
| 1879405 | G | T | −0.406 | 4239274 | C | G | −0.401 |
| 1883750 | T | G | −0.584 | 4240664 | T | G | −1.191 |
| 1918034 | T | G | −0.451 | 4243346 | A | G | −0.030 |
| 2714913 | C | T | −0.399 | 4308785 | T | G | −0.022 |
| 355709 | A | C | −0.002 | 4399683 | C | G | −0.003 |

TABLE P
Rifabutin weighted positions; threshold value: −0.605

| POS | RN | SN | W |
|---|---|---|---|
| 2937852 | A | C | −0.067 |
| 4238859 | G | T | −0.283 |

TABLE Q
Levofloxacin weighted positions; threshold value −2.358

| POS | RN | SN | W | POS | RN | SN | W | POS | RN | SN | W |
|---|---|---|---|---|---|---|---|---|---|---|---|
| 1759252 | G | T | −0.908 | 1883385 | T | G | −0.2856 | 4247249 | G | C | −1.37 |
| 1850010 | C | T | −0.071 | 1883750 | T | G | −1.249 | 4247395 | C | T | −0.3379 |
| 1851349 | A | C | −0.332 | 1884791 | T | G | −0.1154 | 4249401 | T | G | −0.5473 |
| 1876332 | GGGC | CGGG | −1.384 | 2279407 | G | A | −0.2353 | 4256011 | G | A | −1.079 |
| 1877652 | G | A | −0.340 | 2288739 | G | A | −0.05764 | 4399629 | C | G | −0.2104 |
| 1878037 | T | G | −0.058 | 2289082 | G | A | −0.5474 | 495491 | T | C | −0.5473 |
| 1882668 | T | C | −0.859 | 2693848 | G | T | −0.04647 | 538621 | C | G | −0.2349 |
| 1883369 | T | G | −0.031 | 346199 | G | A | −0.9649 | 760314 | G | T | −0.05279 |
| 7862 | C | T | −0.547 | | | | | | | | |
| 801174 | T | C | −0.338 | | | | | | | | |

Tables A-Q show reference and sample positions and weights for a set of 17 antibiotics. To assess resistance or susceptibility to any of the antibacterial drugs listed, one has to sum the weights in column W for every position in column POS where the sample nucleotide SN is found in the sample instead of reference nucleotide RN and then compare this sum to the threshold provided for this antibiotic in Table 2. If the sum of weights is below or equal to the threshold it is considered resistant, if it is larger than the threshold, the sample is predicted to be susceptible

TABLE 2
Comparison of predicted phenotype to measured phenotype on DNA from clinical isolates.

| | | | | | | | | | Para- | | | | | | | | | |
|---|---|---|---|---|---|---|---|---|---|---|---|---|---|---|---|---|---|---|
| | | | | | | | | | aminosalysic | | | | | | | | | |
| | Isoniazid | | Rifampicin | | Amikacin | | Moxifloxacin | | acid | | Cycloserine | | Capreomycin | | Pyrazinamide | | Kanamycin | |
| Isolate | F | P | F | P | F | P | F | P | F | P | F | P | F | P | F | P | F | P |
| DNA_1 | R | R | R | R | S | S | S | S | R | S | S | S | S | S | S | S | S | S |
| DNA_2 | R | R | R | R | S | S | S | S | S | S | S | S | S | S | R | S | S | S |
| DNA_3 | R | R | R | R | S | S | S | S | S | S | S | S | S | S | R | R | S | S |
| DNA_4 | R | R | R | R | S | S | S | S | S | S | S | S | R | S | R | R | S | S |
| DNA_5 | R | R | R | R | S | S | S | S | S | S | S | S | S | S | R | R | S | S |
| DNA_6 | R | R | R | R | S | S | S | S | R | R | S | S | S | S | R | R | R | S |
| DNA_7 | R | R | R | S | S | S | S | S | S | S | S | S | S | S | R | S | S | S |
| DNA_8 | R | R | R | R | S | S | S | S | S | S | S | S | S | S | R | R | S | S |
| DNA_9 | R | R | R | R | S | S | S | S | S | S | S | S | S | S | R | S | S | S |
| DNA 10 | R | R | R | R | S | S | S | S | S | S | S | S | S | S | R | R | S | S |
| DNA 11 | R | R | R | R | S | S | S | S | S | S | S | S | S | S | S | S | — | R |
| DNA 12 | R | R | R | R | S | S | S | S | S | S | S | S | S | S | R | R | — | S |

Sequencing data was obtained via targeted sequencing, covering all positions in tables A-Q. F: Phenotype; P: Prediction; R: resistant, S: susceptible, I: intermediary. —: missing measurement

1. A method for predicting mycobacterial resistance to an antibacterial drug in a patient, comprising the following steps:
a. isolating mycobacterial nucleic acid from a sample obtained from said patient;
b. a sequencing step, wherein a nucleic acid sample sequence from said mycobacterial nucleic acid is obtained;
c. an alignment step, wherein said sample sequence is aligned to a reference sequence, wherein said reference sequence is the sequence NC_000962.3, and wherein the reference sequence comprises a plurality of reference positions,
wherein said sequencing step is designed to obtain a sample sequence capable of aligning to all of the positions identified in a table selected from Tables A, B, C, D, E, F, G, H, I, J, K, L, M, N, O, P and/or Q;
d. a comparison step, wherein said sample sequence is compared to said reference sequence in at least 90% of said plurality of reference positions identified in said table, and wherein for each reference position, it is determined whether the sample sequence value S^{R} is the same as the sample sequence value S^{N} assigned to said position in said table;
if S^{R} is the same as S^{N}, assigning the value associated to S^{N} in the table as the position weight value to the position;
if S^{R} is not the same as S^{N}, assigning the value 0 as the position weight value to the position;
thereby obtaining a position weight value assigned to each of said reference positions, each of said position weight values being associated to an antibacterial drug designated in said table;
e. obtaining a predictor value to predict resistance against said antibacterial drug, by adding all position weight values to obtain the predictor value;
f. comparing the predictor value to the threshold value identified in the header of said table, wherein
if the predictor value is equal or smaller than the threshold value, resistance to the antibacterial drug is predicted and
if the predictor value is larger than the threshold value, susceptibility to the antibacterial drug is predicted.

2. The method for predicting mycobacterial resistance to an antibacterial drug in a patient according to claim 1, wherein the comparison step comprises comparing said sample sequence to said reference sequence in each of said plurality of reference positions.

3. The method according to claim 1, wherein resistance or susceptibility to an antibacterial drug is determined for more than one drug by comparing the sample sequence to more than one of Tables A to Q.

4. The method according to claim 2, wherein drug resistance or susceptibility is determined for six antibacterial drugs, particularly for eight antibacterial drugs, more particularly for ten or twelve, or even for fourteen or sixteen antibacterial drugs.

5. The method according to claim 3, wherein drug resistance or susceptibility is determined for a plurality of antibacterial drugs including at least one, particularly two, three or even all of ciprofloxacin, para-aminosalycylic acid, cycloserine and rifabutin.

6. The method according to claim 1, wherein drug resistance is determined for Isoniazid, Rifampicin, Ethambutol, Pyrazinamide, Streptomycin, Ciprofloxacin, Moxifloxacin, Ofloxacin, Amikacin, Capreomycin, Kanamycin, Prothionamide, Ethionamide, Paraaminosalicylic acid, Cycloserine, Rifabutin and Levofloxacin.

7. A system comprising a sequencing means and a computer programmed to carry out the method of claim 1.

8. An antibacterial drug selected from Isoniazid, Rifampicin, Ethambutol, Pyrazinamide, Streptomycin, Ciprofloxacin, Moxifloxacin, Ofloxacin, Amikacin, Capreomycin, Kanamycin, Prothionamide, Ethionamide, Paraaminosalicylic acid, Cycloserine, Rifabutin and Levofloxacin for use in treatment of a patient suffering from infection by a mycobacterial strain, wherein susceptibility of said mycobacterial strain is determined by a method according to claim 1.

9. An antibacterial drug for use in treatment of a patient suffering from infection by a mycobacterial strain, wherein said mycobacterial strain has been determined to be susceptible to said drug by a method according to claim 1.


SEQUENCE LISTING (30 sequences)

SEQ ID NO: 1 - DNA, 11 bp, Mycobacterium tuberculosis
caattcatgga

SEQ ID NO: 2 - DNA, 14 bp, Mycobacterium tuberculosis
cgacgaggtcgtgg

SEQ ID NO: 3 - DNA, 11 bp, Mycobacterium tuberculosis
ccaccgccagc

SEQ ID NO: 4 - DNA, 10 bp, Mycobacterium tuberculosis
tgacccacaa

SEQ ID NO: 5 - DNA, 12 bp, Mycobacterium tuberculosis
ggccgtctggcg

SEQ ID NO: 6 - DNA, 22 bp, Mycobacterium tuberculosis
ggccgtctggcgccgtctggcg

SEQ ID NO: 7 - DNA, 72 bp, Mycobacterium tuberculosis
acgcaaaagctcccaaatccgaccggatttgggagcttttgcgtcttttcgcggtggtcagccgcggcggcc

SEQ ID NO: 8 - DNA, 19 bp, Mycobacterium tuberculosis
ggtctcttcggagatactc

SEQ ID NO: 9 - DNA, 36 bp, Mycobacterium tuberculosis
ggtctcttcggagatactgtctcttcggagatactc

SEQ ID NO: 10 - DNA, 10 bp, Mycobacterium tuberculosis
ggtttggttc

SEQ ID NO: 11 - DNA, 12 bp, Mycobacterium tuberculosis
ggctggtcgttg

SEQ ID NO: 12 - DNA, 14 bp, Mycobacterium tuberculosis
ggcgttcgcgttcg

SEQ ID NO: 13 - DNA, 12 bp, Mycobacterium tuberculosis
atctcctctcct

SEQ ID NO: 14 - DNA, 13 bp, Mycobacterium tuberculosis
gcgcggcgccacc

SEQ ID NO: 15 - DNA, 29 bp, Mycobacterium tuberculosis
ggcgccaccggttaccgccagcgagccac

SEQ ID NO: 16 - DNA, 13 bp, Mycobacterium tuberculosis
gccagcgagccac

SEQ ID NO: 17 - DNA, 11 bp, Mycobacterium tuberculosis
tgcgtaccgcg

SEQ ID NO: 18 - DNA, 20 bp, Mycobacterium tuberculosis
tgcgtaccgcgcgtaccgcg

SEQ ID NO: 19 - DNA, 11 bp, Mycobacterium tuberculosis
ccgaccacatc

SEQ ID NO: 20 - DNA, 17 bp, Mycobacterium tuberculosis
cacatcgacctcatcga

SEQ ID NO: 21 - DNA, 11 bp, Mycobacterium tuberculosis
cgtgctcgtgc

SEQ ID NO: 22 - DNA, 20 bp, Mycobacterium tuberculosis
cgtgctcgtggtgctcgtgc

SEQ ID NO: 23 - DNA, 12 bp, Mycobacterium tuberculosis
taattcaattca

SEQ ID NO: 24 - DNA, 14 bp, Mycobacterium tuberculosis
gggggcgggaggcg

SEQ ID NO: 25 - DNA, 13 bp, Mycobacterium tuberculosis
gcacacaatgatc

SEQ ID NO: 26 - DNA, 24 bp, Mycobacterium tuberculosis
cgacggactggtctggcttcaccg

SEQ ID NO: 27 - DNA, 12 bp, Mycobacterium tuberculosis
gattccattcca

SEQ ID NO: 28 - DNA, 14 bp, Mycobacterium tuberculosis
agctggcgctggcg

SEQ ID NO: 29 - DNA, 13 bp, Mycobacterium tuberculosis
gcacacaatgatc

SEQ ID NO: 30 - DNA, 12 bp, Mycobacterium tuberculosis
ccccccgcccca